SEQUENCE LISTING (64 sequences)

SEQ ID NO: 1 - DNA, 1683 bp, Influenza A Virus
  source 1..1683: mol_type = unassigned DNA
atgtacaaaatagtactagtacttgcgctccttggagcggtgcatggtcttgacaaaatatgccttggacatcatgcagtccccaatggcaccatcgtaaagactctcacaaacgaaaaggaagaggtgaccaatgctactgaaacggtggaaagtaaaagcctggacaaactttgcatgaaaagtcggaattacaaggacctaggtaattgccacccgatagggatggtgatagggactcctgcttgtgacttacacctcaccggaacatgggacactttgatagagagagacaattccattgcctactgttacccaggtgccactgtgaatgaagaagcattaaggcagaaaattatggaaagtggagagattgacaagataagcaccgggtttacatatgaatcatccatcaatccagctggaaccactaaagcatgcatgagaaatgggaaaaacagtttctatgcagagctaaagtggctagtgtcgaaggacaaaggacggaacttcccacaaacaacaaacacatacaggaatacagattcaacagaacaccttataatctggggaattcatcacccgtcaagcacacaagaaaagaatgatctgtatggaacacaatcactttccatttcagtagggagttctacttatcaaaacaactttgtgcctgtggtgggagcaagaccacaggtgaatggccaaagtgggcggattgatttccattgggcgatggtacaaccgggtgataacatcactttttcgcataacggcggactaatagcacctagtagagtgagtaaactaaagggaagaggccttggcattcaatcaggagcttcagtagataatgactgtgaatcaaaatgtttttggaaaggtggatccatcaacaccaaactcccttttcagaatctttccccaagaactgtgggtcaatgccccaagtatgtgaacaaaaagagcctgttgcttgctaccggaatgaggaatgtgccagaggttgtccaaggaagaggcctgttcggagcaattgctggattcatagaaaatggatgggaagggatggtagatggttggtatggtttccgacatcaaaatgcccaaggcactggtcaggctgcggattacaaaagcactcaggcagctatagatcaaatcaccgggaaattgaacagactgatagagaagacaaacacagagttcgaatccatagaatctgagttcagtgaaattgaacatcaaattggcaatgtaataaactggactaaggattcgataacagacatttggacgtatcaagctgaattactggtagcaatggaaaaccagcatacaatcgacatggctgattcagaaatgctgaatctatatgagagagtgaggaagcaactgaggcaaaatgcagaagaagatgggaaagggtgctttgaaatatatcacaaatgcgacgacaactgcatggaaagcatcagaaacaacacctatgaccatacacaatacagagaagaagcactcttgaacagactcaacattaatccggtgaaactctcttctgggtacaaagatgttatactgtggtttagcttcggggcgtcatgctttgtacttttggctgtcatcatggggcttgttttcttctgtctgaaaaatggaaacatgcgatgcacaatctgtatt

SEQ ID NO: 2 - DNA, 1683 bp, unidentified
  misc_feature 1..1683: note = deoptimized Influenza A Virus
  source 1..1683: mol_type = other DNA
atgtataagatagtgctcgtactcgcactattaggcgcagtgcacggactcgacaaaatttgcctagggcatcacgcagtgcctaacggaactatcgttaagacacttactaacgaaaaagaggaagtgactaacgctaccgaaacagtcgaatcaaaatcactcgacaaattgtgtatgaaaagtcggaattataaagacctaggcaattgccatccgatagggatggtgatagggactcccgcttgcgatctgcatctgacagggacatgggatacacttatcgaacgggacaatagtatagcgtattgttatccaggcgctacagtgaacgaagaggcacttagacaaaaaattatggaatccggcgaaatcgataagattagtaccggattcacatacgaatcctctattaatcccgcaggaacaactaaggcttgtatgcgaaacggtaagaattcgttttacgctgaactgaaatggcttgtgagtaaggacaaaggtaggaatttcccacaaactactaatacttataggaataccgattcaaccgaacatctgattatatgggggatacaccatccaagttcgacacaagagaaaaacgatctatacggaacgcaatcccttagcattagcgtagggtctagtacttatcagaataatttcgtaccggtagtgggcgctagaccgcaagtgaacggacaatccggtagaatcgatttccattgggctatggtgcaaccaggcgataacataacttttagccataacggcggactgatagcgcctagtagagtgagtaagcttaagggaagggggttggggatacaatccggcgctagcgtagacaacgattgcgaatcaaaatgcttttggaaaggggggtcaattaatactaaattgccatttcagaatctgtcacctagaacagtgggacaatgccctaaatacgttaataagaaaagtctgttactcgcaaccggtatgcgaaacgtaccagaggtagtgcaaggtagggggctattcggagcgatagcgggatttatcgaaaacggatgggagggtatggtcgacggatggtacgggtttagacaccaaaacgcacagggaaccggacaggcagcagactataaatcgacacaagccgctatagaccaaattaccggtaagcttaacagactgatcgaaaagactaataccgaattcgaatcaatcgaatccgaatttagcgaaatcgaacaccaaatcggaaacgtaattaattggacaaaagactcaattaccgatatatggacatatcaagccgaactgttagtcgctatggagaatcagcatacaatcgatatggccgatagcgaaatgcttaacctttacgaaagggtgagaaaacagcttagacaaaacgctgaagaggacggtaaggggtgtttcgaaatataccataaatgcgacgataattgtatggagtctatacggaataacacatacgaccatacgcaatatagagaggaagcactactgaatagacttaacattaatccggttaagctatctagcggatataaagacgtgatattgtggttctcattcggagcgtcatgtttcgtattgctcgcagtgattatgggactcgtattcttttgccttaaaaacggtaatatgagatgcacaatttgcata

SEQ ID NO: 3 - DNA, 1410 bp, Influenza A virus
  source 1..1410: mol_type = unassigned DNA
atgaatcctaatcaaaaattattcgcactctctggggtggccatagcactgagtatcctcaacctactaataggaatatccaatgtgggactgaatgtctcactacacctgaagggaagcagtgaccaggataagaattggacatgcacgagtgtaacacaaaccaacacgactttaatcgaaaacacgtatgtcaacaataccactgtcatcaataaggaaacagggactacaaagcaaaattatctaatgctgaacaagagtttatgcaaagttgaaggatgggtagtggtggccaaggacaatgccataagattcggtgaaagtgaacaaataatagtgacaagggagccgtatgtgtcatgtgatccattaggatgtaagacgtacgcactgcatcaagggacaaccattagaaacaagcactcaaacggaacaatacacgacaggactgctttcagagggttgatatcaactcctttggggagcccccctgtagtcagcaatagtgactttctttgtgtagggtggtcaagcaccagttgccatgacggcatcgggcggatgaccatttgcgtgcagggaaataataacaacgcaacagctacagtgtactatgaccgaaggctcactaccacaataaaaacatgggcagggaaaatccttaggacgcaagagtcggaatgtgtatgccacaatggaacatgtgtagtaataatgaccgatggatcggcaagcagccaggcacatacaaaagttctgtatttccacaaaggactagtaataaaagaggaagccctcaagggatcagccagacacatagaggagtgctcatgctatgggcacaattcaaaggtgacttgtgtatgcagggacaactggcaaggagccaatagaccagtgattgaaatagatatgaatgccatggagcatacaagtcagtatctatgtacaggagttctcactgacacgagcagaccatcagacaaatcaatgggcgactgtaataatccgatcactgggagtccgggagcccctggggtcaaaggattcggcttcctggatagtgacaatacatggttgggccgcacaataagtcctcgttccaggagtggttttgagatgttgaagatacctaatgctgggacagacccaaattctagaatcactgagaggcaagaaatagttgacaacaacaattggtcaggatactcaggaagtttcattgactattgggatgaaagcagtgtgtgctacaacccctgtttttatgttgaattaataagaggaaggcctgaagaagccaagtatgtttggtggacgagcaacagtttagttgcactatgtggaagcccaatctcagttgggtccggttccttccccgatggggcacaaatccaatacttttcg

SEQ ID NO: 4 - DNA, 1410 bp, unidentified
  misc_feature 1..1410: note = Deoptimized Influenza A virus
  source 1..1410: mol_type = other DNA
atgaatcctaaccaaaagctattcgcactaagcggagtcgccatagccctatcaatactgaatctgttaatcggaatatcgaacgttgggttgaacgttagtttgcaccttaaggggtcatccgaccaagacaaaaattggacatgtactagcgttacgcaaacaaatacgactttgatcgaaaatacatacgttaacaatacgacagtgataaataaagagaccggaactactaagcaaaactatctgatgctgaataagtcactatgtaaggtcgagggatgggtggtagtcgctaaagacaacgcaataaggttcggcgaaagcgaacagataatcgtgacacgcgaaccatacgttagttgcgatccgttagggtgtaagacatacgcattacaccaagggactacgatacggaataaacactctaacggaacgatacacgacagaaccgcatttagggggttgatatcgacacctctcggatcacctcccgtagtgagtaatagcgatttcttatgcgtggggtggtcaagtactagttgtcacgacggaatcggacgtatgacaatatgcgtacaggggaataacaataacgcaaccgcaacagtgtattacgataggagactgactacaacaattaagacttgggccggtaagatactgagaacacaggaaagcgaatgcgtttgccataacggtacatgcgtagtgattatgacagacggatccgcaagttcgcaagcccatacgaaagtgctatattttcacaaagggctcgtaatcaaagaggaagcccttaagggatccgctagacatatcgaagagtgtagttgttacggacacaatagtaaggttacatgcgtatgtagggacaattggcaaggcgcaaatagaccagtgatagagatagacatgaacgctatggagcatacgagtcagtatctatgtaccggagtgttaaccgacactagtagacctagcgataagagtatgggcgattgcaataatccgataaccggatcacccggagcaccaggcgttaaggggttcgggtttctcgatagcgataatacatggttaggtaggacaatctcacctaggtcaagatccggattcgaaatgctcaaaatccctaacgccggaacagaccctaatagtaggattaccgaacgacaagagatagtcgacaataacaattggtcagggtatagcggatctttcatagactattgggacgaatcaagcgtatgttataacccatgtttctatgtcgaactgattagggggagacccgaagaggccaaatatgtgtggtggactagtaatagtctcgtagccctatgcggatcaccgataagcgtagggtcagggtcattcccagacggagcccaaatccaatattttagt

SEQ ID NO: 5 - DNA, 1698 bp, Influenza A virus
  source 1..1698: mol_type = unassigned DNA
atgaaggcaatactagtagttctgctatatacatttgcaaccgcaaatgcagacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggacatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctgggtctggtattatcatttcagatacaccagtccacgattgcaatacaacttgtcagacacccaagggtgctataaacaccagcctcccatttcagaatatacatccgatcacaattggaaaatgtccaaaatatgtaaaaagcacaaaattgagactggccacaggattgaggaatgtcccgtctattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatt

SEQ ID NO: 6 - DNA, 1698 bp, unidentified
  misc_feature 1..1698: note = Deoptimized Influenza A virus
  source 1..1698: mol_type = other DNA
atgaaagcgattctagtcgtactgctatatacattcgctaccgctaacgccgatacactatgcatagggtatcacgctaataatagtacagacacagtagacacagtactcgaaaaaaacgttacggttacacattccgttaatctgttagaggataagcataacggtaagctatgtaaactgagaggcgtagcaccattgcatttgggtaagtgtaatatagccggatggatactaggtaatcccgaatgcgaatcactatcaactgcaagttcatggtcttatatagtcgaaactagttcaagcgataacggtacatgttatcccggagactttatcgattacgaagagttgagagagcaattgtctagcgtaagctcattcgaaagattcgaaatttttccgaaaactagttcatggcctaatcacgattcaaataagggggtaacagccgcatgcccacacgcaggcgctaagtcattctataaaaatctgatatggctagtgaaaaaagggaattcttatccgaaactatcaaaatcatatattaacgataagggtaaggaggtactcgtattgtgggggatacaccatccatcaactagcgcagaccaacaatctctgtatcagaatgccgacgcatacgtattcgtagggactagtaggtactctaaaaaatttaaacccgaaatcgctattagaccgaaagtgagagaccaggagggaagaatgaattactattggacactagtcgaaccaggcgataagattacattcgaagcgacagggaatctagtggtaccgagatacgcattcgcaatggagagaaacgccggatccggaattattattagcgatactcccgtacacgattgcaatacaacatgtcagacaccaaaaggggcaattaatactagcctaccatttcagaatatacacccaattacaatcggtaagtgtccaaaatacgttaagtctacgaaacttagattggcaacagggttgagaaacgtaccatcaatacagtctagagggttgttcggagcaatcgccggattcatagagggggggtggaccggtatggtcgacggatggtacggataccatcatcaaaacgaacaggggtccggatacgcagccgatctgaaatcaacacagaacgcaatcgacgaaattacgaataaagtgaatagcgtaatcgaaaaaatgaatactcagtttacagccgtaggtaaggaatttaatcatctcgaaaaaagaattgagaatctgaataaaaaggtagacgacgggtttctagacatttggacatataatgccgaactgttagtgttactcgaaaacgaaagaacattagactatcacgattctaacgttaagaatctatacgaaaaagtgagatcgcaattgaagaataacgcaaaagagatagggaatgggtgtttcgaattctaccataaatgcgataatacatgtatggaatccgtaaaaaacggtacatacgattatccgaaatatagcgaagaagcaaaactgaatagggaagagattgacggagttaagttggagtcaactaggatttaccagatactcgcaatttactctacagtcgcatcaagtctagtgttagtcgttagcttaggcgcaattagtttttggatgtgttcaaacggatcactgcaatgtaggatttgcata

SEQ ID NO: 7 - DNA, 1407 bp, Influenza A virus
  source 1..1407: mol_type = unassigned DNA
atgaatccaaaccaaaagataataaccattggttcggtctgtatgacaattggaatggctaacttaatattacaaattggaaacataatctcaatatggattagccactcaattcaacttgggaatcaaaatcagattgaaacatgcaatcaaagcgtcattacttatgaaaacaacacttgggtaaatcagacatatgttaacatcagcaacaccaactttgctgctggacagtcagtggtttccgtgaaattagcgggcaattcctctctctgccctgttagtggatgggctatatacagtaaagacaacagtataagaatcggttccaagggggatgtgtttgtcataagggaaccattcatatcatgctcccccttggaatgcagaaccttcttcttgactcaaggggccttgctaaatgacaaacattccaatggaaccattaaagacaggagcccatatcgaaccctaatgagctgtcctattggtgaagttccctctccatacaactcaagatttgagtcagtcgcttggtcagcaagtgcttgtcatgatggcatcaattggctaacaattggaatttctggcccagacaatggggcagtggctgtgttaaagtacaacggcataataacagacactatcaagagttggagaaacaatatattgagaacacaagagtctgaatgtgcatgtgtaaatggttcttgctttactgtaatgaccgatggaccaagtgatggacaggcctcatacaagatcttcagaatagaaaagggaaagatagtcaaatcagtcgaaatgaatgcccctaattatcactatgaggaatgctcctgttatcctgattctagtgaaatcacatgtgtgtgcagggataactggcatggctcgaatcgaccgtgggtgtctttcaaccagaatctggaatatcagataggatacatatgcagtgggattttcggagacaatccacgccctaatgataagacaggcagttgtggtccagtatcgtctaatggagcaaatggagtaaaaggattttcattcaaatacggcaatggtgtttggatagggagaactaaaagcattagttcaagaaacggttttgagatgatttgggatccgaacggatggactgggacagacaataacttctcaataaagcaagatatcgtaggaataaatgagtggtcaggatatagcgggagttttgttcagcatccagaactaacagggctggattgtataagaccttgcttctgggttgaactaatcagagggcgacccaaagagaacacaatctggactagcgggagcagcatatccttttgtggtgtaaacagtgacactgtgggttggtcttggccagacggtgctgagttgccatttaccattgacaag

SEQ ID NO: 8 - DNA, 1407 bp, unidentified
  misc_feature 1..1407: note = Deoptimized Influenza A virus
  source 1..1407: mol_type = other DNA
atgaatcctaaccaaaaaattataacaatcggatccgtttgtatgacaatcggtatggctaacctaatactgcaaatcggtaatattatatcgatttggatctcacatagtatacaattgggtaatcagaatcagatagagacatgcaatcaatccgttattacatacgaaaataatacttgggttaatcagacatacgttaacatatcgaatactaatttcgctgccggacaatccgtcgttagcgttaagttagccggtaatagttcactatgccccgttagcgggtgggctatatactctaaagacaattcgattagaatcggatctaagggcgacgtattcgtaatacgcgaaccattcataagttgtagtccattagagtgtagaactttttttctaacacaaggcgctctattgaacgataagcatagtaacggtacaattaaggatagatcaccttatagaacattgatgtcatgtcctatcggcgaagtgcctagtccatacaatagtagattcgaatccgtcgcatggtccgctagcgcatgtcacgacgggattaattggttgactatagggattagcggacccgataacggcgcagtcgctgtgcttaagtataacggtattattaccgacactataaagagttggcgaaataacatactgagaacacaggaatccgaatgcgcatgcgtaaacggttcatgttttaccgtaatgactgacggacctagcgacggacaagcgtcatataagatttttagaatcgaaaaaggtaagatagtgaaatctgtcgagatgaacgctccgaattatcattacgaagagtgtagttgttatcccgattctagcgaaattacatgcgtatgtagggacaattggcacgggtctaatcgaccatgggtgtcattcaatcagaacttagagtatcagatagggtatatatgctcagggatattcggcgataatcctagaccgaacgataaaaccggatcatgcggaccagtgtcatctaacggcgctaacggagtgaaagggtttagtttcaaatacggtaacggcgtatggatcggacgaactaagtctatatctagtaggaacggattcgaaatgatatgggacccaaacgggtggaccggtaccgataataacttttcaatcaaacaggacatagtcggaattaacgaatggtccgggtatagcggatcattcgtgcaacatccagagttaaccggactcgattgcataagaccatgtttttgggtcgaattgattagggggagaccaaaagagaatactatatggactagcggatctagtattagcttttgcggagtgaatagcgataccgtagggtggtcatggccagacggagccgaactaccatttacaatcgataag

SEQ ID NO: 9 - DNA, 1698 bp, Influenza A virus
  source 1..1698: mol_type = unassigned DNA
atgaaagtaaaactactgatcctgttatgtacatttacagctacatatgcagacacaatatgtataggctaccatgccaacaactcaaccgacactgttgacacagtacttgagaagaatgtgacagtgacacactctgtcaacctacttgaggacagtcacaatggaaaactgtgcctactaaaaggaatagcccccctacaattgggtaattgcagcgttgccggatggatcttaggaaacccagaatgcgaattactgatttccaaggaatcatggtcctacattgtagaaacaccaaatcctgagaatggagcatgttacccagggtatttcgccgactatgaggagctaagggagcaattgagttcagtatcttcatttgagagattcgaaatattccccaaagaaagctcatggcccaaccacaccgtaaccggagtatcagcatcatgctcccataatgggaaaagcagtttttacaaaaatttgctatggctgacggggaagaatggtttgtacccaaacctgagcaagtcctatgcaaacaacaaagagaaagaagtccttatactatggggtgttcatcacccgcctaacataggggaccaaaggaccctctatcacacagaaaatgcttatgtctctgtagtgtcttcacattatagcagaagattcaccccagaaataaccaaaaggcccaaagtaagagatcaggaaggaagaatcaactactactggactctgctggaacccggggatacaataatatttgaggcaaatggaaatctaatagcgccatggtatgctttcgcactgagtagaggctttggatcaggaatcatcacctcaaatgcaccaatggatgaatgtgatgctaagtgtcaaacacctcagggagctataaacagcagtcttcctttccagaatgtacacccagtcacaataggagagtgtccaaagtatgtcaggagtgcaaaattaaggatggttacaggactaaggaacatcccatccattcaatccagaggtttgtttggagccattgccggtttcattgaaggggggtggactggaatggtagatgggtggtatggttatcatcatcagaatgagcaaggatctggctatgctgcagatcaaaaaagcacacaaaatgccattaacgggattacaaacaaggtgaattctgtaattgagaaaatgaacactcaattcacagctgtgggcaaagaattcaacaaattggaaagaaggatggaaaacttaaataaaaaggttgatgatgggtttctagacatttggacatataatgcagaattgttggttctactggaaaatgaaaggactttggatttccacgactccaatgtgaagaatctgtacgagaaagtaaaaagccaattaaagaataatgccaaagaaataggaaatgggtgttttgaattctatcacaagtgtaacaatgaatgcatggagagtgtgaaaaatggaacttatgactatccaaaatattccgaagaatcaaagttaaacagggaaaaaattgatggagtgaaattggactcaatgggggtctatcagattctggcgatctactcaactgtcgccagttccctggttcttttggtctccctgggggcaatcagcttctggatgtgttccaatgggtctttgcagtgtagaatatgcatctga

SEQ ID NO: 10 - DNA, 1698 bp, unidentified
  misc_feature 1..1698: note = Deoptimized Influenza A virus
  source 1..1698: mol_type = other DNA
atgaaagtgaaactgttaatactgttgtgcacttttaccgctacatacgccgatacaatttgcatagggtatcacgctaataatagtaccgatacagtcgacactgtgttggaaaagaacgtaaccgttacacactccgttaatctgttagaggattcccataacggtaagttgtgtctgttgaaagggatcgcaccattgcaattgggtaattgtagcgtagccggatggatattggggaatcccgaatgcgaactattgattagtaaagagtcatggtcatatatagtcgagacacctaatcccgaaaacggagcatgctatcccggatatttcgccgattacgaagagcttagagagcaattgtctagcgtaagctcattcgaaagattcgaaatttttccaaaagagtcaagttggcctaatcataccgtaacaggcgtatccgcatcatgtagtcataacggtaagtcaagcttttataagaatctgttatggttaaccggtaaaaacggactgtatccaaatctatctaagtcatacgcaaataataaagagaaagaggtactgattctatggggggtgcatcacccacctaatataggcgatcaaagaacattgtatcataccgaaaacgcatacgtatccgtcgttagctcacactatagtagaaggtttacacccgaaattactaagagacctaaggtaagggatcaggagggtaggattaattattattggactctacttgaaccaggcgatactatcatattcgaagctaacggaaatctaatcgcaccatggtacgcattcgcactatctagggggttcggatccgggattattacttctaacgctccaatggacgaatgcgacgcaaagtgtcagacaccacagggagcgattaatagttccctaccattccaaaacgtacaccccgttacaatcggcgaatgtccgaaatacgttagatccgctaaacttagaatggtgaccggactgagaaatataccatcaatccaatctagggggctattcggagccatagccggatttatcgaaggggggtggacagggatggtcgacggatggtatgggtatcaccaccaaaacgaacagggatccggatacgccgccgatcagaaatccacacaaaacgctattaacggaattacgaataaagtgaatagcgtaatcgaaaaaatgaatacacaatttactgccgtaggtaaggaattcaataagttagagagaaggatggagaatctgaataaaaaagtcgacgacggattcctagacatatggacatataacgccgaactgttagtgttgcttgagaacgaaaggacactagactttcacgattcaaacgttaaaaatctatacgaaaaagtcaaatcccaattgaaaaataacgctaaagagatagggaatgggtgtttcgaattctatcataagtgtaataacgaatgtatggaatccgttaaaaacggaacatacgattatccaaagtatagcgaagagtcaaaactgaatagggaaaaaatcgacggagtcaaacttgactcaatgggggtgtatcagatactcgcaatctatagtacagtcgcatctagcctagtactgttagtgagtctgggagcgataagcttttggatgtgttctaacggatcactgcaatgtaggatatgcatatga

SEQ ID NO: 11 - DNA, 1410 bp, Influenza A virus
  source 1..1410: mol_type = unassigned DNA
atgaatccaaatcaaaaaataataacgattggctctgtttctctcaccattgccacaatatgcttccttacgcaaattgccatcctggtaactactgtaacattgcatttcaagcaatatgaatgcaactcccccccaaacaaccaagtgatgctgtgtgaaccaacaataatagaaagaaacataacagagatagtgtatctgaccaacaccaccatagagaaggaaatatgccccaaactagcagaatacagaaattggtcaaagccgcaatgcaacattactggatttgcacctttttctaaggacaattcgattcggctttccgctggtggggacatctgggttacaagagaaccttatgtgtcatgcgatcctgacaagtgttatcaatttgcccttggacagggaacaacactaaacaacgggcattcaaatgacacagtacatgataggaccccttataggaccctattgatgaatgagttgggtgttccatttcatttgggaaccaagcaagtgtgcatagcatggtccagctcaagttgtcacgatggaaaagcatggctgcatgtttgtgtaacgggggatgataaaaatgcaactgctagcttcatttacaatgggaggcttgtagatagtataggttcatggtccaaaaaaatcctcaggacccaggagtcggaatgcgtttgtatcaatggaacttgtacagtagtaatgactgatgggagtgcttcaggaaaagctgatactaaaatactattcattgaggaggggaaaatcgttcatactagcctattgtcagggagtgctcagcatgtcgaggagtgctcctgttatcctcgatatcctggtgtcagatgtgtctgcagagacaactggaaaggctccaataggcccatcgtagatataaatgtaaaggattatagcattgtttccagttatgtgtgctcaggacttgttggagacacacccagaaaaaacgacagctccagcagtagccattgcttggatcctaacaatgaggaaggtggtcatggagtgaaaggctgggcctttgatgatggaaatgacgtgtggatgggaagaacgatcagcgagaagttacgctcaggatatgaaaccttcaaagtcattgaaggctggtccaaacctaactccaaactgcagataaataggcaagtcatagttgacagagataataggtccggttattctggtattttctctgttgaaggcaaaagctgcatcaatcggtgcttttatgtggagttgataaggggaaggaaccaggaaactgaagtcttgtggacctcaaacagtattgttgtgttttgtggcacctcaggtacatatggaacaggctcatggcctgatggggcggacatcaatctcatgcctatataa

SEQ ID NO: 12 - DNA, 1410 bp, unidentified
  misc_feature 1..1410: note = Deoptimized Influenza A virus
  source 1..1410: mol_type = other DNA
atgaaccctaatcaaaaaataattacaatcggatccgttagtctgacaatcgctactatatgttttctgactcagatagcgatactcgttacaaccgttacattgcatttcaaacaatacgaatgcaattccccccctaacaatcaggtaatgttgtgcgaacctacaataatcgaacggaatattaccgagatagtgtatctgactaatacgactatcgaaaaagagatatgcccaaaactagccgaatatcggaattggtcaaaaccgcaatgtaacataaccggattcgcaccattttcgaaagacaattcgattaggttgtccgccggaggcgatatttgggttacacgcgaaccttatgtgtcatgcgatcccgataaatgctatcaattcgcactcggacaggggactacccttaataacggacattctaacgataccgtacacgatagaactccatatcgaacattgctaatgaacgagttaggcgtaccattccatttgggcactaaacaggtatgtatcgcatggtctagctctagttgccatgacggtaaggcttggttgcatgtgtgcgttaccggcgacgataagaacgcaaccgctagctttatatataacggtaggttggtcgactcaatcgggtcatggtcaaaaaaaatacttagaacgcaagagtccgaatgcgtatgcataaacggtacatgcaccgtagtgatgaccgacggatccgctagcggtaaggccgatacgaaaatactgtttatcgaagagggtaagatagtgcatacgagtctactatccggatccgctcaacatgtcgaagagtgttcatgttatcctaggtatcccggcgttagatgcgtatgtagggataattggaaagggagtaatagacctatagtcgatattaacgttaaggattattcaatcgtaagtagttatgtgtgtagcggactcgtaggcgatacacctagaaaaaacgatagctctagtagctcacattgcctagaccctaataacgaagagggggggcatggcgttaagggatgggcattcgacgacggtaacgacgtttggatgggtaggactattagcgaaaagcttagatccgggtatgagacattcaaagtgatagagggatggtctaaacctaattcaaaactgcaaattaataggcaagtgatagtcgatagggataatagatccgggtattccggaatttttagcgttgagggtaagtcatgtattaataggtgtttttatgtcgagcttattagggggagaaatcaggaaaccgaagtgttgtggacatccaattcaatcgtcgttttttgcggaactagcggaacatacggtaccggatcatggcccgacggagccgatattaaccttatgcctatataa

SEQ ID NO: 13 - DNA, 1689 bp, Influenza A virus
  source 1..1689: mol_type = unassigned DNA
atggccatcatttatctcattctcctgttcacagcagtgagaggggaccagatatgcattggataccatgccaataattccacagagaaggtcgacacaattctagagcggaacgtcactgtgactcatgccaaggacattcttgagaagacccataacggaaagttatgcaaactaaacggaatccctccacttgaactaggggactgtagcattgccggatggctccttggaaatccagaatgtgataggcttctaagtgtgccagaatggtcctatataatggagaaagaaaacccgagagacggtttgtgttatccaggcagcttcaatgattatgaagaattgaaacatctcctcagcagcgtgaaacatttcgagaaagtaaagattctgcccaaagatagatggacacagcatacaacaactggaggttcacgggcctgcgcggtgtctggtaatccatcattcttcaggaacatggtctggctgacaaagaaaggatcaaattatccggttgccaaaggatcgtacaacaatacaagcggagaacaaatgctaataatttggggggtgcaccatcccaatgatgagacagaacaaagaacattgtaccagaatgtgggaacctatgtttccgtaggcacaccaacattgaacaaaaggtcaaccccagacatagcaacaaggcctaaagtgaatggacaaggaggtagaatggaattctcttggaccctattggatatgtgggacaccataaattttgagagtactggtaatctaattgcaccagagtatggattcaaaatatcgaaaagaggtagttcagggatcatgaaaacagaaggaacacttgggaactgtgagaccaaatgccaaactcctttgggagcaataaatacaacattgccttttcacaatgtccacccactgacaataggtgagtgccccaaatatgtaaaatcggagaagttggtcttagcaacaggactaaggaatgttccccagattgaatcaagaggattgtttggggcaatagctggttttatagaaggaggatggcaaggaatggttgatggttggtatggataccatcacagcaatgaccagggatcagggtatgcagcagacaaagaatccactcaaaaggcatttgatggaatcaccaacaaggtaaattctgtgattgaaaagatgaatacccaatttgaagctgttgggaaagaattcagtaacttagagagaagactggagaacttgaacaaaaagatggaagacgggtttctagatgtgtggacatacaatgctgagcttctagttctgatggaaaatgagaggacacttgactttcatgattctaatgtcaagaatctgtatgataaagtcagaatgcagctgagagacaacgtcaaagaactaggaaatggatgttttgaattttatcacaaatgtgatgatgaatgcatgaatagtgtgaaaaacgggacgtatgattatcccaagtatgaagaagagtctaaactaaatagaaatgaaatcaaaggggtaaaattgagcagcatgggggtttatcaaatccttgccatttatgctacagtagcaggttctctgtcactggcaatcatgatggctgggatctctttctggatgtgctccaacgggtctctgcagtgcaggatctgcatatga

SEQ ID NO: 14 - DNA, 1689 bp, unidentified
  misc_feature 1..1689: note = Deoptimized Influenza A virus
  source 1..1689: mol_type = other DNA
atggcaataatctatctgatactgttgtttacagccgttaggggcgatcagatatgcatagggtatcacgctaataatagtaccgaaaaagtcgatacaatactcgaaagaaacgtaaccgttacacacgctaaagatatactcgaaaagacacataacggtaagctatgcaaacttaacggtataccaccacttgagttaggcgattgctcaatcgcaggatggttgttggggaatcccgaatgcgataggctattgagcgtacccgaatggtcttatattatggaaaaagagaatcctagagacggattgtgttatcccggatcttttaacgattacgaagagcttaaacatctgctatctagcgttaaacatttcgaaaaagtgaaaattctgccaaaagataggtggacacagcatacgactaccggaggatctagggcatgcgccgttagcggtaatccgtcattctttagaaatatggtatggttgacaaaaaaggggtctaattatccagtcgctaagggatcgtataataatacaagcggagagcaaatgttgattatatggggagtgcatcaccctaacgacgaaaccgaacaacggacactgtatcaaaacgtcggaacatacgttagcgtcggtacaccaactctgaataaaagatcgactcccgatatcgcaactagaccaaaagtgaacggacagggggggagaatggagtttagttggacactactcgatatgtgggatacaattaatttcgaatcaaccggtaatctgatcgcacccgaatacgggtttaagattagtaaaagggggtcatccggtattatgaaaaccgaaggtacactagggaattgcgaaactaagtgtcagacaccactaggggctattaatacaacactaccatttcataatgtgcatccattgacaatcggagagtgtcctaagtatgtgaaatccgaaaaactagtgcttgcaaccggactgagaaacgtaccgcaaatcgaatccagagggttgttcggagcaatcgcagggtttatcgaaggggggtggcagggaatggtcgacggatggtatgggtatcatcactctaacgatcagggatccggatacgcagccgataaggagtcaacccaaaaagcattcgacggaattactaataaggtgaatagcgtaatcgaaaaaatgaatacacaattcgaagccgtcggtaaagagttttcgaatctcgaaaggagacttgagaatctgaataaaaaaatggaggacggattcttagacgtatggacatataatgccgaactgttagtccttatggagaacgaacggacactagactttcacgatagtaacgttaagaatctgtatgacaaagtgagaatgcaattgagagacaatgtgaaagagctaggtaacggatgtttcgaattctatcataaatgcgacgacgagtgtatgaatagcgttaaaaacggtacatatgactatcctaagtatgaggaagagtcaaagcttaatagaaacgagattaagggagtgaaactatctagtatgggagtgtatcagatactcgcaatatacgctacagtcgccggatccctatcacttgcgattatgatggccggaattagcttttggatgtgctctaacggatcattgcaatgtaggatttgcatatga

SEQ ID NO: 15 - DNA, 1410 bp, Influenza A virus
  source 1..1410: mol_type = unassigned DNA
atgaatccaaatcaaaagataataacaattggctctgtctctctcaccattgcaacagtatgcttcatcatgcagattgccatcctggcaactactgtgacattgcattttaaacaacatgagtgcgactcccccgcgagcaaccaagtaatgccatgtgaaccaataataatagaaaggaacataacagagatagtgtatttgaataacaccaccatagagaaagagatttgccccgaagcagtggaatacagaaattggtcaaagccgcaatgtcaaattacaggatttgcacctttttctaaggacaattcaatccggctttctgctggtggggacatttgggtgacgagagaaccttatgtgtcatgcgatcctggcaagtgttatcaatttgcactcgggcaggggaccacactagacaacaaacattcaaatggcacaatacatgatagaatccctcaccgaaccctattaatgaatgagttgggtgttccatttcatttaggaaccaaacaagtgtgtgtagcatggtccagctcaagttgtcacgatggaaaagcatggttgcatgtttgtgtcactggggatgatagaaatgcgactgctagcttcatttatgacgggaggcttgtggacagtattggttcatggtctcaaaatatcctcaggacccaggagtcggaatgcgtttgtatcaatgggacttgcacagtagtaatgactgatggaagtgcatcaggaagagccgatactagaatactattcattaaagaggggaaaattgtccatattagcccattgtcaggaagtgctcagcatatagaggagtgttcctgttaccctcgatatcctgacgtcagatgtatctgcagagacaactggaaaggctctaataggcccgttatagacataaatatggaagattatagcattgattccagttatgtgtgctcagggcttgttggcgacacacccaggaacgacgacagctctagcaatagcaattgcagggatcctaacaatgagagagggaatccaggagtgaaaggctgggcctttgacaatggagatgatgtatggatgggaagaacaatcaacaaagattcacgctcaggttatgaaactttcaaagtcattggtggttggtccacacctaattccaaatcgcaggtcaatagacaggtcatagttgacaacaataattggtctggttactctggtattttctctgttgagggcaaaagctgcatcaataggtgcttttatgtggagttgataaggggaaggccacaggagactagagtatggtggacctcaaacagtattgttgtgttttgtggcacttcaggtacttatggaacaggctcatggcctgatggggcgaacatcaatttcatgcctatataa

SEQ ID NO: 16 - DNA, 1410 bp, unidentified
  misc_feature 1..1410: note = Deoptimized Influenza A virus
  source 1..1410: mol_type = other DNA
atgaatcctaaccagaaaattattactatagggtcagtgtcattgactatcgcaaccgtatgctttattatgcaaatagcgatactcgcaactaccgtaacattgcattttaaacaacacgaatgcgatagtcccgctagcaatcaggtaatgccatgcgaacctattataatcgaacggaatattaccgagatagtgtatcttaacaatactactatcgaaaaagagatatgcccagaggccgtcgagtatagaaattggtctaaacctcaatgtcagattaccggattcgcaccattctctaaagacaattcgattagattgtccgccggaggcgatatatgggtgacacgcgaaccttatgtgtcatgcgatcccggtaagtgttatcaattcgcactcggacaggggactacactcgataataaacattctaacggtacgatacacgataggattccacataggacactattgatgaacgagttaggcgtaccgtttcatctaggcactaaacaggtatgcgttgcgtggtctagctcatcatgtcatgacggtaaggcatggttgcatgtgtgcgtaaccggcgacgatagaaacgctaccgctagttttatatacgacggtaggctagtcgattcaatcggatcatggtcacagaatatacttagaacacaggaatccgaatgcgtttgtattaacggtacatgtacagtcgttatgaccgacggatccgcatccggtagggccgatactaggatactgtttataaaagagggcaaaatcgtgcatattagcccacttagcggatccgcacaacatatcgaagagtgtagttgctatcctaggtatcctgacgttagatgtatttgcagagacaattggaaagggtctaatagacccgtaatcgatatcaatatggaggattattcaatcgatagctcttatgtgtgtagcggattagtcggcgatacacctagaaacgacgatagctctagtaattcgaattgtagggaccctaataacgagagaggcaatcccggcgttaaagggtgggcattcgataacggcgacgacgtttggatggggcgaacaattaataaggactctagatccgggtatgagacattcaaagtgataggggggtggtctacacctaactcaaaatctcaagtgaataggcaagtgatagtcgacaataacaattggtcagggtatagcggtatattctcagtcgagggtaagtcatgtattaatagatgtttttacgttgagttgattagggggcgaccacaagagactagagtgtggtggactagtaatagtatagtcgttttttgcggaactagcggtacatacggaaccggatcatggcctgacggagcgaatattaattttatgccaatctaa

SEQ ID NO: 17 - DNA, 1701 bp, Influenza A virus
  source 1..1701: mol_type = unassigned DNA
atgaagactatcattgctttgagctacattctatgtctggttttcgctcaaaaacttcccggaaatgacaacagcacggcaacgctgtgccttgggcaccatgcagtaccaaacggaacgatagtgaaaacaatcacgaatgaccaaattgaagttactaatgctactgagctggttcagagttcctcaacaggtgaaatatgcgacagtcctcatcagatccttgatggagaaaactgcacactaatagatgctctattgggagaccctcagtgtgatggcttccaaaataagaaatgggacctttttgttgaacgcagcaaagcctacagcaactgttacccttatgatgtgccggattatgcctcccttaggtcactagttgcctcatccggcacactggagtttaacaatgaaagcttcaattggactggagtcactcaaaatggaacaagctctgcttgcaaaaggagatctaataacagtttctttagtagactgaattggttgacccacttaaaattcaaatacccagcattgaacgtgactatgccaaacaatgaaaaatttgacaaattgtacatttggggggttcaccacccgggtacggacaatgaccaaatcttcttgtatgctcaagcatcaggaagaatcacagtctctaccaaaagaagccaacaaactgtaatcccgaatatcggatccagacctagagtaaggratatccccagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaattctgaatgcatcactccaaatggaagcattcccaatgacaaaccatttcaaaatgtaaacagaatcacatatggggcctgtcccagatatgttaagcaaaacactctgaaattggcaacagggatgagaaatgtaccagagaaacaaactagaggcatatttggcgcaatcgcgggtttcatagaaaatggttgggagggaatggtggatggttggtacggtttcaggcatcaaaattctgagggaataggacaagcagcagatctcaaaagcactcaagcagcaatcaatcaaatcaatgggaagctgaataggttgatcgggaaaaccaacgagaaattccatcagattgaaaaagaattctcagaagtagaagggagaattcaggacctcgagaaatatgttgaggacactaaaatagatctctggtcatacaacgcggagcttcttgttgccctggagaaccaacatacaattgatctaactgactcagaaatgaacaaactgtttgaaagaacaaagaagcaactgagggaaaatgctgaggatatgggcaatggttgtttcaaaatataccacaaatgtgacaatgcctgcataggatcaatcagaaatggaacttatgaccatgatgtatacagagatgaagcattaaacaaccggttccagatcaaaggcgttgagctgaagtcaggatacaaagattggatcctatggatttcctttgccatatcatgttttttgctttgtgttgttttgttggggttcatcatgtgggcctgccaaaaaggcaacattaggtgcaacatttgcatttga

SEQ ID NO: 18 - DNA, 1701 bp, unidentified
  misc_feature 1..1701: note = Deoptimized Influenza A virus
  source 1..1701: mol_type = other DNA
atgaaaacaattatcgcactgtcatacatactgtgtctggtattcgctcaaaaattgcccggtaacgacaattcaaccgctacattgtgcttagggcatcacgccgtaccgaacggaactatcgttaagacaattactaacgaccaaatcgaagtgactaacgctacagagttggtgcaatcctctagtacaggcgaaatatgcgattcaccacaccaaatccttgacggagagaattgtacacttatcgacgcactattaggcgatccacaatgcgacggatttcagaataaaaaatgggatctattcgttgagagatccaaagcttattcaaattgttatccatacgacgtaccggattacgctagccttaggtcactcgttgcgtcaagcggtactctcgaattcaataacgagtcattcaattggactggcgttacgcaaaacggaactagtagcgcatgtaaaagacggtctaataatagcttttttagcagactgaattggttgactcatctgaaattcaaatatcccgcacttaacgttactatgcctaataacgaaaaattcgataagctatatatatggggcgtacaccatcccggaacggataacgatcagatattcttgtacgctcaagctagcggtaggattaccgttagtactaaaagatcccaacaaaccgtaattccgaatatcggatctagacctagggtgagaratataccgtctaggattagcatatattggactatcgttaaacccggagacatactgttgatcaatagtacaggcaatctgatcgcacctagggggtatttcaaaattagatccggtaagtctagcattatgagatccgacgcaccaatcggtaaatgtaatagcgaatgcattacaccaaacggatcaatccctaacgataagccattccaaaacgtaaataggattacatacggcgcatgccctagatacgttaaacagaatacgcttaaacttgcgacaggtatgcgaaacgtacccgaaaaacagactagggggatattcggcgcaatcgccggatttatcgaaaacggatgggagggtatggtcgacggatggtacggatttagacatcaaaatagcgaagggatagggcaagccgccgatctgaaatcaacgcaagccgctattaatcaaattaacggaaaactgaatagattgatcggtaagactaacgaaaaatttcaccaaatcgaaaaagagtttagcgaagttgagggaaggatacaagaccttgagaaatacgttgaggatactaagatcgacctatggtcatataatgccgagttgctagtcgcactcgagaatcagcatacaatcgatctgactgatagcgaaatgaataaattgttcgaaagaacgaaaaaacaattgcgcgaaaacgccgaagacatggggaatgggtgttttaagatataccataaatgcgataacgcatgcatagggtcaatcagaaacggaacatacgatcacgacgtatatagagacgaagcccttaataatagattccaaattaaaggcgttgagcttaaaagcggatacaaagactggatactgtggattagtttcgcaatctcatgctttctattgtgcgttgtgctattggggttcataatgtgggcatgtcagaaagggaatattagatgcaatatttgtatatga

SEQ ID NO: 19 - DNA, 1410 bp, Influenza A virus
  source 1..1410: mol_type = unassigned DNA
atgaatccaaatcaaaagataataacgattggctctgtttctctcaccatttccacaatatgcttcttcatgcaaattgccatcttgataactactgtaacattgcatttcaagcaatatgaattcaactcccccccaaacaaccaagtgatgctgtgtgaaccaacaataatagaaagaaacataacagagatagtgtatctgaccaacaccaccatagagaaggaaatatgccccaaactagcagaatacagaaattggtcaaagccgcaatgtgacattacaggatttgcacctttttctaaggacaattcgattaggctttccgctggtggggacatctgggtgacaagagaaccttatgtgtcatgcgatcctgacaagtgttatcaatttgcccttggacagggaacaacactaaacaacgtgcattcaaacgacacagtacatgataggaccccttatcggaccctattgatgaatgagttaggtgttccatttcatctggggaccaagcaagtgtgcatagcatggtccagctcaagttgtcacgatggaaaagcatggctgcatgtttgtgtaacgggggatgataaaaatgcaactgctagcttcatttacaatgggaggcttgtagatagtgttgtttcatggtccaaagatatcctcaggacccaggagtcagaatgcgtttgtatcaatggaacttgtacagtagtaatgactgatgggagtgcttcaggaaaagctgatactaaaatactattcattgaggaggggaaaatcgttcatactagcacattgtcaggaagtgctcagcatgtcgaggagtgctcctgctatcctcgatatcctggtgtcagatgtgtctgcagagacaactggaaaggctccaataggcccattgtagatataaacataaagaattatagcattgtttccagttatgtgtgctcaggacttgttggagacacacccagaaaaaccgacagctccagcagtagccattgcttggatcctaacaatgaagaaggtggtcatggagtgaaaggctgggcctttgatgatggaaatgacgtgtggatgggaagaacgatcagcgagaagttacgcttaggatatgaaaccttcaaagtcattgaaggctggtccaaccctaattccaaattgcagataaataggcaagtcatagttgacagaggtaataggtccggttattctggtattttctctgttgaaggcaaaagctgcatcaatcggtgcttttatgtggagttgataaggggaagaaaagaggaaactgaagtcttgtggacctcaaacagtattgttgtattttgtggaacctcaggtacatatggaacaggctcatggcctgatggggcggacatcaatctcatgcctatataa

SEQ ID NO: 20 - DNA, 1410 bp, unidentified
  misc_feature 1..1410: note = Deoptimized Influenza A virus
  source 1..1410: mol_type = other DNA
atgaatcctaaccaaaagattattacaatcggatccgttagccttactatatccacaatttgtttttttatgcaaatagcgatactgataactaccgttacattgcatttcaaacaatacgaattcaattcaccccctaataatcaggttatgttgtgcgaacctactattatcgaacggaatataaccgagatagtgtatctaacgaacactacaatcgaaaaagagatatgccctaagctcgcagagtatagaaattggtcaaaaccccaatgcgatataaccggattcgcaccatttagtaaggataatagtattaggttgtccgccggaggcgatatatgggttacacgcgaaccatacgtgtcatgcgatcccgataaatgctatcaattcgctctcggacagggaacgacattgaataacgtacattcaaacgataccgtacacgataggacaccttatagaacactattgatgaacgaactaggcgtacctttccatctcggaactaaacaggtttgtatcgcttggtctagtagctcatgccatgacggtaaggcatggttgcatgtgtgcgttaccggcgacgataaaaacgcaaccgctagtttcatatataacggtaggttagtcgatagcgtagtgagttggtctaaagacatactgcgaacacaggaatccgagtgcgtatgcataaacggtacatgtaccgtagtgatgaccgacggatccgctagcggtaaggccgatacgaaaatattgttcatagaggagggtaagatagtgcatacaagtacactatccggatccgctcaacatgtcgaagagtgctcatgttatcctagatatcccggcgttagatgcgtatgtagagacaattggaaagggtctaatagaccgatagtcgacattaatattaaaaactattcaatcgttagctcatatgtgtgttccggattagtcggcgatacccctagaaaaaccgatagctctagctcatcccattgtcttgaccctaataacgaagagggggggcatggcgttaagggatgggcattcgacgacggtaacgacgtttggatgggacggacaattagcgaaaaacttagattggggtatgagacttttaaggtaatcgaagggtggtctaatcctaattcgaaactgcaaattaataggcaagtgatagtcgatagggggaataggtccggatatagcggaatcttttccgttgagggtaagtcatgtattaataggtgtttttatgtcgaactgattagggggagaaaagaggaaaccgaagtgttatggactagtaactcaatcgttgtgttttgcggtacatccggtacttatggaaccggatcatggccagacggagccgatataaaccttatgccaatttaa

SEQ ID NO: 21 - DNA, 1704 bp, Influenza A virus
  source 1..1704: mol_type = unassigned DNA
atggagaaaatagtgcttcttcttgcaatagtcagccttgttaaaagtgatcagatttgcatcggttaccatgcaaacaactcgacagagcaggttgacacaataatggaaaagaacgttactgttacacatgcccaagacatactggagaagacacataacgggaaactctgcgatctagatggagtgaagcctctgattctacgagattgtagtgtagctggatggctcctcggaaacccaatgtgtgacgaattcatcaatgtgccggaatggtcttacatagtggagaaggccaacccagccaatgacctctgttacccagggaatttcaacgactatgaagaactgaaacacctattgagcagaataaaccattttgagaaaattcagatcatccccaaaagttcttggtccgatcatgaagcctcatcaggggtgagctcagcatgtccataccagggaacgccctcctttttcagaaatgtggtatggcttatcaaaaagaacaatacatacccaacaataaagagaagctacaataataccaaccaggaaaatcttttgatactgtgggggattcatcattctaatgatgcagcagagcagataaagctctatcaaaacccaaccacctatatttccgttgggacatcaacactaaaccagagattggtaccaaaaatagccactagatccaaagtaaacgggcaaagtggaaggatggatttcttctggacaattttaaaaccgaatgatgcaatcaacttcgagagtaatggaaatttcattgctccagaatatgcatacaaaattgtcaaggaaggagactcagcaattatgaaaagtgaagtggaatatggtaactgcaacaccaagtgtcaaactccaataggggcgataaactctagtatgccattccacaacatacaccctctcaccatcggggaatgccccaaatatgtgaaatcaaacaaattagtccttgctactgggctcagaaatagtcctctaagagaaagaagaagaaaaagaggactatttggagctatagcagggtttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaataacttggaaaggagaatagagaacttaaacaagaaaatggaagacggattcctagatgtctggacttataatgctgaacttctggttctcatggaaaatgagagaactctagacttccatgactcaaatgtcaagaacctttacgacagggtccgactacagcttagggataatgcaaaggagctgggtaacggttgtttcgagttctatcacaaatgtgataatgaatgtatggaaagtgtaagaaacggaacgtatgactacccgcagtattcagaagaagcaagattaaaaagagaggaaataagtggagtaaaattggaatcaatgggaacttaccaaatactgtcaatttattcaacagttgcgagttctctagcactggcaatcatggtggctggtctatctttgtggatgtgctccaatgggtcgttacaatgcagaatttgcatttaa

SEQ ID NO: 22 - DNA, 1704 bp, unidentified
  misc_feature 1..1704: note = Deoptimized Influenza A virus
  source 1..1704: mol_type = other DNA
atggagaaaatagtgctactactcgcaatcgttagtctggttaagtccgatcagatatgcatagggtatcacgctaacaatagtaccgaacaggtcgacactattatggaaaaaaacgttaccgttacacacgcacaggacatactcgaaaaaacccataacggtaagttatgcgatttagacggagttaagccactgatacttagggattgttcagtcgccggatggttgttagggaatccaatgtgcgacgaattcattaacgtacccgaatggtcatacatagtcgaaaaagcgaatcccgctaacgatctatgttatccagggaattttaacgattacgaagagcttaagcatctactatctagaataaaccatttcgaaaagattcagataataccgaaatcgagttggtccgatcacgaagcgtcaagcggagtgagtagcgcatgcccataccaaggaacaccatcattctttagaaacgtcgtttggttgattaaaaaaaataatacatatccgactattaagagatcatataataatacaaaccaagagaatctactgatactatgggggatacaccatagtaacgacgcagccgaacagattaagctatatcagaatccaactacatacattagcgtagggactagtacacttaatcagagactcgtacctaaaatcgctactagatcgaaggtaaacggacaatccggtagaatggactttttttggactatactgaaacctaacgacgcaattaatttcgaatctaacggaaattttatcgctcccgaatacgcatataagatagtgaaagagggggatagcgcaattatgaaatccgaagtcgaatacggaaattgcaatactaagtgtcagacaccaatcggagcaattaactctagtatgccattccataacatacatccacttacaatcggagaatgccctaaatacgttaagtctaacaaactcgtactcgcaaccggacttaggaatagtccacttagagagagacgaagaaagagagggttgttcggagcaatcgcagggttcatagagggggggtggcagggtatggtcgacggatggtacgggtatcatcattctaacgaacagggatccggatacgcagccgataaagagagtactcagaaagcaatcgacggagtgacgaataaagtgaattcgataatcgataagatgaatacgcaattcgaagccgtaggtagggaattcaataatctcgagagacgaatcgaaaaccttaacaaaaaaatggaagacggattcctagacgtatggacttataacgccgaactgttagtgcttatggagaacgaaagaacccttgactttcacgattctaacgttaagaatctatacgatagagtgagactgcaattgagggataacgctaaagagttagggaacgggtgtttcgaattctatcataaatgcgataacgaatgtatggagtcagtgagaaacggtacatacgactatccgcaatattccgaagaggctagattgaaaagagaggagattagcggagtgaaacttgagtcaatggggacatatcagatattgtcaatatactcaaccgtcgctagtagtctcgcactcgcaattatggtcgccggactgtcactatggatgtgttcaaacggtagtctgcaatgtaggatttgtatataa

SEQ ID NO: 23 - DNA, 1350 bp, Influenza A virus
  source 1..1350: mol_type = unassigned DNA
atgaatccaaatcagaagataataaccattgggtcaatctgtatggtaattggaatagttagcttaatgttacaaattgggaacataatctcaatatgggtcagtcattcaattcaaacagggaatcaacaccaagatgaaccaatcagaaatgctaattttcttactgagaacgctgtggcttcagtaacattagcgggcaattcatctctttgccccgttagaggatgggctgtacacagtaaagacaacagtataaggattggttccaagggggatgtgtttgttattagagagccgttcatctcatgctcccacttggaatgcagaactttctttttgactcagggagccttactgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggcccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtaatgggcaggcatcatataagatcttcaaaatggaaaaaggaaaagtggttaaatcagtcgaattgaatgcccctaattatcactatgaggaatgctcctgttatcctgatgctggcgaaatcacatgtgtgtgcagggataattggcatggctcaaataggccatgggtatctttcaatcagaatttggagtatcaaataggatatatatgtagtggagttttcggagacaatccacgccccaatgatggaacaggtagttgtgatccagtgtcccctaacggggcatatgggataaaagggttttcatttaaatacggcaatggtgtttggatcggaagaaccaaaagcactaattccaggagtggttttgaaatgatttgggatccaaatgggtggactgaaacggacagtagcttttcagtgaaacaagatatagtagcaataactgattggtcagggtatagcgggagttttgttcagcatccagaactgacaggattagattgcataagaccttgcttctgggttgagttaatcagagggcggcccaaagagagcacaatttggactagtgggagcagcatatctttttgtggtgtaaatagcgacactgtgagttggtcttggccagacggtgctgagttgccattcaccattgacaagtag

SEQ ID NO: 24 - DNA, 1350 bp, unidentified
  misc_feature 1..1350: note = Deoptimized Influenza A virus
  source 1..1350: mol_type = other DNA
atgaatccgaatcaaaaaattataacaatagggtcaatctgtatggtaatcggtatagtgtcacttatgttacaaatcgggaatattatatctatttgggtgtcacactcaatccaaaccggtaatcaacaccaagacgaacctatacggaatgcgaatttcttaacagagaatgccgtagctagcgttacgttagccggtaatagttcattgtgtcccgttagggggtgggctgtgcatagtaaggataatagtattaggatagggtctaaaggcgacgtattcgtgatacgcgaaccttttatctcttgctcacacttagagtgtagaacattttttctgactcaaggcgcactgttaaacgataaacactctaacggtacagttaaggataggtcaccacataggacattgatgtcatgtcccgtaggcgaagctcctagtccatataatagtagattcgaaagcgttgcatggtccgctagcgcttgtcacgacggaactagttggttgacaatcgggatatccggacccgataatggcgcagtcgcagtgttgaagtataatgggattataaccgatactatcaaatcatggagaaataatatactgagaacacaggagtccgaatgcgcttgcgttaacggatcatgctttaccgttatgactgacggaccatctaacgggcaagctagttataaaattttcaaaatggagaaaggtaaggtagtgaaatccgttgagcttaacgctccaaattatcattacgaagagtgtagttgctatccagacgctggcgaaattacttgcgtatgtagagacaattggcacggatctaatagaccatgggttagctttaatcagaatttagagtatcagatagggtatatatgttccggagtgttcggcgataatcctagacctaacgacggtacagggtcatgcgatccagtgagtccaaacggcgcatacggaattaaagggtttagctttaagtatgggaatggcgtatggatcggtaggactaagtctactaatagtagatccggattcgaaatgatatgggaccctaatgggtggactgagactgatagtagttttagcgtaaaacaggatatagtcgctataaccgattggagcgggtatagcggatcattcgtacagcatcccgaattgactgggttagactgtattagaccatgcttttgggtcgaattgattagggggagaccaaaagagtcaactatatggactagcggatctagtattagtttttgcggagtgaattccgataccgttagttggtcatggccagacggagctgagttgccatttacaatcgataaatag

SEQ ID NO: 25 - DNA, 1659 bp, Influenza A virus
  source 1..1659: mol_type = unassigned DNA
atgaacattcaaattctggcattcattgcttgtgtgctgactggagctaaaggagacaaaatatgtcttgggcaccatgctgtggcaaatggaacaaaagtgaacacattaacagagagggggattgaagtagtgaatgccacagagacagttgaaactgcgaatatcaagaaaatatgtactcaagggaaaagaccaacagatctgggacaatgtggacttctagggaccctaataggacctccccaatgtgatcaattcctggagttttcctctgatttgataattgagcgaagagaaggaaccgatgtatgctatcccggtaaattcacaaatgaagaatcactgagacagatccttcgaagatcaggaggaattggtaaggagtcaatgggcttcacctatagtggaataaggaccaatggagcgacaagtgcctgcacaagatcaggttcttctttctatgcagagatgaagtggttgctgtcgaattcagacaatgcagcattcccacagatgacaaaatcgtatagaaatcccagaaacaaaccagctctgataatttggggagttcatcactctgaatcggttagcgagcagaccaaactctatggaagtggaaacaagttgataaaagtaagaagctcaaaataccaacaatcatttaccccaaatcctggagcacggagaatcgatttccactggctactcctggatcccaatgacacagtgaccttcactttcaatggggcattcatagcccctgacagggcaagtttctttagaggagaatcaataggagtccagagtgatgctcctttggattctagttgtggagggaattgctttcacagtgggggtacgatagtcagttccctgccattccaaaacatcaaccctagaactgtgggaaaatgccctcggtatgtcaaacagaaaagcctccttctggctacaggaatgagaaatgttccagagaaaccaaagaaaagaggcctttttggagcaattgctggattcatagagaacggatgggagggtctcatcaatggatggtatggtttcagacatcaaaatgcacaaggagagggaactgcagctgactacaaaagcacccagtctgcaatagatcagatcacaggcaaattgaatcgtctaattggcaaaacaaatcagcagtttgggctgatagacaatgagttcaatgaggtagaacaacaaataggaaatgtcattaattggacacaagacgcaatgactgagatatggtcgtataatgctgagctgttggtggcaatggaaaatcaacatacaatagatcttacggattcagaaatgagcaaactttatgagcgtgtcagaaaacaactgagggagaatgctgaagaagatgggactggatgtttcgaaatattccataagtgtgatgatcattgtatggagagcataagaaacaacacttatgaccatactcaatacagaacagagtcactgcagaatagaatacagatagacccagtgaaattgagtagtggatacaaagacataatcttatggtttagcttcggggcatcatgttttcttcttctagccattgcaatgggattggttttcatttgcataaaaaatggaaacatgcagtgcactatttgtatatag

SEQ ID NO: 26 - DNA, 1659 bp, unidentified
  misc_feature 1..1659: note = Deoptimized Influenza A virus
  source 1..1659: mol_type = other DNA
atgaatatacagatactcgcattcatagcttgcgtacttaccggagctaaaggcgataagatatgtctagggcatcacgcagtcgcaaacggaacgaaagtgaatacacttacagagagagggatagaggtcgttaacgctacagagacagtcgaaaccgcaaatattaaaaaaatttgtacacaaggaaaacgaccaaccgatctgggacaatgcggactgttagggacactgataggaccaccacaatgcgatcaattccttgagtttagtagcgatctgataatcgaacgaagagagggaactgacgtttgttatcccggtaagttcactaacgaagagagtcttagacagatactgagacggtcagggggaatcggaaaagagtcaatggggtttacgtattctgggattaggactaatggcgcaactagcgcatgtactagaagcggatcatcattctatgccgaaatgaaatggttgttgtcgaattccgataacgctgcattcccacaaatgactaaatcgtatagaaatcctaggaataaacccgcactgataatatggggagtgcatcatagcgaatccgtaagtgaacagactaaattgtacggatcaggtaataaactgattaaagtgagatctagtaagtatcagcaatcgtttacacctaatcccggagctagacgtatcgatttccattggctattgctcgaccctaacgataccgttacattcacattcaatggcgcattcatagcgccagatagggcaagtttttttagaggcgaatcaatcggagtgcaatcagacgcaccacttgactcaagttgcggagggaattgtttccatagcggagggactatagtgagtagtctgccattccaaaatattaatcctagaacagtgggtaagtgtcctagatacgttaaacagaaaagtctgttactcgcaaccggaatgcgtaacgtacccgaaaaacctaaaaaaaggggattgttcggagcgatagccggattcatagagaatggatgggagggactgattaacggatggtacggatttagacaccaaaacgctcagggagagggaaccgcagccgattataaatcgacacaatctgcaatcgatcagattaccggtaagcttaatagattgattggtaagactaatcagcaattcggactgatagacaatgagtttaacgaagtcgagcaacagatagggaatgtgattaattggacacaagacgctatgactgagatttggtcttataatgccgaactgctagtcgctatggagaatcaacacacaatcgatctaaccgatagcgaaatgtcaaaattgtatgagagagtgagaaaacagcttagagagaatgcagaggaagacggaactgggtgtttcgagatattccataaatgcgacgatcactgtatggaatctattagaaataatacatacgatcatacacagtatagaacagagtcacttcaaaatcggatacagatagacccagttaaactatctagcggatataaagacataatactgtggttctcattcggagctagttgttttctgttgctcgcaatcgctatgggacttgtattcatatgtattaaaaacggtaatatgcaatgtacaatttgcatatag

SEQ ID NO: 27 - DNA, 1362 bp, Influenza A virus
  source 1..1362: mol_type = unassigned DNA
atgaatccaaatcagaagataataacaattggctccgtctctctaaccattgcaacagtatgtttcctcatgcagattgccattctagcaatgactgtaacactgcatttcaggcaaaatgaatgcagcatttccgcgaacagtcaggtagtgccgtgtgaaccaactacagagaaagaggtctgttcgaacgtagtagactatagaagctggtcaaagccgcagtgtcaaattacaggatttgcccctttttccaaggacaactcaattcgactttctgctggtggagacatttggataacaagagagccttatgtgtcgtgtgacaccagcaaatgttaccaatttgcacttgggcaggggaccacactggataacaaacattcaaacggaacaatacatgatagaatctcccatcggacccttttgatgaatgaactgggtgttccatttcacttgggaaccaaacaagtttgcatagcatggtccagctcaagttgccatgatgggaaagcatggttgcacgtttgtgtcactggggatgatagaaatgcaactgctagtttcatttacaatgggatgcttgttgacagtattggttcatggtctcaaaatatcctcaggacccaggagtcagaatgcgtttgcatcaatgggtcttgtacagtagtgatgactgatggaagtgcctcagggaaggccgatactaggatattattcgtcaaagaaggaaagattgttcacattagcccattgtcaggaagtgctcagcatatagaggaatgttcctgttatccccgatacccaaacgtcagatgtgtctgcagggacaactggaagggctctaataggcctgttatagacataaacatggcagattatagcatcgactccagttatgtgtgctcaggactcgttggggacacaccaaggaatgaggatagttctagcagcagcaactgtagggatcccaatgaagagaggggaaacccaggagtgaaaggatgggcctttgacagtggagatgatgtttggatgggtagaacaatcagtagggattcgcggtcaggctatgagacatttagggtcattggtggttggaccactgccaattccaaatcacagaccagcagacaagtcatagttgataataacaattggtctggttattctggtattttctctgttgaacacaaaagctgtatcaataggtgtttttatgtggagttaataagaggaaggccgaaagaaactagagtatggtggacctcaaacagtattgtcgtgttttgtggcacttctggcacttatggaacaggctcatggcctgatggggcgaacatcaatttcatgcctatataa

SEQ ID NO: 28 - DNA, 1362 bp, unidentified
  misc_feature 1..1362: note = Deoptimized Influenza A virus
  source 1..1362: mol_type = other DNA
atgaatccgaatcagaaaatcattactatcggatccgttagcttgacaatcgcaaccgtatgttttcttatgcagattgcgatactcgcaatgaccgttacattgcattttagacaaaacgagtgttctattagcgctaactctcaggtcgtgccatgcgaacctacaaccgaaaaagaggtttgttcaaacgtagtcgattataggtcatggtctaaaccgcaatgtcagattaccggattcgcaccattttcgaaagacaattcgattagactatccgccggaggcgatatttggataactagggaaccatacgtgtcatgcgatacaagtaagtgttatcaattcgcactcggccaagggactacactcgataacaaacactctaacggtacaatacacgataggattagtcataggacactgcttatgaacgagttaggcgtaccattccatctgggaactaaacaggtatgcatagcctggtcatctagttcatgtcacgacggtaaggcatggttgcacgtatgcgtaaccggcgacgatagaaacgctaccgcctcattcatatataacggtatgctagtcgactcaatcgggtcatggtcacaaaatatacttaggacacaggaatccgaatgcgtatgtattaacggatcatgtacagtcgttatgaccgacggatccgctagcggtaaggccgatacacggatactgttcgttaaagagggtaagatagtgcatattagcccacttagcggatccgcccaacatatcgaagagtgttcatgttatcctagatatccgaacgttaggtgcgtttgtagggataattggaaagggtctaatcgacccgttatcgatattaatatggccgattatagtatcgatagttcatacgtttgttccggattagtcggcgatactcctagaaacgaagatagttctagctctagtaattgtagagacccaaacgaagagagagggaatcccggagtgaaagggtgggcattcgatagcggtgacgacgtttggatgggtaggacaattagtagggactctagatccgggtatgagacttttagggtgataggcggatggacaaccgcaaactctaagagtcagactagtagacaggtgatagtcgataataataattggtccgggtatagcgggatttttagcgtcgagcataagtcatgtattaatcggtgtttttatgtcgaattgattagggggcgacctaaagagactagggtgtggtggactagcaattcgatagtcgttttttgcggtactagcggaacatacggaaccggaagttggccagacggagcgaatattaattttatgcctatataa

SEQ ID NO: 29 - DNA, 1704 bp, Influenza A virus
  source 1..1704: mol_type = unassigned DNA
atgaatactcaaattttggcattcattgcttgtatgctgattggaactaaaggagacaaaatatgtcttgggcaccatgctgtggcaaatgggacaaaagtgaacacactaacagagaggggaattgaagtagtcaatgccacggagacggtggaaactgtaaatattaagaaaatatgcactcaaggaaaaaggccaacagatctgggacaatgtggacttctaggaaccctaataggacctccccaatgcgatcaatttctggagtttgacgctaatttgataattgaacgaagagaaggaaccgatgtgtgctatcccgggaagttcacaaatgaagaatcactgaggcagatccttcgagggtcaggaggaattgataaagagtcaatgggtttcacctatagtggaataagaaccaatggggcgacgagtgcctgcagaagatcaggttcttctttctatgcggagatgaaatggttactgtcgaattcagacaatgcggcatttccccaaatgactaagtcgtataggaatcccaggaacaaaccagctctgataatctggggagtgcatcactctggatcagctactgagcagaccaaactctatggaagtggaaacaagttgataacagtaggaagctcgaaataccagcaatcattcactccaagtccgggagcacggccacaagtgaatggacaatcaggaaggattgattttcattggctactccttgaccccaatgacacagtgaccttcactttcaatggggcattcatagcccctgacagggcaagtttctttagaggagaatcgctaggagtccagagtgatgttcctttggattctggttgtgaaggggattgcttccacagtgggggtacgatagtcagttccctgccattccaaaacatcaaccctagaacagtggggaaatgccctcgatatgtcaaacagacaagcctccttttggctacaggaatgagaaacgtcccagagaaccccaagcaggcctaccagaaacggatgaccagaggcctttttggagcgattgctggattcatagagaatggatgggaaggtctcatcgatggatggtatggtttcagacatcaaaatgcacaaggagaaggaactgcagctgactacaaaagcacccaatctgcaatagatcagatcacaggcaaattgaatcgtctgattgacaaaacaaaccagcagtttgaactgatagacaatgaattcagtgagatagaacaacaaatcgggaatgtcattaactggacacgagactcaatgactgaggtatggtcgtataatgctgagctgttggtggcaatggagaatcagcatacaatagatcttgcagactcagaaatgaacaaactttacgaacgcgtcagaaaacaactaagggaaaatgctgaagaagatggaactggatgctttgagatattccataagtgtgatgatcagtgtatggagagcataaggaacaacacttatgaccatacccaatacaggacagagtcattgcagaatagaatacagatagacccagtgaaattgagtagtggatacaaagacataatcttatggtttagcttcggggcatcatgttttcttcttctagccattgcaatgggattggttttcatttgcataaagaatggaaacatgcggtgcactatttgtatatag

SEQ ID NO: 30 - DNA, 1704 bp, unidentified
  misc_feature 1..1704: note = Deoptimized Influenza A virus
  source 1..1704: mol_type = other DNA
atgaatacacagatactcgcattcatagcgtgtatgcttatcggaactaaaggcgataaaatttgcttagggcatcacgcagtcgctaacggaactaaagtgaatacgcttaccgaacgcggaatagaggtcgtgaacgctaccgagacagtcgaaacagtcaatataaaaaaaatttgtacacagggaaaaagaccaaccgatctgggacaatgcggactgttagggacactaatcggaccaccacaatgcgatcaattcctcgaattcgacgctaatctgataatcgaacggagagagggaactgacgtatgctatcccggtaagtttacgaacgaagagtcacttagacagatacttagggggtcaggggggatagacaaagagtctatggggtttacatatagcggaatacggactaacggagctacaagtgcatgtagacgatccggatcatcgttttacgccgaaatgaaatggttgttgtctaatagcgataacgctgcattcccacaaatgactaagtcttataggaatcctagaaataaacccgcactgattatttggggagtgcatcatagtggatcagcaaccgaacagactaagttgtacggatcaggtaataaactgattacagtcggatcgagtaaatatcagcaatcgttcacacctagtcccggagctagaccgcaagtgaacggacaatctggtaggattgactttcattggttgcttctagacccaaacgatacagtgacattcacttttaacggagcatttatcgcacccgatagggctagtttctttaggggagagtcactcggagtgcaatcagacgtaccacttgatagcggatgcgaaggcgattgttttcactcagggggaactatagtgagtagtctgccattccaaaatattaatcctagaaccgtcggtaagtgtcctaggtacgttaaacagactagtctattgctcgcaaccggaatgcgtaacgtacccgaaaatcctaaacaggcatatcagaaacggatgactagggggctattcggagcgattgccggattcatagagaatgggtgggagggactgatagacggatggtacgggttcagacaccaaaacgctcagggagagggaacagccgcagactataagtctacgcaatcggcaatcgatcagattaccggtaagcttaatagactgatagacaaaactaatcagcaattcgaactgatagacaacgaatttagtgagatagagcaacagatagggaatgtgataaattggactagagactcaatgactgaggtatggtcatataacgccgaactgttggtcgcaatggagaatcagcatacaatcgatctagccgatagcgaaatgaataaactttacgaaagggtgcgaaaacaattgcgagagaatgcggaagaggacggaaccggatgtttcgaaattttccataaatgcgacgatcaatgtatggaatcgattaggaataatacatacgatcatacacaatatagaaccgaatcacttcagaataggattcaaatcgatcccgttaagttgagtagcggatataaagacattatactatggttctcattcggagctagttgctttctattgcttgcgatagctatgggattggtgttcatatgcataaaaaacggtaatatgcgatgtacgatttgcatatag

SEQ ID NO: 31 - DNA, 1410 bp, Influenza A virus
  source 1..1410: mol_type = unassigned DNA
atgaatccgaatcagaagataataacaatcggggtagtgaataccactctgtcaacaatagcccttctcattggagtgggaaacttagttttcaacacagtcatacatgagaaaataggagaccatcaaatagtgacccatccaacaataatgacccctgaagtaccgaactgcagtgacactataataacatacaataacactgttataaacaacataacaacaacaataataactgaagcagaaaggcctttcaagtctccactaccgctgtgccccttcagaggattcttcccttttcacaaggacaatgcaatacgactgggtgaaaacaaagacgtcatagtcacaagggagccttatgttagctgcgataatgacaactgctggtcctttgctctcgcacaaggagcattgctagggactaaacatagcaatgggaccattaaagacagaacaccatataggtctctaattcgtttcccaataggaacagctccagtactaggaaattacaaagagatatgcattgcttggtcgagcagcagttgctttgacgggaaagagtggatgcatgtgtgcatgacagggaatgataatgatgcaagtgcccagataatatatggaggaagaatgacagactccattaaatcatggaggaaagacatactaagaacccaggagtctgaatgtcaatgcattgacgggacttgtgttgttgctgtcacagatggccctgctgctaatagtgcagatcacagggtttactggatacgggagggaagaataataaagtatgaaaatgttcccaaaacaaagatacaacacttagaagaatgttcctgctatgtggacattgatgtttactgtatatgtagggacaattggaagggctctaacagaccttggatgagaatcaacaacgagactatactggaaacaggatatgtatgtagtaaatttcactcagacacccccaggccagctgacccttcaataatgtcatgtgactccccaagcaatgtcaatggaggacccggagtgaaggggtttggtttcaaagctggcaatgatgtatggttaggtagaacagtgtcaactagtggtagatcgggctttgaaattatcaaagttacagaagggtggatcaactctcctaaccatgtcaaatcaattacacaaacactagtgtccaacaatgactggtcaggctattcaggtagcttcattgtcaaagccaaggactgttttcagccctgtttttatgttgagcttatacgagggaggcccaacaagaatgatgacgtctcttggacaagtaatagtatagttactttctgtggactagacaatgaacctggatcgggaaattggccagatggttctaacattgggtttatgcccaagtaa

SEQ ID NO: 32 - DNA, 1410 bp, unidentified
  misc_feature 1..1410: note = Deoptimized Influenza A virus
  source 1..1410: mol_type = other DNA
atgaatcctaatcagaaaataattactataggggtcgttaatactacactatctacaatcgctctactaatcggagtcggtaatctagtctttaatacagtgatacacgaaaagataggcgaccatcagatagtgacacatcctacaattatgacacccgaagtgcctaattgtagcgatacaataattacatataacaataccgttataaacaatattacaacaacaattataaccgaagccgaacgaccattcaaaagtccactacccctatgtccatttagggggttttttccgtttcataaggataacgctatacggttaggcgaaaataaagacgtaatcgttactagggagccatacgttagttgcgataacgataattgttggtcattcgcactcgctcaaggcgcactgttagggactaaacactctaacggaacaattaaagacagaacaccttataggtcactgataagattccctatcggaaccgctcccgtactaggcaattataaagagatatgcatagcatggtcaagttcgtcatgtttcgacggtaaagagtggatgcacgtatgtatgaccggtaacgataacgacgctagcgcacagataatatacggagggcgaatgacagactcaattaagagttggcgtaaagacatactgagaacacaagagtccgaatgccaatgcatagacggaacttgcgtagtcgccgttacagacggacccgcagctaactccgctgaccatagagtgtattggattagggagggaaggataataaagtatgagaacgtgcctaagactaagatacaacatcttgaagagtgttcatgttatgtcgacatagacgtgtattgcatatgtagagacaattggaaagggtctaataggccatggatgagaataaataacgaaactatactcgaaaccggatacgtatgttctaagttccatagcgatacacctagacccgcagacccatctattatgtcatgcgatagcccatctaacgttaacggcggacccggagtcaaagggttcggattcaaagccggtaacgacgtttggttagggagaaccgttagtactagcggtaggtccggattcgaaattataaaggttacagaggggtggataaatagtccgaatcacgttaagtcaattacacaaacacttgtgtctaataacgattggtccggatatagcggatcattcatagtcaaagctaaggattgctttcagccatgtttttacgtcgaactgataagggggagaccgaataaaaacgacgacgttagttggactagtaattcgatagtgacattttgcggattggacaacgaacccggatccggtaattggcctgacggatcgaatatagggtttatgcctaaataa

SEQ ID NO: 33 - DNA, 1710 bp, Influenza A virus
  source 1..1710: mol_type = unassigned DNA
agcaaaagcaggggatacaaaatgaacactcaaatcctggtattcgctctggtggcgagcattccgacaaatgcagacaagatctgccttgggcatcatgccgtgtcaaacgggactaaagtaaacacattaactgagagaggagtggaagtcgttaatgcaactgaaacggtggaacgaacaaacgttcccaggatctgctcaaaagggaaaaggacagttgacctcggtcaatgtggacttctgggaacaatcactgggccaccccaatgtgaccaattcctagaattttcggccgacttaattattgagaggcgagaaggaagtgatgtctgttatcctgggaaattcgtgaatgaagaagctctgaggcaaattctcagagagtcaggcggaattgacaaggagacaatgggattcacctacagcggaataagaactaatggaacaaccagtgcatgtaggagatcaggatcttcattctatgcagagatgaaatggctcctgtcaaacacagacaatgctgctttcccgcaaatgactaagtcatacaagaacacaaggaaagacccagctctgataatatgggggatccaccattccggatcaactacagaacagaccaagctatatgggagtggaaacaaactgataacagttgggagttctaattaccaacagtcctttgtaccgagtccaggagcgagaccacaagtgaatggccaatctggaagaattgactttcattggctgatactaaaccctaatgacacggtcactttcagtttcaatggggccttcatagctccagaccgtgcaagctttctgagagggaagtccatgggaattcagagtgaagtacaggttgatgccaattgtgaaggagattgctatcatagtggagggacaataataagtaatttgccctttcagaacataaatagcagggcagtaggaaaatgtccgagatatgttaagcaagagagtctgctgttggcaacaggaatgaagaatgttcccgaaatcccaaagaggaggaggagaggcctatttggtgctatagcgggtttcattgaaaatggatgggaaggtttgattgatgggtggtatggcttcaggcatcaaaatgcacaaggggagggaactgctgcagattacaaaagcacccaatcagcaattgatcaaataacagggaaattaaatcggcttatagaaaaaactaaccaacagtttgagttaatagacaacgaattcactgaggttgaaaggcaaattggcaatgtgataaactggaccagagattccatgacagaagtgtggtcctataacgctgaactcttagtagcaatggagaatcagcacacaattgatctggccgactcagaaatgaacaaactgtacgaacgagtgaagagacaactgagagagaatgccgaagaagatggcactggttgcttcgaaatatttcacaagtgtgatgacgactgcatggccagtattagaaacaacacctatgatcacagcaagtacagggaagaagcaatacaaaatagaatacagattgacccagtcaaactaagcagcggctacaaagatgtgatactttggtttagcttcggggcatcatgtttcatacttctggccattgcaatgggccttgtcttcatatgtgtgaagaatggaaacatgcggtgcactatttgtatataa

SEQ ID NO: 34 - DNA, 1710 bp, unidentified
  misc_feature 1..1710: note = Deoptimized Influenza A virus
  source 1..1710: mol_type = other DNA
agtaagagtagggggtataaaatgaatacacagatactcgtattcgcactcgttgcgtcaataccgacaaacgccgataagatttgcctagggcatcacgcagtgtcaaacggaactaaagtgaatacacttaccgaaaggggcgttgaggtagtgaacgctacagagactgtcgaacggactaacgtacctaggatttgtagtaagggtaaaagaacagtcgacctagggcaatgcggactgttaggcacaattaccggaccaccacaatgcgaccaatttctcgaatttagcgctgatctgattatcgaacggagagagggatccgacgtttgttatcccggtaaattcgttaacgaagaggcactgagacagatacttagagaatccggagggatagacaaagagacaatggggtttacatatagcggaattagaactaacggaactactagcgcatgtaggagatccggatctagcttttacgccgaaatgaaatggttactgtcaaataccgataacgccgcatttccgcaaatgactaagtcatataagaatactaggaaagaccccgcactgataatttgggggatacaccatagcggatcgactaccgaacagacaaagctatacggtagcgggaataaactgataacagtgggatcaagtaattaccaacagtcattcgtaccgagtccaggcgctagaccacaagtgaacggacaatccggacgtatagatttccattggttgatactgaatccgaacgatacagtgacatttagctttaacggcgcattcatagcacccgatagggcatcattccttaggggtaagagtatggggatacaaagcgaagtgcaagtcgacgctaattgcgaaggcgattgttatcatagcggggggactattattagtaatctgccattccaaaatattaatagtagggcagtgggaaagtgtccaaggtacgttaaacaggaatcactgttactcgcaaccggaatgaaaaacgtaccagagatacctaagagacgaagaagggggttgttcggcgctatagccggattcatagagaacggatgggagggactgatagacggatggtacgggttcagacaccaaaacgctcaaggcgaagggacagccgcagactataagagtacacaatccgctatcgatcaaattaccggtaagcttaatagactgatcgaaaaaactaatcaacaattcgaactaatcgataacgaatttacggaagtcgaaagacagattggcaatgtgataaattggactagagactctatgactgaggtttggtcatataacgccgaactgttagtcgcaatggaaaatcagcatacgatagaccttgccgatagcgaaatgaataagctatacgaaagggtgaaacgacaattgagggaaaacgccgaagaggacggaacagggtgtttcgaaatttttcacaaatgcgacgacgattgtatggctagtattaggaataatacatacgaccatagtaagtatagagaggaagcgatacagaataggattcaaatcgatcccgtaaaactgtctagcggatacaaagacgttatactgtggttctcattcggagcgtcatgtttcatactgcttgcaatcgctatggggttagtgttcatatgcgttaaaaacggaaatatgcgatgtactatttgtatttaa

SEQ ID NO: 35 - DNA, 1416 bp, Influenza A virus
  source 1..1416: mol_type = unassigned DNA
atgaatccaaatcagaaactatttgcattatctggagtggcaatagcacttagtgtactgaacttattgataggaatctcaaacgtcggattgaacgtatctctacatctaaaggaaaaaggacccaaacaggaggagaatttaacatgcacgaccattaatcaaaacaacactactgtagtagaaaacacatatgtaaataatacaacaataattaccaagggaactgatttgaaaacaccaagctatctgctgttgaacaagagcctgtgcaatgttgaagggtgggtcgtgatagcaaaagacaatgcagtaagatttggggaaagtgaacaaatcattgttaccagggagccatatgtatcatgcgacccaacaggatgcaaaatgtatgccttgcaccaagggactaccattaggaacaaacattcaaatggaacgattcatgacagaacagctttcagaggtctcatctccactccattgggcactccaccaaccgtaagtaacagtgactttatgtgtgttggatggtcaagcacaacttgccatgatgggattgctaggatgactatctgtatacaaggaaataatgacaatgctacagcaacggtttattacaacagaaggctgaccactaccattaagacctgggccagaaacattctgaggactcaagaatcagaatgtgtgtgccacaatggcacatgtgcagttgtaatgaccgacggatcggctagtagtcaagcctatacaaaagtaatgtatttccacaagggattagtagttaaggaggaggagttaaggggatcagccagacatattgaggaatgctcctgttatggacacaatcaaaaggtgacctgtgtgtgcagagataactggcagggagcaaacaggcctattatagaaattgatatgagcacattggagcacacaagtagatacgtgtgcactggaattctcacagacaccagcagacctggggacaaatctagtggtgattgttccaatccaataactgggagtcccggcgttccgggagtgaagggattcgggtttctaaatggggataacacatggcttggtaggaccatcagccccagatcaagaagtggattcgaaatgttgaaaatacctaatgcaggtactgatcccaattctagaatagcagaacgacaggaaattgtcgacaataacaattggtcaggctattccggaagctttattgactattggaatgataacagtgaatgctacaatccatgcttttacgtagagttaattagaggaagacccgaagaggctaaatacgtatggtgggcaagtaacagtctaattgccctatgtggaagcccattcccagttgggtctggttccttccccgatggggcacaaatccaatacttttcgtaa

SEQ ID NO: 36 - DNA, 1416 bp, unidentified
  misc_feature 1..1416: note = Deoptimized Influenza A virus
  source 1..1416: mol_type = other DNA
atgaatccgaaccaaaaattgttcgcattaagcggagtcgcaatcgcactaagcgtactgaatctgttgatagggataagtaacgtagggttgaacgtatcactacatttgaaagagaaagggcctaaacaggaagagaatttgacatgtactacaattaatcagaataatactaccgtagtcgaaaatacatacgttaacaatacaacaattattactaagggaaccgatctgaaaactccaagttatctgttactgaataaatctctatgtaacgttgagggatgggtagtgatcgcaaaggataacgccgttagattcggcgaaagcgaacagattatagtgactagagagccatacgtatcatgcgatccaaccggatgcaaaatgtacgcattacaccaagggacaactattaggaataaacactctaacggtacgatacacgatagaaccgcatttagggggttgattagtacaccactcggtacaccaccaaccgtttcgaatagcgactttatgtgcgtagggtggtctagtactacatgtcacgacggaatcgctagaatgacaatttgcatacaggggaataacgataacgctaccgcaaccgtatattataatagaagactaactactactattaagacatgggctaggaatatactgagaacgcaagaatccgaatgcgtttgtcataacggtacatgcgccgtagtgatgaccgacggatccgctagttcgcaagcatatactaaggtaatgtattttcacaaagggttagtagtgaaagaggaagagttgagggggtccgctagacatattgaggaatgctcatgttacggacataatcaaaaggtgacatgcgtatgtagagacaattggcaaggcgcaaatagacccattatcgaaatcgatatgagtacactcgaacatactagtagatatgtgtgtaccggaatactaaccgatacgagtagacccggcgataagtctagcggagattgctcaaacccaattaccggatcacccggagtgccaggcgttaagggattcggattccttaacggagacaatacatggttagggagaactattagtcctaggagtaggtccggattcgaaatgcttaagatacctaacgccggaaccgacccaaatagtaggattgccgaacgacaagagattgtcgacaataacaattggtccggatatagcggatcattcatagactattggaacgacaatagcgaatgctataacccatgtttttacgttgagttgattaggggtagacccgaagaggcaaaatacgtttggtgggcatctaacagtctaatcgcattatgcggatcaccatttcccgtaggtagcggatcatttcccgacggagcccaaattcaatattttagttaa

SEQ ID NO: 37 - DNA, 1683 bp, Influenza A virus
  source 1..1683: mol_type = unassigned DNA
atggaaacaatatcactaataactatactactagtagtaacagcaagcaatgcagataaaatctgcatcggccaccagtcaacaaactccacagaaactgtggacacgctaacagaaaccaatgttcctgtgacacatgccaaagaattgctccacacagagcataatggaatgctgtgtgcaacaagcctgggacatcccctcattctagacacatgcactattgaaggactagtctatggcaacccttcttgtgacctgctgttgggaggaagagaatggtcctacatcgtcgaaagatcatcagctgtaaatggaacgtgttaccctgggaatgtagaaaacctagaggaactcaggacactttttagttccgctagttcctaccaaagaatccaaatcttcccagacacaacctggaatgtgacttacactggaacaagcagagcatgttcaggttcattctacaggagtatgagatggctgactcaaaagagcggtttttaccctgttcaagacgcccaatacacaaataacaggggaaagagcattcttttcgtgtggggcatacatcacccacccacctataccgagcaaacaaatttgtacataagaaacgacacaacaacaagcgtgacaacagaagatttgaataggaccttcaaaccagtgatagggccaaggccccttgtcaatggtctgcagggaagaattgattattattggtcggtactaaaaccaggccaaacattgcgagtacgatccaatgggaatctaattgctccatggtatggacacgttctttcaggagggagccatggaagaatcctgaagactgatttaaaaggtggtaattgtgtagtgcaatgtcagactgaaaaaggtggcttaaacagtacattgccattccacaatatcagtaaatatgcatttggaacctgccccaaatatgtaagagttaatagtctcaaactggcagtcggtctgaggaacgtgcctgctagatcaagtagaggactatttggagccatagctggattcatagaaggaggttggccaggactagtcgctggctggtatggtttccagcattcaaatgatcaaggggttggtatggctgcagatagggattcaactcaaaaggcaattgataaaataacatccaaggtgaataatatagtcgacaagatgaacaagcaatatgaaataattgatcatgaattcagtgaggttgaaactagactcaatatgatcaataataagattgatgaccaaatacaagacgtatgggcatataatgcagaattgctagtactacttgaaaatcaaaaaacactcgatgagcatgatgcgaacgtgaacaatctatataacaaggtgaagagggcactgggctccaatgctatggaagatgggaaaggctgtttcgagctataccataaatgtgatgatcagtgcatggaaacaattcggaacgggacctataataggagaaagtatagagaggaatcaagactagaaaggcagaaaatagagggggttaagctggaatctgagggaacttacaaaatcctcaccatttattcgactgtcgcctcatctcttgtgcttgcaatggggtttgctgccttcctgttctgggccatgtccaatggatcttgcagatgcaacatttgtatataa

SEQ ID NO: 38 - DNA, 1683 bp, unidentified
  misc_feature 1..1683: note = Deoptimized Influenza A virus
  source 1..1683: mol_type = other DNA
atggagacaattagtctgattactatactattggtcgttacagcgtcaaacgctgacaaaatatgtataggccatcaatccactaattcaaccgaaacagtcgatacactaaccgaaacgaatgtgccagtgacacacgctaaagagctactgcataccgaacataacggaatgctatgcgctactagcctagggcatccactgatactcgatacatgtactatcgagggactcgtatacggtaatcctagttgcgatctactgttaggcggtagggaatggtcatacatagtcgaacgatcatccgccgtaaacggaacatgttatcccggtaatgtcgagaatctcgaagagcttaggacactattctcatccgctagctcataccaacgaatacagatttttcccgatactacatggaatgtgacatataccggaactagtagggcatgttccggatcattctatagatcaatgagatggttgacacaaaaatccggcttttaccctgtgcaagacgcacaatatacgaataataggggtaaatctatactattcgtatggggtatacatcatccacctacttataccgaacagactaatctgtatattagaaacgatacaactacatccgttacaaccgaagacttgaataggacattcaaacccgtaatcggacctagaccactagtgaacggattgcagggtagaatcgattactattggtccgtacttaagccagggcaaacacttagagtgagatctaacggtaatctaatcgcaccatggtacggacacgtacttagcggagggtcacacggtaggatacttaagaccgatctgaaaggggggaattgcgtagtgcaatgccaaaccgaaaaaggcggactgaattcgacactaccattccataatattagcaaatacgcattcggaacatgtcctaagtacgttagggtgaatagtctgaaactcgcagtgggattgagaaacgtacccgctagatcgagtagggggctattcggcgcaatcgcagggtttatcgaaggcggatggccaggactagttgccggatggtacggattccaacatagtaacgatcaaggcgtagggatggccgccgatagggatagcacacaaaaagcaatcgataagattactagtaaggttaataatatagtcgataagatgaataagcaatacgaaattatcgatcacgaatttagcgaagtcgaaactagactgaatatgataaataataagatagacgatcagatacaagacgtatgggcatataacgccgaactgttagtgttgcttgagaatcagaagacactcgacgaacacgacgcaaacgttaataatctgtataataaagtgaaaagagcactagggtctaacgctatggaggacggtaagggatgtttcgaactatatcataaatgcgacgatcaatgcatggagacaattagaaacggtacatataatcggagaaagtatagagaggaatctagactcgaaagacagaaaatcgaaggcgttaaactcgaatccgaaggaacatataagatactgactatttatagtacagtcgctagctcactagtgcttgctatgggattcgccgcattcttgttttgggctatgtcaaacggatcatgtaggtgtaatatttgtatttaa

SEQ ID NO: 39 - DNA, 1404 bp, Influenza A virus
  source 1..1404: mol_type = unassigned DNA
atgaatccaaatcaaaagataatagcacttggctctgtttctataactattgcgacaatatgtttactcatgcagattgccatcttagcaacgactatgacactacatttcaatgaatgtaccaacccatcgaacaatcaagcagtgccatgtgaaccaatcataatagaaaggaacataacagagatagtgcatttgaataatactaccatagagaaggaaagttgtcctaaagtagcagaatacaagaattggtcaaaaccgcaatgtcaaattacagggttcgcccctttctccaaggacaactcaattaggctttctgcaggcggggatatttgggtgacaagagaaccttatgtatcgtgcggtcttggtaaatgttaccaatttgcacttgggcagggaaccactttgaacaacaaacactcaaatggcacaatacatgataggagtccccatagaacccttttaatgaacgagttgggtgttccatttcatttgggaaccaaacaagtgtgcatagcatggtccagctcaagctgccatgatgggaaggcatggttacatgtttgtgtcactggggatgatagaaatgcgactgctagcatcatttatgatgggatgcttaccgacagtattggttcatggtctaagaacatcctcagaactcaggagtcagaatgcgtttgcatcaatggaacttgtacagtagtaatgactgatggaagtgcatcaggaagggctgatactaaaatactattcattagagaagggaaaattgtccacattggtccactgtcaggaagtgctcagcatgtggaggaatgctcctgttacccccggtatccagaagttagatgtgtttgcagagacaattggaagggctccaatagacccgtgctatatataaatgtggcagattatagtgttgattctagttatgtgtgctcaggacttgttggcgacacaccaagaaatgacgatagctccagcagcagtaactgcagggatcctaataacgagagagggggcccaggagtgaaagggtgggcctttgacaatggaaatgatgtttggatgggacgaacaatcaagaaagattcgcgctctggttatgagactttcagggtcgttggtggttggactacggctaattccaagtcacaaataaataggcaagtcatagttgacagtgataactggtctgggtattctggtatattctctgttgaaggaaaaacctgcatcaacaggtgtttttatgtggagttgataagagggagaccacaggagaccagagtatggtggacttcaaatagcatcattgtattttgtggaacttcaggtacctatggaacaggctcatggcctgatggagcgaatatcaatttcatgtctatataa

SEQ ID NO: 40 - DNA, 1404 bp, unidentified
  misc_feature 1..1404: note = Deoptimized Influenza A virus
  source 1..1404: mol_type = other DNA
atgaatccgaatcagaaaataatcgcattagggtccgtttcgattactatagcgactatatgcctattgatgcaaatcgcaatactcgcaacgactatgacattgcattttaacgaatgcactaatccctctaataatcaggccgttccatgcgaaccaatcataatcgaacggaatattaccgagatagtgcatcttaacaatacgactatcgaaaaagagtcatgccctaaggtagcggaatataaaaattggtctaagcctcaatgtcagattaccggattcgcaccattctctaaagataattcaattaggcttagcgcaggcggagatatatgggtgactagagagccatacgtaagttgcggactcggtaagtgttatcaattcgcattaggccaagggacaacccttaataataagcatagtaacggtactatacacgataggagtccacataggactcttcttatgaacgagttaggcgtaccattccatttagggactaaacaggtttgtatcgcatggtctagtagttcatgtcatgacggtaaggcatggttgcatgtttgcgttaccggcgacgatagaaacgctaccgcttcaatcatatacgacggtatgcttaccgattcaatcggatcatggtctaaaaatatacttagaacccaagagtccgaatgcgtatgtattaacggtacatgtacagtcgttatgacagacggatccgctagcggtagggccgatacaaagatactattcatacgcgaaggtaagatagtgcatatcggaccattgtccggatccgcacaacacgttgaggaatgctcatgttatcctagatatcccgaagtgagatgcgtatgtagagataattggaaagggtcaaatagacccgtactgtatataaacgttgccgattatagcgtcgatagttcatatgtgtgtagcggactagtgggcgatacacctagaaacgacgattcatctagtagttcgaattgtagggatcctaataacgaaagaggcggaccaggcgttaaagggtgggcattcgataacggtaacgacgtttggatggggagaactattaaaaaagattctagatcagggtatgagacattcagagtggtgggggggtggactaccgctaactctaagtctcaaattaatagacaggtgatagtcgatagcgataattggtcagggtattccggtatttttagcgttgagggtaagacatgtattaataggtgtttttatgtcgaattgattagggggcgaccacaagagactagggtttggtggactagtaattcgattatagtgttttgcggaactagcggaacatacggaaccggatcatggccagacggagcgaatataaattttatgtctatataa

SEQ ID NO: 41 - DNA, 2341 bp, Influenza virus
  source 1..2341: mol_type = unassigned DNA
  CDS 25..2298: note = SEQ ID NO: 42 is the translation of nucleotides 25-2298.
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggatgtaatggagtcaatgaaaaaagaagaaatggggatcacaactcattttcagagaaagagacgggtgagagacaatatgactaagaaaatgataacacagagaacaataggtaaaaagaagcagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgacaagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcatcgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagctccccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 42 - protein, 757 aa, Influenza virus
  REGION 1..757: note = translation of nucleotides 25-2298 of SEQ ID NO: 41
MDVNPTLLFLKVPAQNAISTTFPYTGDPPYSHGTGTGYTMDTVNRTHQYSEKGRWTTNTETGAPQLNPIDGPLPEDNEPSGYAQTDCVLEAMAFLEESHPGIFENSCIETMEVVQQTRVDKLTQGRQTYDWTLNRNQPAATALANTIEVFRSNGLTANESGRLIDFLKDVMESMKKEEMGITTHFQRKRRVRDNMTKKMITQRTIGKKKQRLNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYMTRNQPEWFRNVLSIAPIMFSNKMARLGKGYMFESKSMKLRTQIPAEMLASIDLKYFNDSTRKKIEKIRPLLIEGTASLSPGMMMGMFNMLSTVLGVSILNLGQKRYTKTTYWWDGLQSSDDFALIVNAPNHEGIQAGVDRFYRTCKLLGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFEIKKLWEQTRSKAGLLVSDGGPNLYNIRNLHIPEVCLKWELMDEDYQGRLCNPLNPFVSHKEIESMNNAVMMPAHGPAKNMEYDAVATTHSWIPKRNRSILNTSQRGVLEDEQMYQRCCNLFEKFFPSSSYRRPVGISSMVEAMVSRARIDARIDFESGRIKKEEFTEIMKICSTIEELRRQK

SEQ ID NO: 43 - DNA, 2341 bp, unidentified
  misc_feature 1..2341: note = synthetic
  source 1..2341: mol_type = other DNA
  CDS 25..2298: note = SEQ ID NO: 44 is the translation of nucleotides 25-2298.
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggacgttatggagtctatgaaaaaagaggaaatggggattacgacacattttcaacgaaaaagacgggttagggataatatgacaaaaaaaatgattacgcaacgaacaatcggaaagaaaaaacagagactgaataagcgatcatacttgattagggcacttacacttaacactatgactaaggacgccgaaaggggaaagctaaagcgtagagcaattgcaacacccggaatgcaaattagggggttcgtatacttcgtcgagacactcgctagatccatatgcgaaaagttagagcaatccggactgccagtcggggggaacgaaaaaaaagcgaaactcgctaacgtcgttagaaaaatgatgactaatagtcaggataccgaactgtcatttacgattaccggcgataatactaagtggaacgagaatcagaatcctagaatgtttctcgcaatgatcacatatatgacacgtaaccaacccgaatggtttagaaacgtactgtcaatcgcaccaattatgtttagcaataagatggctagattgggcaaggggtatatgtttgaatctaagagtatgaaattgcgaacacagatacctgccgaaatgctagcatcaatcgatctaaagtactttaacgatagtacacgaaaaaaaatcgaaaagattagaccgttactgatagagggaaccgccagcctatcccccggaatgatgatggggatgtttaatatgcttagtaccgtgttaggcgttagcatacttaacttagggcaaaaacgttatactaagactacatattggtgggacggactgcaatctagcgacgatttcgcactaatcgttaacgcacctaaccatgaggggatacaagccggagtcgatagattctatagaacatgcaaactgttagggattaatatgtctaaaaaaaagtcatacataaatagaaccggaacatttgaattcactagctttttttacagatacggattcgttgctaattttagtatggagttacctagtttcggagttagcggaattaacgaatccgccgatatgtcaatcggcgtaaccgttattaagaataatatgattaataacgatctagggccagcaaccgcacaaatggcattgcagttgttcataaaggattatcgttatacatatagatgtcatagaggcgatacacagatacagactagacgatcatttgaaatcaaaaaattgtgggagcaaactaggtctaaagccggactgttagtgtccgacggagggcctaatctatacaatattaggaatctgcatatacccgaagtgtgtctaaagtgggagcttatggacgaagactatcaggggagattgtgcaatccgcttaacccattcgttagccataaagagatagagtcaatgaataacgccgttatgatgccagcacacggacccgctaagaatatggaatacgacgcagtcgcaactacacatagttggataccgaaacggaatcgatccatactgaatacatcccaaagaggcgtactcgaagacgaacaaatgtaccaacggtgttgcaatctatttgaaaaattttttcctagtagtagctatagacgaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 44 - protein, 757 aa, unidentified; identical to SEQ ID NO: 42
  REGION 1..757: note = translation of nucleotides 25-2298 of SEQ ID NO: 43; note = Synthetic Construct

SEQ ID NO: 45 - DNA, 2341 bp, unidentified
  misc_feature 1..2341: note = synthetic
  source 1..2341: mol_type = other DNA
  CDS 25..2298: note = SEQ ID NO: 46 is the translation of nucleotides 25-2298.
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggacgttatggagtctatgaaaaaagaggaaatggggattacgacacattttcaacgaaaaagacgggttagggataatatgacaaaaaaaatgattacgcaacgaacaatcggaaagaaaaaacagagactgaataagcgatcatacttgattagggcacttacacttaacactatgactaaggacgccgaaaggggaaagctaaagcgtagagcaattgcaacacccggaatgcaaattagggggttcgtatacttcgtcgagacactcgctagatccatatgcgaaaagttagagcaatccggactgccagtcggggggaacgaaaaaaaagcgaaactcgctaacgtcgttagaaaaatgatgactaatagtcaggataccgaactgtcatttacgattaccggcgataatactaagtggaacgagaatcagaatcctagaatgtttctcgcaatgatcacatatatgacacgtaaccaacccgaatggtttagaaacgtactgtcaatcgcaccaattatgtttagcaataagatggctagattgggcaaggggtatatgtttgaatctaagagtatgaaattgcgaacacagatacctgccgaaatgctagcatcaatcgatctaaagtactttaacgatagtacacgaaaaaaaatcgaaaagattagaccgttactgatagagggaaccgccagcctatcccccggaatgatgatggggatgtttaatatgcttagtaccgtgttaggcgttagcatacttaacttagggcaaaaacgttatactaagactacatattggtgggacggactgcaatctagcgacgatttcgcactaatcgttaacgcacctaaccatgaggggatacaagccggagtcgatagattctatagaacatgcaaactgttagggattaatatgtctaaaaaaaagtcatacataaatagaaccggaacatttgaattcacaagttttttctatcgttatgggtttgttgccaatttcagcatggagctccccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 46 - protein, 757 aa, unidentified; identical to SEQ ID NO: 42
  REGION 1..757: note = translation of nucleotides 25-2298 of SEQ ID NO: 45; note = Synthetic Construct

SEQ ID NO: 47 - DNA, 2341 bp, Influenza virus
  source 1..2341: mol_type = unassigned DNA
agcgaaagcaggtcaattatattcaatatggaaagaataaaagaactaagaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgcaggatcagaccgagtgatggtatcacctctggctgtgacatggtggaataggaatggaccaataacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaggaatgatgatgttgatcaaagcttgattattgctgctaggaacatagtgagaagagctgcagtatcagcagatccactagcatctttattggagatgtgccacagcacacagattggtggaattaggatggtagacatccttaggcagaacccaacagaagagcaagccgtggatatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagtcagaggtgatctgaatttcgtcaatagggcgaatcagcgattgaatcctatgcatcaacttttaagacattttcagaaggatgcgaaagtgctttttcaaaattggggagttgaacctatcgacaatgtgatgggaatgattgggatattgccagacatgactccaagcatcgagatgtcaatgagaggagtgagaatcagcaaaatgggtgtagatgagtactccagcacggagagggtagtggtgagcattgaccgttttttgagaatccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactataacaaggccacgaagagactcacagttctcggaaaggatgctggcactttaactgaagacccagatgaaggcacagctggagtggagtccgctgttctgaggggattcctcattctgggcaaagaagacaagagatatgggccagcactaagcatcaatgaactgagcaaccttgcgaaaggagagaaggctaatgtgctaattgggcaaggagacgtggtgttggtaatgaaacggaaacgggactctagcatacttactgacagccagacagcgaccaaaagaattcggatggccatcaattagtgtcgaatagtttaaaaacgaccttgtttctact

SEQ ID NO: 48 - DNA, 2341 bp, unidentified
  misc_feature 1..2341: note = synthetic
  source 1..2341: mol_type = other DNA
agcgaaagcaggtcaattatattcaatatggagagaatcaaagagcttaggaatcttatgtcacaatctagaactagagagatactgactaagactacagtcgatcatatggctataatcaaaaaatatactagcggaagacaggaaaaaaatcccgcacttagaatgaaatggatgatggctatgaaataccctattacagccgataagcgaattaccgaaatgataccagagagaaacgaacagggacagacattgtggtctaaaatgaacgacgccggatccgatagagtgatggtttcgccactagccgtaacatggtggaatagaaacggacctattacgaatacagtgcattaccctaagatatacaaaacatatttcgaaagagtcgagagactgaaacacggaacattcggaccagtgcattttcggaatcaggttaagattagacgtagagtcgatattaatccagggcatgcagatctctccgctaaagaggcacaagacgttattatggaggtcgtgtttcctaacgaggtcggcgctaggatactgactagcgaatcgcaattgacaattacgaaagagaaaaaagaggaactccaggattgcaaaattagcccacttatggtcgcatatatgctcgaacgcgaattggttagaaagactagattcctaccagtcgcaggcggaacgtctagcgtgtatatcgaagtgttgcatctaacacagggaacatgttgggagcaaatgtatactccaggaggcgaagtgagaaacgacgacgttgatcaatcgctaatcatagccgctaggaatatagtgagaagggcagccgttagcgcagacccacttgcgtcactactcgaaatgtgccatagtacgcaaatcggagggattagaatggtcgatatccttaggcagaatcctacagaggaacaggccgtagacatatgcaaagccgcaatgggattgcgaattagctcatcattctcattcggagggtttacgtttaaacggactagcggatctagcgtaaaacgcgaagaggaagtgcttactggcaatctgcaaacactaaagattagggtgcatgagggatacgaagagtttacaatggtcggacgtagagcaaccgctatacttagaaaagcgactaggagactgatacaattgatcgttagcggaagggacgaacagtcaatcgccgaagcgataatagtcgcaatggtgttttcgcaagaggattgcatgattaaggccgttaggggggatctgaatttcgttaatagggctaatcagagactgaatcctatgcatcaattgcttagacattttcagaaagacgctaaagtgttgtttcagaattggggagtcgaacctatcgataacgttatgggtatgatagggatactgccagatatgacaccatcaatcgaaatgtcaatgagaggcgttaggattagtaagatgggcgtagacgaatactccagcactgagagagtggtagtgtcaatcgatagatttcttaggattagggatcagagaggcaacgtactgctatcacccgaagaagttagcgaaacacagggaaccgaaaaattgacaattacgtatagtagtagtatgatgtgggagattaacggaccagagtcagtgttagtgaatacatatcaatggataatacggaattgggagacagtgaaaatacaatggtcacagaatcctacaatgctatacaataagatggagttcgaaccttttcaatcgttagtgcctaaggccataagaggccaatatagtgggttcgttagaacattgtttcagcaaatgagagacgtactcggaacattcgataccgcacagataattaagctattgccattcgcagccgcaccacctaagcaatctagaatgcaattttctagctttaccgttaacgttaggggatccggaatgcgaatactcgttagggggaatagtccagtgtttaattacaataaggcaactaagagattgacagtgttaggcaaggacgcaggaacattgaccgaagacccagacgagggaaccgctggagtggaatccgcagtgcttagggggtttctgatactcggaaaggaggataagagatacggacctgcactatcgattaacgaactatctaatctcgctaaaggcgaaaaagcgaatgtgttaatcggacagggagacgtagtgttagtgatgaaacggaaacgcgatagctcaatactgacagactcacaaaccgctactaagagaattcggatggcaattaattagtgtcgaatagtttaaaaacgaccttgtttctact

SEQ ID NO: 49 - DNA, 2233 bp, Influenza virus
  source 1..2233: mol_type = unassigned DNA
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaggtcgctagcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggggtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaaccttgaacctgggacctttgatcttggggggctatatgaagcaattgaggagtgcctgattaatgatccctgggttttgcttaatgcttcttggttcaactccttccttacacatgcattgagttagttgtggcagtgctactatttgctatccatactgtccaaaaaagtaccttgtttctact

SEQ ID NO: 50 - DNA, 2233 bp, unidentified
  misc_feature 1..2233: note = synthetic
  source 1..2233: mol_type = other DNA
agcgaaagcaggtactgatccaaaatggaggatttcgttaggcaatgctttaatccaatgatagtcgagttagccgaaaagactatgaaagagtatggcgaagacctaaagattgagactaataaattcgccgcaatttgcacacaccttgaggtttgctttatgtattccgattttcactttattaacgaacagggagagtcaattatagtcgagttaggcgatccgaacgcattgctaaagcatagatttgaaattatagagggacgcgataggacaatggcatggaccgtagttaattcgatttgcaatacaaccggagccgaaaaaccgaaattcttacccgatctatacgattataaagagaataggtttatcgaaatcggagtgactagacgcgaagtgcatatttattatctcgaaaaagcgaataagattaagtccgaaaagacacacatacacatttttagctttaccggagaggaaatggcaacaaaagccgattatacacttgacgaagagtctagggctaggattaagactagactgtttacaattagacaggaaatggctagtagggggttgtgggatagctttagacaatccgaaagaggcgaagagacaatcgaagagagatttgaaattaccggaacaatgcgaaagcttgccgatcaatccctaccccccaatttctctagccttgagaattttagggcatacgttgacggattcgaacctaacggatatatagagggaaagctatcgcaaatgtctaaagaggttaacgctagaatcgaaccattcctaaagacaacacctagaccacttagactgccaaacggaccaccatgctcacagcgatctaagtttctgcttatggacgcactaaagttgtcaatcgaagacccatcacacgagggagaggggataccattgtacgacgcaattaagtgtatgcgaacatttttcggatggaaagagcctaacgtagtgaaaccacacgaaaaagggattaatccgaattatctgcttagttggaaacaggtgttagccgaattgcaggatatcgaaaacgaagagaaaattccgaaaactaagaatatgaaaaaaactagccaactgaaatgggcacttggcgagaatatggcacccgaaaaagtcgatttcgacgattgcaaagacgtcggcgatctaaagcaatacgatagcgacgaacccgaacttagatcactcgctagttggatacagaacgagttcaataaggcatgcgaattgaccgatagctcatggatagagcttgacgagataggcgaagacgtagcaccaatcgaacacatagcctctatgagacggaattattttacatccgaagtgtcacattgtagggcaacagagtatattatgaaaggggtgtatattaataccgcattgcttaacgctagttgcgccgcaatggacgatttccaactgataccgatgatctcgaagtgtagaacaaaagagggacgtagaaagactaatctgtatgggttcattattaagggaaggtctcatttaaggaacgatacagacgtagtgaatttcgttagtatggagtttagccttaccgatccgagactcgaaccacacaaatgggaaaagtattgcgtactagagataggggatatgttgattagatccgcaatcggacaggtttcgagaccaatgtttttgtacgttaggactaacggaacctcgaagattaaaatgaaatggggaatggagatgcgtagatgcctattgcaatcccttcagcaaatcgaatctatgatagaggccgaatctagcgttaaagagaaagatatgacaaaagagttttttgaaaataagtccgaaacatggccaatcggagagtcaccaaaaggggttgaggaatcctcaatcggaaaagtttgtagaacattgctcgcaaaatccgtattcaatagtctatacgccagcccacaactagagggattctctgctgagtcacgaaaactgttactgatagtgcaagcccttagggataatctcgaacccggaacattcgatctaggggggttgtacgaagcaatcgaagagtgtctgattaacgatccatgggtactgcttaacgctagttggtttaattcgttccttacacacgcactatcttagttgtggcagtgctactatttgctatccatactgtccaaaaaagtaccttgtttctact

SEQ ID NO: 51 - DNA, 1775 bp, Influenza virus
  source 1..1775: mol_type = unassigned DNA
  CDS 33..1730: note = SEQ ID NO: 52 is the translation of nucleotides 33-1730.
agcaaaagcaggggaaaataaaaacaaccaaaatgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatatctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacactccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 52 - protein, 565 aa, Influenza virus
  REGION 1..565: note = translation of nucleotides 33-1730 of SEQ ID NO: 51
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 53 - DNA, 1775 bp, unidentified
  misc_feature 1..1775: note = synthetic
  source 1..1775: mol_type = other DNA
  CDS 33..1730: note = SEQ ID NO: 54 is the translation of nucleotides 33-1730.
agcaaaagcaggggaaaataaaaacaaccaaaatgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgttagaggactcacataacggaaagctatgtaggcttaagggaatcgcaccactgcaattgggcaagtgtaatatagccggatggttgttggggaatcccgaatgcgatccactgttacccgttaggtcatggtcatatatagtcgagacacctaatagcgaaaacggaatttgttatcccggcgattttatcgattacgaagagcttagagagcaattgtctagcgttagttcattcgaaagattcgaaatttttccgaaagagtctagttggccaaatcataatactaacggagtgactgccgcatgctcacacgaaggcaagtctagcttttataggaatctgttgtggttgactgagaaagagggatcatatccgaaactgaaaaactcatacgtgaacaaaaagggaaaggaagtgttagtgttgtgggggatacaccatccaccaaatagtaaagagcaacagaatatatatcagaacgaaaacgcatacgttagcgtcgtaactagtaattataatagaaggtttacacccgaaatcgcagagagaccgaaagttagagaccaagccggaagaatgaattattattggacactactgaaacccggcgatacaattatattcgaagcgaacggaaatctgatcgcaccgatgtatgcattcgcactatctagggggttcggatccggaattattactagtaacgctagtatgcacgaatgtaacacgaagtgtcagactccactaggcgcaattaactctagtctgccatatcagaatatacatcccgtaacaatcggcgaatgcccaaaatacgttagatccgctaagcttagaatggttaccggactgagaaatacaccatcaatccaatctagggggttgttcggagcgatagccggatttatcgaaggggggtggacagggatgatagacggttggtacggatatcatcaccaaaacgaacagggatccggatacgcagccgatcagaaatcgacgcaaaacgctattaacggaattactaataaagtgaataccgtaatcgaaaaaatgaatatccaatttaccgcagtcggaaaggaattcaataagcttgagaaaagaatggagaatctgaataaaaaagtcgacgacggatttctagacatatggacttataacgccgaactgttagtgttgctcgaaaacgaaagaacactagactttcacgactcaaacgttaagaatctatacgaaaaagtgaaatcccaattgaaaaataacgctaaagagatagggaacggatgtttcgagttctatcataaatgcgataacgaatgtatggaatccgttaggaacggaacatacgattatcctaagtatagcgaagagtcaaaactgaatagggagaaagtcgacggagtgaaactcgaatcaatggggatatatcagatactggcaatctatagtacagtcgccagctcactggttcttttggtctccctgggggcaatcagtttctggatgtgttctaatggatctttgcagtgcagaatatgcatctgagattagaatttcagaaatatgaggaaaaacacccttgtttctact

SEQ ID NO: 54 - protein, 565 aa, unidentified; identical to SEQ ID NO: 52
  REGION 1..565: note = translation of nucleotides 33-1730 of SEQ ID NO: 53; note = Synthetic Construct

SEQ ID NO: 55 - DNA, 1565 bp, Influenza virus
  source 1..1565: mol_type = unassigned DNA
  CDS 46..1542: note = SEQ ID NO: 56 is the translation of nucleotides 46-1542.
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaatacctggaagaacatcccagtgcggggaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagagggaagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 56 - protein, 498 aa, Influenza virus
  REGION 1..498: note = translation of nucleotides 46-1542 of SEQ ID NO: 55
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 57 - DNA, 1565 bp, unidentified
  misc_feature 1..1565: note = synthetic
  source 1..1565: mol_type = other DNA
  CDS 46..1542: note = SEQ ID NO: 58 is the translation of nucleotides 46-1542.
agcaaaagcagggtagataatcactcactgagtgacatcaaaatcatggcgtcccaaggcaccaaacggtcttacgaacagatggagactgatggagaacgccagaatgccactgaaatcagagctagcgtcggaaaaatgatagggggaatcggaaggttttacatacaaatgtgtaccgaactcaaattgtccgattacgaagggagattgatccaaaatagtctgacaatcgaaagaatggtgttaagcgcattcgacgaaagacggaataagtatctcgaagagcatcctagcgcaggcaaggatccaaaaaaaaccggagggccaatctataggagagtgaacggaaagtggatgcgcgaactgatactgtacgataaagaggagattagacggatatggcgacaagcgaataacggagacgacgctactgccggactgacacatatgatgatatggcactctaatcttaacgacgctacataccaacggactagggcactcgttagaaccggaatggatcctagaatgtgctcacttatgcagggatctacactccctagacgatccggagccgcaggagcagccgttaagggagtcggaactatggttatggaactcgttagaatgataaaaagggggattaacgataggaatttttggagaggcgaaaacggacgtaaaactagaatcgcatacgaaagaatgtgcaatatactcaaagggaaattccaaaccgcagcgcaaaaagctatgatggatcaagttagggagtctaggaatccaggaaatgccgaattcgaagaccttacatttctcgctcggtccgcactaatccttcgcggatcagtcgcacacaaatcttgcttacccgcatgcgtatacggacctgcagtcgctagcggatacgatttcgaacgcgaagggtatagtctagtaggaattgatccatttagattgctccaaaattcgcaagtgtatagtctgattagacctaacgagaatcctgcacacaaatctcaactcgtatggatggcatgccatagtgccgcattcgaagaccttagagtgctatctttcataaagggaacgaaagtgttgcctaggggaaagctatctactaggggagtgcaaatcgctagtaacgagaatatggagactatggagtctagtacactcgaactgagatctagatattgggctattaggactagatccggagggaatacgaatcagcaacgagctagcgccgggcaaatctcaatccaacctacattttccgtgcaacggaatctgccattcgatcggacaacgattatggccgcattcaatgggaataccgagggacggactagcgatatgagaaccgaaattatcagaatgatggaatccgctagaccagaggacgtttcgtttcaaggacggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 58 - protein, 498 aa, unidentified; identical to SEQ ID NO: 56
  REGION 1..498: note = translation of nucleotides 46-1542 of SEQ ID NO: 57; note = Synthetic Construct

SEQ ID NO: 59 - DNA, 1413 bp, Influenza virus
  source 1..1413: mol_type = unassigned DNA
agcgaaagcaggggtttaaaatgaatccaaatcagaaaataacaaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaagcattcaaatgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaactggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgttaggcaagatgttgtggcaatgactgattggtcagggtatagcggaagtttcgttcaacatcctgagctaacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 60 - DNA, 1413 bp, unidentified
  misc_feature 1..1413: note = synthetic
  source 1..1413: mol_type = other DNA
agcgaaagcaggggtttaaaatgaatccaaatcagaaaataacaaccattggatcaatctgtctggtagtcggactaattagcctaatattgcaaatagggaatataatctcaatatggatttcgcattcaatccaaaccggatcacaaaatcatacaggcatatgcaatcagaatataattacttataaaaatagtacatgggtgaaagatactactagcgtgatactaaccggcaattctagtctatgtccgattagggggtgggctatatactctaaagacaatagtatacggatagggtctaagggagacgttttcgtaattagggaaccgtttataagttgttcacatctagagtgtaggaccttttttctgacacaaggcgcactattaaacgataagcattctaacggtacagttaaggataggtcaccttatagggcacttatgtcatgtcccgtaggcgaagcccctagtccatacaatagtagatttgaatccgttgcatggtccgctagcgcatgtcacgacggaatggggtggttgactatagggattagcggacccgataacggagccgttgccgtactgaaatataacggtataattaccgaaactattaagagttggcgtaaaaaaatattgcgtacacaagagtccgaatgcgcatgcgttaacggatcatgttttacaattatgactgacggacctagcgacgggttagcgtcatacaaaatttttaaaatcgaaaaaggcaaggttactaagtcaatcgagttaaacgcacctaattcgcattacgaagagtgttcatgttatcccgataccggaaaggttatgtgcgtttgtagggataattggcacggttcgaacagaccttgggtgtcattcgatcaaaatctagactatcaaatcggatatatatgtagcggagtgttcggcgataatcctagaccagaggacggtacaggcagctgtggaccggtttacgttgacggcgctaacggcgttaaggggtttagttatagatacggcaatggcgtatggatcggtaggactaagtcacatagttctagacacggatttgaaatgatatgggatcctaacggatggaccgaaaccgactcgaagtttagcgttaggcaagacgtagtcgctatgaccgattggtccgggtatagcggatcattcgtgcaacatccagagttaaccggattggattgtatgcgaccatgtttttgggttgagttgattagggggagaccgaaagagaaaactatatggactagcgcgagcagcatttctttttgtggcgtgaatagtgatactgtagattggtcttggccagacggtgctgagttgccattcagcattgacaagtagtctgttcaaaaaactccttgtttctact

SEQ ID NO: 61 - DNA, 1026 bp, Influenza virus
  source 1..1026: mol_type = unassigned DNA
agcgaaagcaggtagatattgaaagatgagtcttctaaccgaggtcgaaacgtacgtactctctatcatcccgtcaggccccctcaaagccgagatcgcacagagacttgaagatgtctttgcagggaagaacactgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcgctattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctac

SEQ ID NO: 62 - DNA, 890 bp, Influenza virus
  source 1..890: mol_type = unassigned DNA
agcaaaagcagggtgacaaagacataatggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcaccctcggtctggacatcgagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcagaatggaccaggcgatcatggataagaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcgggggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttaataataaaaaacacccttgtttctact

SEQ ID NO: 63 - DNA, 890 bp, unidentified
  misc_feature 1..890: note = synthetic
  source 1..890: mol_type = other DNA
agcaaaagcagggtgacaaagacataatggatccaaacactgtgtcaagctttcaggtagattgctttctttggcatgtccgcaaacgagttgcagaccaagaactaggtgatgccccattccttgaccgactgagacgggatcagaaatcccttaggggcaggggatcgaccctaggcctagacatcgaaaccgcaactagggccggaaagcagatcgtggagcgtatactgaaagaggagtccgacgaagcgcttaagatgactatggccagcgtacccgctagtcggtaccttaccgatatgacactcgaagagatgtcacgcgattggtctatgctaatccctaagcagaaagtggccggacctctatgtatacggatggaccaggcgattatggacaaaaacattatccttaaagcgaacttttccgtgatattcgatcgcctagagactctgatactgttgcgtgcattcacagaagagggagcaattgttggcgaaatttcaccattgccttctcttccaggacatactgctgaggatgtcaaaaatgcagttggagtcctcatcgggggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttaataataaaaaacacccttgtttctact

SEQ ID NO: 64 - protein, 757 aa, unidentified; identical to SEQ ID NO: 42

(19) United States
(12) Patent Application Publication
(10) Pub. No.: US 2024/0228978 A1
(43) Pub. Date: Jul. 11, 2024

(54) ATTENUATED INFLUENZA VIRUSES AND VACCINES

(71) Applicant: The Research Foundation for the State of University New York, Albany, NY (US)

(72) Inventors: Steffen Mueller, Kings Point, NY (US); Eckard Wimmer, East Setauket, NY (US); Bruce Futcher, Setauket, NY (US); Steve Skiena, Setauket, NY (US); Chen Yang, Shanghai (CN)

(73) Assignee: The Research Foundation for the State of University New York, Albany, NY (US)

(21) Appl. No.: 18/069,734

(22) Filed: Apr. 1, 2024

Related U.S. Application Data
(63) Continuation of application No. 16/436,475, filed on Jun. 10, 2019, now Pat. No. 11,549,101.
Application No. 16/436,475 is a continuation of application No. 14/777,204, filed on Sep. 15, 2015, now Pat. No. 10,316,294.
(60) Provisional application No. 61/794,617, filed on Mar. 15, 2013.

(51) Int. Cl. C12N 7/00; A61K 39/145

(52) U.S. Cl. CPC C12N 7/00; A61K 39/145; C12N 2760/16034; C12N 2760/16061; C12N 2760/16134; C12N 2760/16162

(57) ABSTRACT

This invention provides highly attenuated influenza viruses and vaccines. The attenuated viruses and vaccines proliferate well and have high safety factors. The attenuated viruses providing protective immunity from challenge by virus of the same subtype, as well as cross protection against heterologous viruses.

### CROSS-REFERENCE TO RELATED APPLICATIONS

This application is a continuation of U.S. application Ser. No. 16/436,475, filed Jun. 10, 2019, now U.S. Pat. No. 11,549,101, issued Jan. 10, 2023, which is a continuation of U.S. application Ser. No. 14/777,204, filed Sep. 15, 2015, which is the 371 of PCT/US2014/030027, filed Mar. 15, 2014, which claims the benefit of priority to U.S. Application No. 61/794,617, filed Mar. 15, 2013, all of which are incorporated herein by reference in their entireties.

### FEDERAL FUNDING

This invention was made with government support under AI015122 and AI075219 awarded the National Institutes of Health. The government has certain rights in the invention.

### REFERENCE TO AN ELECTRONIC SEQUENCE LISTING

This application contains a Sequence Listing which has been submitted electronically in xml format and is hereby incorporated by reference in its entirety. Said xml copy, created on Mar. 18, 2024, is named SeqList2-162152-49103.xml and is 169,032 bytes in size.

### FIELD OF THE INVENTION

This invention provides highly attenuated influenza viruses and vaccines. The attenuated viruses and vaccines proliferate well and have high safety factors. The attenuated viruses providing protective immunity from challenge by virus of the same subtype, as well as cross protection against heterologous viruses.

### BACKGROUND OF THE INVENTION

Influenza is a human disease that leads every year to >30,000 deaths in the US and several hundred thousand deaths globally (1). Major neutralization antigenic proteins, hemagglutinin (HA) and neuraminidase (NA) on the virion surface, provide protecting immunity, but undergo yearly genetic variation by point mutations (genetic drift). This renders the viruses resistant to population immunity and set the stage for seasonal epidemics. Further, influenza virus may acquire a new antigenic make-up (reassortment of heterologous genes, referred to as genetic shift) leading to pandemics. Because the flu is seasonal and variable, new vaccines must be produced every year. This is made more complex since more than one type or strain of influenza virus co-circulates in any flu season, a phenomenon demanding that more than one new vaccine may have to be developed every year.

Currently, only two major types of vaccines are licensed, the intramuscularly administered inactivated vaccines (“Flu shot”), and the live attenuated vaccine (LAIV), given intra-nasally (“FluMist®”). The efficacy of the two vaccines is suboptimal. The injectable inactivated vaccines that requires a large quantity of starting material (the equivalent of approximately 10¹⁰ plaque-forming units, PFU, per dose), are incapable of inducing significant cell-mediated immunity, which is being recognized as an important determinant of protection (4). Moreover, the overall efficacy of the inactivated vaccine in the U.S. adult population aged 18-65 years is only 59% (5). The LAIV “FluMist,” on the other hand, induces both humoral and cellular immunity but it is restricted in use to people 2 to 49 yr of age (6, 7). Moreover, recurrent administration of LAIV, which always uses the same attenuating viral backbone, could result in tolerance in repeat recipients (8).

Influenza viruses that have been classified as type A, B, and C, are enveloped, negative-strand RNA viruses of Orthomyxoviridae of which subtypes of type A are the major culprit of human disease (3). The viruses transcribe and replicate their multipartite genome in the cell nucleus, each segment encoding one or two polypeptides. Of these the most important antigenic molecules are the glycoproteins hemagglutinin (HA) and neuraminidase (NA).

### SUMMARY OF THE INVENTION

A long-held dogma posits that strong presentation to the immune system of the dominant influenza virus glycoprotein antigens hemagglutinin (HA) and neuraminidase (NA) is paramount for inducing protective immunity against influenza virus infection. It has now been discovered that attenuated viruses in which expression of the two dominant influenza virus glycoprotein antigens, HA and NA, is reduced, are highly effective in providing long lasting protective immunity against lethal wild type challenge and cross protection against diverse subtypes. Further, the viruses have exceptional safety profiles. Accordingly, the invention provides an attenuated influenza virus in which expression of hemagglutinin (HA) and neuraminidase (NA) is reduced. In certain embodiments, HA and NA are the only the only virus proteins having reduced expression. In other embodiments of the invention, the expression of one or more other virus proteins may also be reduced, such as, for example, PA, PB1, PB2, NP, NS, M1, or M2. In certain embodiments, when the expression of a virus proteins other than HA and NA is reduced, the reduction is small compared to the reduction of HA and NA. According to the invention, reduction in expression of virus proteins of the invention is accomplished by changes in protein encoding sequence, for example by lowering the codon pair bias of the protein-encoding sequence, substituting rare codons, modifying G+C content, modifying CG and/or TA (or UA) dinucleotide content, or combinations. Reduced expression can also be accomplished by modifications to the regulatory sequences of the proteins.

In one such embodiment, reducing the codon-pair bias comprises identifying a codon pair in the parent protein-encoding sequence having a codon-pair score that can be reduced, and reducing the codon-pair bias by substituting the codon pair with a codon pair that has a lower codon-pair score. In another such embodiment, reducing the codon-pair bias comprises rearranging the codons of a parent protein-encoding sequence. In certain embodiments, the reduced-expression HA protein-encoding sequence and the reduced-expression NA protein-encoding sequence individually have a codon pair bias less than −0.1, or less than −0.2, or less than −0.3, or less than −0.4. Codon pair bias of a protein-encoding sequence (i.e., an open reading frame) is calculated as described in Coleman et al., 2000 (ref. 12) and herein.

In an embodiment of the invention, expression of one or both of the HA protein-encoding sequence and the NA protein-encoding sequence is reduced by replacing one or more codons with synonymous codons that are less frequent in the host.

The invention further provides an influenza vaccine composition for inducing a protective immune response in a subject, wherein the vaccine composition comprises virus in which expression of HA is reduced and expression of NA is reduced. In certain embodiments, only expression of HA and NA is reduced. In some embodiments, expression of another virus protein is also reduced.

The invention also provides a method of eliciting a protective immune response in a subject comprising administering to the subject a prophylactically or therapeutically effective dose of a vaccine composition comprising an attenuated influenza virus, wherein expression of HA is reduced and expression of NA is reduced. In certain embodiments, only expression of HA and NA is reduced. In some embodiments, expression of another virus protein is also reduced. In an embodiment of the invention, an immune response is elicited that is effective against influenza of the same subtype as the attenuated virus of the vaccine. In another embodiment, an immune response is elicited that is effective against a heterologous influenza virus.

The invention also provides a method of making an attenuated influenza virus genome comprising a) obtaining the nucleotide sequence encoding the hemagglutinin protein of an influenza virus and the nucleotide sequence encoding the neuraminidase protein of an influenza virus, b) recoding the hemagglutinin-encoding nucleotide sequence to reduce expression and recoding the neuraminidase-encoding nucleotide sequence to reduce expression, and substituting the recoded nucleotide sequences into an influenza virus genome to make an attenuated influenza virus genome. In certain embodiments, only expression of HA and NA is reduced. In some embodiments, expression of another virus protein is also reduced.

### DESCRIPTION OF THE FIGURES

FIGS. 1A-1D. Construction of variants having reduced codon pair bias and phenotypes in tissue cultures. (FIG. 1A) NA^{Min} and HA^{Min} were designed (leaving 120-200 nt long wt sequences at 5′ and 3′ ends) and constructed by chemical synthesis. They were then used to replace by reverse genetics (13) one or two corresponding genes of wt PR8. The number of synonymous mutations is shown. (FIG. 1B) Recovered viruses were analyzed for plaque size phenotypes on MDCK monolayers. (FIG. 1C) Growth kinetics of wt PR8 and reduced codon-pair bias variants were analyzed on MDCK cells after infections at an MOI of 0.01. Every three hours post-infection, cell supernatants were collected and analyzed for virus titers by plaque assays. (FIG. 1D) Growth kinetics of wt PR8 and (NA+HA)^{Min} virus in A549 cells. Cells were infected at an MOI of 1.

FIGS. 2A-2B. Protein expression and mRNA levels in (NA+HA)^{Min}-infected in tissue culture cells. MDCK cells were infected with (NA+HA)^{Min} or wt PR8 at a MOI of 5. (FIG. 2A) Western blot analyses were performed to determine the viral protein expression the infected cells at 3 h and 6 h p.i. (FIG. 2B) Northern blot analyses were performed to determine mRNA levels of HA, NA, PB1 and GAPDH in (NA+HA)^{Min} or wt PR8-infected MDCK cells. At 3, 6, and 9 h p.i., cytoplasmic mRNA were collected and analyzed. For HA^{Min} and HA^{WT} transcript probes, the same 150 nt that recognized the common 3′ end of the respective genes was used. Similarly, the probes for NA^{Min} and NA^{WT} have the same 150 nt sequence corresponding to the common 3′ end of the NA genes.

FIGS. 3A-3F. Virus phenotypes in infected mice. (FIGS. 3A and 3B) Measurement of the median lethal dose (LD₅₀). Groups of five male Balb/C mice were intranasally infected with the (NA+HA)^{Min} variant at 10⁴, 10⁵, or 10⁶ PFU and the relative body weight and survival rate were monitored for 14 days p.i. Mice that lost 25% of their body weight were euthanized. LD₅₀ was calculated based on the method of Reed-Muench (24). (FIGS. 3C and 3D) Measurement of the median protective dose (PD₅₀). Groups of five male Balb/C mice were vaccinated with 10², 10¹, or 10⁰ PFU of (NA+HA)^{Min} on day 0. On day 28 post vaccination, all mice were challenged with 10⁵ PFU wt PR8 virus. The relative body weight and survival rate after challenge were monitored. PD₅₀ was calculated based on the method of Reed-Muench (24). (FIGS. 3E and 3F) Safe and effective vaccine range of the (NA+HA)^{Min} (open box) and wt PR8 virus (gray zone) were plotted. Any vaccine dose within this region warranted survival of the animals, and also completely protected them from lethal homogeneous challenge. Error bars represent SD.

FIGS. 4A-4B. Virus titers in lungs of infected mice. (FIG. 4A) Groups of three male Balb/C mice were infected with 10⁴ PFU of wt PR8 or (NA+HA)^{Min}. On day 1, 3, 5, 7, 9 and 11 p.i., the mice were euthanized and their lungs harvested and homogenized. Viral titers in the homogenates were determined by plaque assays on MDCK cells. * All wt PR8-infected mice were dead on day 5. #The virus titers in (NA+HA)^{Min}-infected mice after day 9 were undetectable (less than 4 PFU). (FIG. 4B) Comparison of virus titers in lungs of three mice each infected with wt PR8 or (NA+HA)^{Min} at a dose from 10¹ to 10⁴ PFU. The lungs of the animals were harvest on day 3, and plaque assays were performed to determine virus titers. Error bars represent SD.

FIGS. 5A-5D. Cross protection against H3N2 virus infections in (NA+HA)^{Min}(H1N1)-vaccinated mice. Groups of five Balb/c mice were vaccinated with (NA+HA)^{Min} at different doses. On day 28 post vaccination, mice were challenged with (FIGS. 5A and 5B) 100 LD₅₀ heterologous viruses A/Aichi/2/1968 (H3N2) virus (=1.5×10⁴ PFU). Survival rate and relative body weights were monitored for 14 days. All mice vaccinated with at least 10³ PFU of (NA+HA)^{Min} (H1N1) survived the lethal challenge. The cross protection PD₅₀ against H3N2 Aichi virus calculated is 237 PFU. (FIGS. 5C and 5D) Mice vaccinated with (NA+HA)^{Min} virus were also challenged with 100 LD₅₀ A/Victoria/3/75 (H3N2) virus (=3.2×10⁴ PFU). Survival rate and relative body weights were monitored for 14 days. All mice vaccinated with at least 10³ PFU of (NA+HA)^{Min} (H1N1) survived the lethal challenge. The cross protection PD₅₀ against H3N2 Victoria virus calculated is 147 PFU based on the method of Reed-Muench (24). Error bars represent SD.

FIG. 6. Hemagglutination inhibition (HAI) assay with serum of vaccinated mice. Mice were infected at different doses with PR8 or (NA+HA)^{Min}. Serum was collected on day 28 p.i. and antibody titers were determined by hemagglutination inhibition assays, as described in Material and Methods. Mice were then challenged with 10⁵ PFU wt PR8 and survival rates were monitored. Gray labeled dots indicated mice that did not survive.

FIGS. 7A-7D. LD₅₀ and PD₅₀ values of NA^{Min} in mice. (FIGS. 7A and 7B) Groups of five male Balb/c mice were infected intranasally with different doses of NA^{Min} variant. The relative body weight and survival rate were monitored for 14 days. The LD₅₀ calculated was 2.4×10⁵ PFU. (C and D) Groups of five males were vaccinated with different dose of NA^{Min} variant, 28 days p.i., mice were challenged with 10⁵ PFU wt influenza A/PR/8/34 (PR8). The relative body weight and survival rate were monitored for 14 days. Error bars represent SD.

FIGS. 8A-8B. Long term protection of (NA+HA)^{Min}-vaccinated mice. Groups of five Balb/c mice (5-6 weeks) were infected intranasally with (NA+HA)^{Min} at different doses. After seven months, mice were challenged with 10⁵ PFU wt PR8. Their body weight and survival rate were monitored for 14 days. Error bars represent SD.

FIGS. 9A-9D. Composition of (NA+HA)^{Min} virus. WT and (NA+HA)^{Min} virus were purified by sucrose gradient. Equivalent amounts of PFUs were compared to determine the relative amounts of the indicated virus proteins. (FIG. 9A) Commassie stain. (FIGS. 9B and 9C) silver stain. (FIG. 9D) Western blot.

FIGS. 10A-10B. Expression of virus proteins and mRNAs in MDCK cells infected with WT influenza or (NA+HA)^{Min}. (FIG. 10A)³⁵S labeled proteins in infected MDCK cells. (FIG. 10B) Northern analysis of viral mRNAs expressed in infected MDCK.

FIG. 11. Passive immunization with Serum from PR8-(NA+HA)^{Min} vaccinated mice protects naïve mice from homologous WT PR8 challenge. FIG. 11 shows mice passively immunized with PR8-(NA+HA)^{Min} sera survived and remained healthy upon challenge with WT virus.

FIGS. 12A-12B. Passive immunization with serum from PR8-(NA+HA)^{Min} (H1N1) vaccinated mice protects naïve mice from heterologous challenge with an H3N2 virus. FIG. 11 shows mice passively immunized with PR8-(NA+HA)^{Min} sera maintained weight (Panel A) and had improved survival (Panel B) when challenged with H3N2 virus.

FIGS. 13A-13B. Assessment of cross protection against H3N2 viruses conferred by immunization with PR8-(NA+HA)^{Min}. (FIG. 13A) Inhibition of hemagglutination by sera from PR8-(NA+HA)^{Min} immunized mice. (FIG. 13B) Neutralization of virus infection of MDCK cells by sera from PR8-(NA+HA)^{Min} immunized mice.

FIG. 14. Growth of WT and PR8-(NA+HA)^{Min} virus in MDCK cells and MDCK cells transfected to express α-2,6-sialyltransferase.

FIG. 15. T cell responses in lungs of Balb/C mice 7 days post-infection. Cell numbers are expressed as total cell count in lung (left panels) or percentage of CD45⁺ cells (right panels).

FIG. 16. B cell responses in lungs of Balb/C mice 7 days post-infection. Cell numbers are expressed as total cell count in lung (upper panel) or percentage of CD45⁺ cells (lower panels).

FIG. 17. T cell responses in spleens of Balb/C mice 7 days post-infection.

FIG. 18. T cell responses in spleens of Balb/C mice 7 days post-infection.

FIGS. 19A-19I. Immune cell infiltration of lung tissue 3 days post-infection. (FIG. 19A) CD45⁺ leukocytes, (FIG. 19B) CD45⁺ Ly6G^{high} polymorphonuclear leukocytes (PMN), (FIG. 19C) CD45⁺ CD11c⁺ I-A^{d+} F4/80⁻ dendritic cells, (FIG. 19D) CD45⁺ NKp46⁺ natural killer cells, (FIG. 19E) CD45⁺ CD11b⁺ Ly6C^{high}Ly6G⁻ inflammatory monocytes, (FIG. 19F) CD45⁺ I-A^{d+} F4/80⁺ macrophages, (G) CD45⁺ CD3⁺ CD4⁺ T helper cells, (FIG. 19H) CD45⁺ CD19⁺ B cells, and (FIG. 19I) CD45⁺ CD19⁺ IgM⁺ B cells.

### DETAILED DESCRIPTION

The present invention relates to the production of attenuated influenza viruses that can be used to protect against viral infection and disease. A basic premise in flu vaccination is adequate delivery of HA and NA to vaccine recipients assuming that a very high dose (“Flu shot”) or a dose corresponding to live viral infection (“FluMist”) of these traditionally dominant antigenic polypeptides alone are sufficient for adequate vaccine efficacy. Those expectations aside, the present invention benefits from a contrary approach. The invention provides attenuated influenza viruses in which expression of HA and NA is reduced, which have excellent growth properties useful to vaccine production, yet possess an extraordinary safety profile and enhanced protective characteristics. The attenuated viruses proliferate nearly as well as wild type virus, have highly attenuated phenotypes, as revealed by LD₅₀ values, are unusually effective in providing protective immunity against challenge by influenza virus of the same subtype, and also provide protective immunity against challenge by influenza virus of other subtypes.

In certain attenuated viruses of the invention, the expression of one or more other virus proteins may also be reduced, such as, for example, PA, PB1, PB2, NP, NS, M1, or M2. In certain embodiments, when the expression of a virus proteins other than HA and NA is reduced, the reduction is small compared to the reduction of HA and NA.

In certain attenuated influenza viruses of the invention, expression of hemagglutinin (HA) and neuraminidase (NA) is reduced, and expression of other influenza proteins (i.e., NP, M (including M1 and M2), NS, PA, PB1, and PB2 protein is not substantially changed (i.e., substantially reduced or increased). In an embodiment of the invention, expression of NP, PA, PB1, and PB2 is not substantially reduced. That expression of the NP, M (including M1 and M2), NS, PA, PB1, and PB2 protein encoding sequences is not substantially reduced means that in embodiments where there is a small change in expression of one or more of those proteins (e.g., NP, PA, PB1, PB2, M, and or M), the change in expression of those proteins has little or no effect on attenuation. Little or no effect on attenuation includes one or both of the following: 1) Any reduced expression of NP, M (including M1 and M2), NS, PA, PB1, or PB2 does not reduce viral replication or viral infectivity more than 20% when the NP, M (including M1 and M2), NS, PA, PB1, or PB2 is expressed at the reduced level in a test influenza virus in which only the level of that protein is reduced; 2) The level of expression of NP, M (including M1 and M2), NS, PA, PB1, or PB2 is reduced by less than 20% in the attenuated virus in which expression of HA and NA is reduced.

In certain embodiments of the invention, the attenuated influenza viruses of the invention comprise a recoded hemagglutinin (HA) nucleic acid and a recoded neuraminidase (NA) nucleic acid. In certain of these embodiments, another virus protein, such as NP, M (including M1 and M2), NS, PA, PB1, or PB2, is recoded. In others of these embodiments, other protein encoding sequences (i.e., NP, M (including M1 and M2), NS, PA, PB1, and PB2 protein encoding sequences are not recoded. That the NP, M (including M1 and M2), NS, PA, PB1, and PB2 protein encoding sequences are not recoded does not exclude mutations and other variations in those sequences, but only means that any mutations or variations made in those sequences have little or no effect on attenuation. Little or no effect on attenuation includes one or both of the following: 1) The mutations or variations in the NP, M (including M1 and M2), NS, PA, PB1, or PB2 sequence do not reduce viral replication or viral infectivity more than 20% when the variant NP, M (including M1 and M2), NS, PA, PB1, or PB2 nucleic acid sequence is the only variant in a test influenza virus; 2) Mutations or variations in any of the NP, M (including M1 and M2), NS, PA, PB1, or PB2 nucleic acid represent fewer than 10% of the nucleotides in that coding sequence.

The viruses of the invention are highly attenuated. In embodiments of the invention, compared to wild type, the viruses are at least 5,000 fold attenuated, or at least 10,000 fold attenuated, or at least 20,000 fold attenuated, or at least 33,000 fold attenuated, or at least 50,000 fold attenuated, of at least 100,000 fold attenuated in the BALB/c mouse model compared to a wild type virus having proteins of the same sequence but encoded by a different nucleotide sequence.

The attenuated viruses are also highly protective against wild type virus of the same subtype. In embodiments of the invention, the protective dose (PD₅₀) of the viruses is less than 100 PFU, or less than 50 PFU, or less than 20 PFU, or less than 10 PFU, or less than 5 PFU, when measured by a mouse model, such as exemplified herein.

The attenuated viruses of the invention also exhibit a large margin of safety (i.e., the difference between LD₅₀ and PD₅₀), thus have high safety factors, defined herein as the ratio of LD₅₀/PD₅₀. In certain embodiments of the invention, the safety factor is at least 10², or at least 10³, or at least 10⁴, or at least 10⁵, or at least 2×10⁵, or at least 5×10⁵, or at least 10⁶, or at least 2×10⁶, or at least 5×10⁶. In certain embodiments, the safety factor is from 10² to 10³, or from 10³ to 10⁴, or from 10⁴ to 10⁵, or from 10⁵ to 10⁶.

The attenuated viruses of the invention are also highly protective against heterologous viruses. In certain embodiments of the invention, the protective dose (PD₅₀) of an attenuated virus of the invention is less than 1000 PFU, or less than 500 PFU, or less than 200 PFU, or less than 100 PFU, when measured by a mouse model, such as exemplified herein

The recoding of HA and NA protein encoding sequences of the attenuated viruses of the invention can have been made utilizing any algorithm or procedure known in the art or newly devised for recoding a protein encoding sequence. According to the invention, nucleotide substitutions are engineered in multiple locations in the HA and NA coding sequences, wherein the substitutions introduce a plurality of synonymous codons into the genome. In certain embodiments, the synonymous codon substitutions alter codon bias, codon pair bias, the density of infrequent codons or infrequently occurring codon pairs, RNA secondary structure, CG and/or TA (or UA) dinucleotide content, C+G content, translation frameshift sites, translation pause sites, the presence or absence microRNA recognition sequences or any combination thereof, in the genome. The codon substitutions may be engineered in multiple locations distributed throughout the HA and NA coding sequences, or in the multiple locations restricted to a portion of the HA and NA coding sequences. Because of the large number of defects (i.e., nucleotide substitutions) involved, the invention provides a means of producing stably attenuated viruses and live vaccines.

As discussed further below, in some embodiments, a virus coding sequence is recoded by substituting one or more codon with synonymous codons used less frequently in the influenza host (e.g., humans, birds, pigs). In some embodiments, a virus coding sequence is recoded by substituting one or more codons with synonymous codons used less frequently in the influenza virus. In certain embodiments, the number of codons substituted with synonymous codons is at least 5. In some embodiments, at least 10, or at least 20 codons are substituted with synonymous codons.

In some embodiments, virus codon pairs are recoded to reduce (i.e., lower the value of) codon-pair bias. In certain embodiments, codon-pair bias is reduced by identifying a codon pair in an HA or NA coding sequence having a codon-pair score that can be reduced and reducing the codon-pair bias by substituting the codon pair with a codon pair that has a lower codon-pair score. In some embodiments, this substitution of codon pairs takes the form of rearranging existing codons of a sequence. In some such embodiments, a subset of codon pairs is substituted by rearranging a subset of synonymous codons. In other embodiments, codon pairs are substituted by maximizing the number of rearranged synonymous codons. It is noted that while rearrangement of codons leads to codon-pair bias that is reduced (made more negative) for the virus coding sequence overall, and the rearrangement results in a decreased CPS at many locations, there may accompanying CPS increases at other locations, but on average, the codon pair scores, and thus the CPB of the modified sequence, is reduced. In some embodiments, recoding of codons or codon-pairs can take into account altering the G+C content of the HA and NA coding sequences. In some embodiments, recoding of codons or codon-pairs can take into account altering the frequency of CG and/or TA dinucleotides in the HA and NA coding sequences.

In certain embodiments, the recoded (i.e., reduced-expression) HA protein-encoding sequence has a codon pair bias less than −0.1, or less than −0.2, or less than −0.3, or less than −0.4. In certain embodiments, the recoded (i.e., reduced-expression) NA protein-encoding sequence has a codon pair bias less than −0.1, or less than −0.2, or less than −0.3, or less than −0.4. In certain embodiments, the codon pair bias of the recoded HA protein encoding sequence is reduced by at least 0.1, or at least 0.2, or at least 0.3, or at least 0.4, compared to the parent HA protein encoding sequence from which it is derived. In certain embodiments, the codon pair bias of the recoded NA protein encoding sequence is reduced by at least 0.1, or at least 0.2, or at least 0.3, or at least 0.4, compared to the parent NA protein encoding sequence from which it is derived. In certain embodiments, rearrangement of synonymous codons of the HA protein-encoding sequence provides a codon-pair bias reduction of at least 0.1, or at least 0.2, or at least 0.3, or at least 0.4, parent HA protein encoding sequence from which it is derived. In certain embodiments, rearrangement of synonymous codons of the NA protein-encoding sequence provides a codon-pair bias reduction of at least 0.1, or at least 0.2, or at least 0.3, or at least 0.4, parent NA protein encoding sequence from which it is derived.

Usually, these substitutions and alterations are made and reduce expression of the encoded virus proteins without altering the amino acid sequence of the encoded protein. In certain embodiments, the invention also includes alterations in the HA and/or NA coding sequences that result in substitution of non-synonymous codons an amino acid substitutions in the encoded protein, which may or may not be conservative.

Most amino acids are encoded by more than one codon. See the genetic code in Table 1. For instance, alanine is encoded by GCU, GCC, GCA, and GCG. Three amino acids (Leu, Ser, and Arg) are encoded by six different codons, while only Trp and Met have unique codons. “Synonymous” codons are codons that encode the same amino acid. Thus, for example, CUU, CUC, CUA, CUG, UUA, and UUG are synonymous codons that code for Leu. Synonymous codons are not used with equal frequency. In general, the most frequently used codons in a particular organism are those for which the cognate tRNA is abundant, and the use of these codons enhances the rate and/or accuracy of protein translation. Conversely, tRNAs for the rarely used codons are found at relatively low levels, and the use of rare codons is thought to reduce translation rate and/or accuracy.

TABLE 1
Genetic Code^{a}

| | U | C | A | G | |
|---|---|---|---|---|---|
| U | Phe | Ser | Tyr | Cys | U |
| | Phe | Ser | Tyr | Cys | C |
| | Leu | Ser | STOP | STOP | A |
| | Leu | Ser | STOP | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

^{a}The first nucleotide in each codon encoding a particular amino acid is shown in the left-most column; the second nucleotide is shown in the top row; and the third nucleotide is shown in the right-most column.

### Codon Bias

As used herein, a “rare” codon is one of at least two synonymous codons encoding a particular amino acid that is present in an mRNA at a significantly lower frequency than the most frequently used codon for that amino acid. Thus, the rare codon may be present at about a 2-fold lower frequency than the most frequently used codon. Preferably, the rare codon is present at least a 3-fold, more preferably at least a 5-fold, lower frequency than the most frequently used codon for the amino acid. Conversely, a “frequent” codon is one of at least two synonymous codons encoding a particular amino acid that is present in an mRNA at a significantly higher frequency than the least frequently used codon for that amino acid. The frequent codon may be present at about a 2-fold, preferably at least a 3-fold, more preferably at least a 5-fold, higher frequency than the least frequently used codon for the amino acid. For example, human genes use the leucine codon CTG 40% of the time, but use the synonymous CTA only 7% of the time (see Table 2). Thus, CTG is a frequent codon, whereas CTA is a rare codon. Roughly consistent with these frequencies of usage, there are 6 copies in the genome for the gene for the tRNA recognizing CTG, whereas there are only 2 copies of the gene for the tRNA recognizing CTA. Similarly, human genes use the frequent codons TCT and TCC for serine or and 22/of/the time, respectively, but the rare codon TCG only 50 of the time. TCT and TCC are read, via wobble, by the same tRNA, which has 10 copies of its gene in the genome, while TCG is read by a tRNA with only 4 copies. It is well known that those mRNAs that are very actively translated are strongly biased to use only the most frequent codons. This includes genes for ribosomal proteins and glycolytic enzymes. On the other hand, mRNAs for relatively non-abundant proteins may use the rare codons.

TABLE 2
Codon usage in Homo sapiens (source:
http://www.kazusa.or.jp/codon/)

| Amino Acid | Codon | Number | /1000 | Fraction |
|---|---|---|---|---|
| Gly | GGG | 636457.00 | 16.45 | 0.25 |
| Gly | GGA | 637120.00 | 16.47 | 0.25 |
| Gly | GGT | 416131.00 | 10.76 | 0.16 |
| Gly | GGC | 862557.00 | 22.29 | 0.34 |
| Glu | GAG | 1532589.00 | 39.61 | 0.58 |
| Glu | GAA | 1116000.00 | 28.84 | 0.42 |
| Asp | GAT | 842504.00 | 21.78 | 0.46 |
| Asp | GAC | 973377.00 | 25.16 | 0.54 |
| Val | GTG | 1091853.00 | 28.22 | 0.46 |
| Val | GTA | 273515.00 | 7.07 | 0.12 |
| Val | GTT | 426252.00 | 11.02 | 0.18 |
| Val | GTC | 562086.00 | 14.53 | 0.24 |
| Ala | GCG | 286975.00 | 7.42 | 0.11 |
| Ala | GCA | 614754.00 | 15.89 | 0.23 |
| Ala | GCT | 715079.00 | 18.48 | 0.27 |
| Ala | GCC | 1079491.00 | 27.90 | 0.40 |
| Arg | AGG | 461676.00 | 11.93 | 0.21 |
| Arg | AGA | 466435.00 | 12.06 | 0.21 |
| Ser | AGT | 469641.00 | 12.14 | 0.15 |
| Ser | AGC | 753597.00 | 19.48 | 0.24 |
| Lys | AAG | 1236148.00 | 31.95 | 0.57 |
| Lys | AAA | 940312.00 | 24.30 | 0.43 |
| Asn | AAT | 653566.00 | 16.89 | 0.47 |
| Asn | AAC | 739007.00 | 19.10 | 0.53 |
| Met | ATG | 853648.00 | 22.06 | 1.00 |
| Ile | ATA | 288118.00 | 7.45 | 0.17 |
| Ile | ATT | 615699.00 | 15.91 | 0.36 |
| Ile | ATC | 808306.00 | 20.89 | 0.47 |
| Thr | ACG | 234532.00 | 6.06 | 0.11 |
| Thr | ACA | 580580.00 | 15.01 | 0.28 |
| Thr | ACT | 506277.00 | 13.09 | 0.25 |
| Thr | ACC | 732313.00 | 18.93 | 0.36 |
| Trp | TGG | 510256.00 | 13.19 | 1.00 |
| End | TGA | 59528.00 | 1.54 | 0.47 |
| Cys | TGT | 407020.00 | 10.52 | 0.45 |
| Cys | TGC | 487907.00 | 12.61 | 0.55 |
| End | TAG | 30104.00 | 0.78 | 0.24 |
| End | TAA | 38222.00 | 0.99 | 0.30 |
| Tyr | TAT | 470083.00 | 12.15 | 0.44 |
| Tyr | TAC | 592163.00 | 15.30 | 0.56 |
| Leu | TTG | 498920.00 | 12.89 | 0.13 |
| Leu | TTA | 294684.00 | 7.62 | 0.08 |
| Phe | TTT | 676381.00 | 17.48 | 0.46 |
| Phe | TTC | 789374.00 | 20.40 | 0.54 |
| Ser | TCG | 171428.00 | 4.43 | 0.05 |
| Ser | TCA | 471469.00 | 12.19 | 0.15 |
| Ser | TCT | 585967.00 | 15.14 | 0.19 |
| Ser | TCC | 684663.00 | 17.70 | 0.22 |
| Arg | CGG | 443753.00 | 11.47 | 0.20 |
| Arg | CGA | 239573.00 | 6.19 | 0.11 |
| Arg | CGT | 176691.00 | 4.57 | 0.08 |
| Arg | CGC | 405748.00 | 10.49 | 0.18 |
| Gln | CAG | 1323614.00 | 34.21 | 0.74 |
| Gln | CAA | 473648.00 | 12.24 | 0.26 |
| His | CAT | 419726.00 | 10.85 | 0.42 |
| His | CAC | 583620.00 | 15.08 | 0.58 |
| Leu | CTG | 1539118.00 | 39.78 | 0.40 |
| Leu | CTA | 276799.00 | 7.15 | 0.07 |
| Leu | CTT | 508151.00 | 13.13 | 0.13 |
| Leu | CTC | 759527.00 | 19.63 | 0.20 |
| Pro | CCG | 268884.00 | 6.95 | 0.11 |
| Pro | CCA | 653281.00 | 16.88 | 0.28 |
| Pro | CCT | 676401.00 | 17.48 | 0.29 |
| Pro | CCC | 767793.00 | 19.84 | 0.32 |

The propensity for highly expressed genes to use frequent codons is called “codon bias.” A gene for a ribosomal protein might use only the 20 to 25 most frequent of the 61 codons, and have a high codon bias (a codon bias close to 1), while a poorly expressed gene might use all 61 codons, and have little or no codon bias (a codon bias close to 0). It is thought that the frequently used codons are codons where larger amounts of the cognate tRNA are expressed, and that use of these codons allows translation to proceed more rapidly, or more accurately, or both. The PV capsid protein, for example, is very actively translated, and has a high codon bias.

### Codon Pair Bias

In addition, a given organism has a preference for the nearest codon neighbor of a given codon A, referred to a bias in codon pair utilization. A change of codon pair bias, without changing the existing codons, can influence the rate of protein synthesis and production of a protein.

Codon pair bias may be illustrated by considering the amino acid pair Ala-Glu, which can be encoded by 8 different codon pairs. If no factors other than the frequency of each individual codon (as shown in Table 2) are responsible for the frequency of the codon pair, the expected frequency of each of the 8 encodings can be calculated by multiplying the frequencies of the two relevant codons. For example, by this calculation the codon pair GCA-GAA would be expected to occur at a frequency of 0.097 out of all Ala-Glu coding pairs (0.23×0.42; based on the frequencies in Table 2). In order to relate the expected (hypothetical) frequency of each codon pair to the actually observed frequency in the human genome the Consensus CDS (CCDS) database of consistently annotated human coding regions, containing a total of 14,795 human genes, was used. This set of genes is the most comprehensive representation of human coding sequences. Using this set of genes the frequencies of codon usage were re-calculated by dividing the number of occurrences of a codon by the number of all synonymous codons coding for the same amino acid. As expected the frequencies correlated closely with previously published ones such as the ones given in Table 2. Slight frequency variations are possibly due to an oversampling effect in the data provided by the codon usage database at Kazusa DNA Research Institute (http://www.kazusa.or.jp/codon/codon.html) where 84949 human coding sequences were included in the calculation (far more than the actual number of human genes). The codon frequencies thus calculated were then used to calculate the expected codon-pair frequencies by first multiplying the frequencies of the two relevant codons with each other (see Table 3 expected frequency), and then multiplying this result with the observed frequency (in the entire CCDS data set) with which the amino acid pair encoded by the codon pair in question occurs. In the example of codon pair GCA-GAA, this second calculation gives an expected frequency of 0.098 (compared to 0.097 in the first calculation using the Kazusa dataset). Finally, the actual codon pair frequencies as observed in a set of 14,795 human genes was determined by counting the total number of occurrences of each codon pair in the set and dividing it by the number of all synonymous coding pairs in the set coding for the same amino acid pair (Table 3; observed frequency). Frequency and observed/expected values for the complete set of 3721 (61²) codon pairs, based on the set of 14,795 human genes, are provided herewith as Supplemental Table 1.

TABLE 3
Codon Pair Scores Exemplified by the Amino Acid Pair Ala-Glu

| amino | | expected | observed | |
|---|---|---|---|---|
| acid pair | codon pair | frequency | frequency | obs/exp ratio |
| AE | GCAGAA | 0.098 | 0.163 | 1.65 |
| AE | GCAGAG | 0.132 | 0.198 | 1.51 |
| AE | GCCGAA | 0.171 | 0.031 | 0.18 |
| AE | GCCGAG | 0.229 | 0.142 | 0.62 |
| AE | GCGGAA | 0.046 | 0.027 | 0.57 |
| AE | GCGGAG | 0.062 | 0.089 | 1.44 |
| AE | GCTGAA | 0.112 | 0.145 | 1.29 |
| AE | GCTGAG | 0.150 | 0.206 | 1.37 |
| Total | | 1.000 | 1.000 | |

If the ratio of observed frequency/expected frequency of the codon pair is greater than one the codon pair is said to be overrepresented. If the ratio is smaller than one, it is said to be underrepresented. In the example the codon pair GCA-GAA is overrepresented 1.65 fold while the coding pair GCC-GAA is more than 5-fold underrepresented.

Many other codon pairs show very strong bias; some pairs are under-represented, while other pairs are over-represented. For instance, the codon pairs GCCGAA (AlaGlu) and GATCTG (AspLeu) are three- to six-fold under-represented (the preferred pairs being GCAGAG and GACCTG, respectively), while the codon pairs GCCAAG (AlaLys) and AATGAA (AsnGlu) are about two-fold over-represented. It is noteworthy that codon pair bias has nothing to do with the frequency of pairs of amino acids, nor with the frequency of individual codons. For instance, the under-represented pair GATCTG (AspLeu) happens to use the most frequent Leu codon, (CTG).

As discussed more fully below, codon pair bias takes into account the score for each codon pair in a coding sequence averaged over the entire length of the coding sequence. According to the invention, codon pair bias is determined by

$$C P B = {\sum }_{i = 1}^{k} \frac{CPSi}{k - 1} .$$

Accordingly, similar codon pair bias for a coding sequence can be obtained, for example, by minimized codon pair scores over a subsequence or moderately diminished codon pair scores over the full length of the coding sequence.

### Calculation of Codon Pair Bias.

Every individual codon pair of the possible 3721 non-“STOP” containing codon pairs (e.g., GTT-GCT) carries an assigned “codon pair score,” or “CPS” that is specific for a given “training set” of genes. The CPS of a given codon pair is defined as the log ratio of the observed number of occurances over the number that would have been expected in this set of genes (in this example the human genome). Determining the actual number of occurrences of a particular codon pair (or in other words the likelyhood of a particular amino acid pair being encoded by a particular codon pair) is simply a matter of counting the actual number of occurances of a codon pair in a particular set of coding sequences. Determining the expected number, however, requires additional calculations. The expected number is calculated so as to be independent of both amino acid frequency and codon bias similarly to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in the human genome.

To perform these calculations within the human context, the most recent Consensus CDS (CCDS) database of consistently annotated human coding regions, containing a total of 14,795 genes, was used. This data set provided codon and codon pair, and thus amino acid and amino-acid pair frequencies on a genomic scale.

The paradigm of Federov et al. (2002), was used to further enhanced the approach of Gutman and Hatfield (1989). This allowed calculation of the expected frequency of a given codon pair independent of codon frequency and non-random associations of neighboring codons encoding a particular amino acid pair.

$$S ( {P}_{ij} ) = ln ( \frac{{N}_{O} ( {P}_{ij} )}{{N}_{E} ( {P}_{ij} )} ) = ln ( \frac{{N}_{O} ( {P}_{ij} )}{F ( {C}_{i} ) F ( {C}_{j} ) {N}_{O} ( {X}_{ij} )} )$$

In the calculation, Pᵢⱼ is a codon pair occurring with a frequency of No(Pᵢⱼ) in its synonymous group. Cᵢ and C_{f} are the two codons comprising Pᵢⱼ, occurring with frequencies F(Cᵢ) and F(Cⱼ) in their synonymous groups respectively. More explicitly, F(Cᵢ) is the frequency that corresponding amino acid Xᵢ is coded by codon Cᵢ throughout all coding regions and F(Cᵢ)=No(Cᵢ) No(Xᵢ), where No(Cᵢ) and No(Xᵢ) are the observed number of occurrences of codon Cᵢ and amino acid Xᵢ respectively. F(Cⱼ) is calculated accordingly. Further, No(Xᵢⱼ) is the number of occurrences of amino acid pair Xᵢⱼ throughout all coding regions. The codon pair bias score S(Pᵢⱼ) of Pᵢⱼ was calculated as the log-odds ratio of the observed frequency Nₒ(Pᵢⱼ) over the expected number of occurrences of Nₑ(Pᵢⱼ).

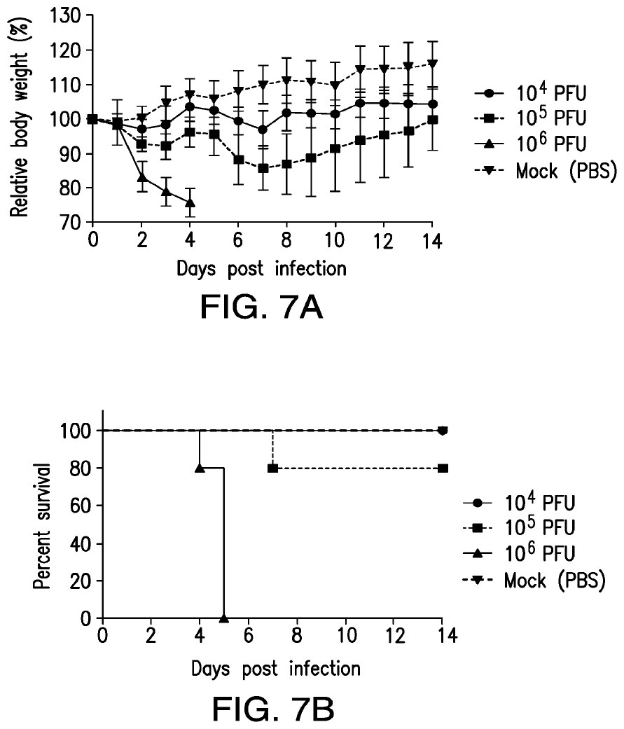

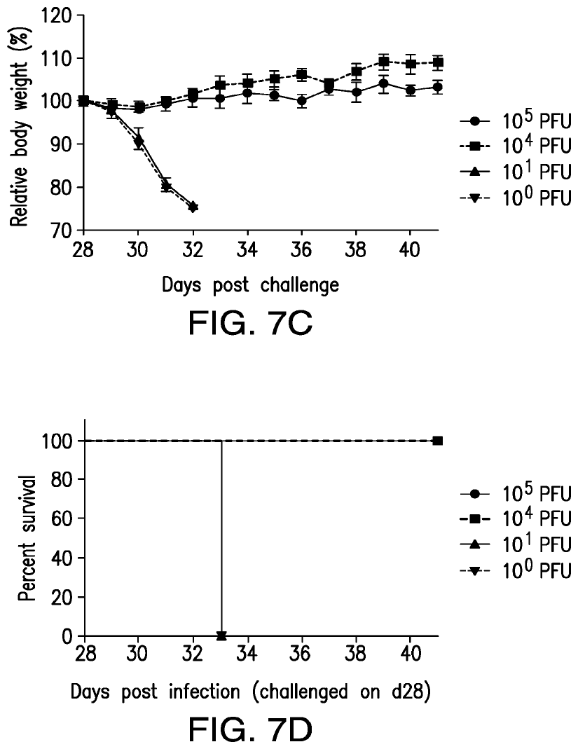

Using the formula above, it was then determined whether individual codon pairs in individual coding sequences are over- or under-represented when compared to the corresponding genomic Nₑ(Pᵢⱼ) values that were calculated by using the entire human CCDS data set. This calculation resulted in positive S(Pᵢⱼ) score values for over-represented and negative values for under-represented codon pairs in the human coding regions (FIG. 7).

The “combined” codon pair bias of an individual coding sequence was calculated by averaging all codon pair scores according to the following formula:

$$S ( {P}_{ij} ) = {\sum }_{l = 1}^{k} \frac{S ( {P}_{ij} ) l}{k - 1} .$$

The codon pair bias of an entire coding region is thus calculated by adding all of the individual codon pair scores comprising the region and dividing this sum by the length of the coding sequence.

Calculation of codon pair bias, implementation of algorithm to alter codon-pair bias.

An algorithm was developed to quantify codon pair bias. Every possible individual codon pair was given a “codon pair score”, or “CPS”. CPS is defined as the natural log of the ratio of the observed over the expected number of occurrences of each codon pair over all human coding regions, where humans represent the host species of the instant vaccine virus to be recoded.

$$C P S = ln [ \frac{F ( AB ) o}{\frac{F ( A ) \times F ( B )}{F ( X ) \times F ( Y )} \times F ( X Y )} )$$

Although the calculation of the observed occurrences of a particular codon pair is straightforward (the actual count within the gene set), the expected number of occurrences of a codon pair requires additional calculation. We calculate this expected number to be independent both of amino acid frequency and of codon bias, similar to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in the human genome

Using these calculated CPSs, any coding region can then be rated as using over- or under-represented codon pairs by taking the average of the codon pair scores, thus giving a Codon Pair Bias (CPB) for the entire gene.

$$CPB = {\sum }_{i = 1}^{k} \frac{CPSi}{k - 1}$$

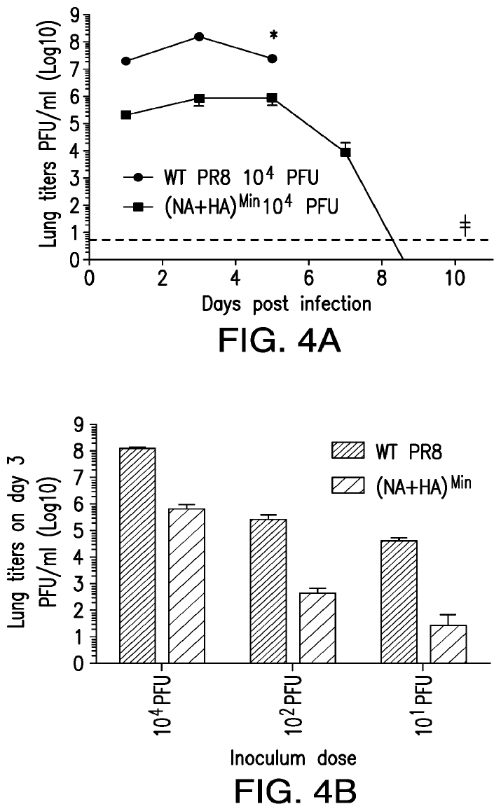

The CPB has been calculated for all annotated human genes using the equations shown and plotted (FIG. 4). Each point in the graph corresponds to the CPB of a single human gene. The peak of the distribution has a positive codon pair bias of 0.07, which is the mean score for all annotated human genes. Also there are very few genes with a negative codon pair bias. Equations established to define and calculate CPB were then used to manipulate this bias.

Algorithm for reducing codon-pair bias.

Recoding of protein-encoding sequences may be performed with or without the aid of a computer, using, for example, a gradient descent, or simulated annealing, or other minimization routine. An example of the procedure that rearranges codons present in a starting sequence can be represented by the following steps:
1) Obtain wildtype viral genome sequence.
2) Select protein coding sequences to target for attenuated design.
3) Lock down known or conjectured DNA segments with non-coding functions.
4) Select desired codon distribution for remaining amino acids in redesigned proteins.
5) Perform random shuffle of at least two synonymous unlocked codon positions and calculate codon-pair score.
6) Further reduce (or increase) codon-pair score optionally employing a simulated annealing procedure.
7) Inspect resulting design for excessive secondary structure and unwanted restriction site:
   if yes->go to step (5) or correct the design by replacing problematic regions with wildtype sequences and go to step (8).
8. Synthesize DNA sequence corresponding to virus design.
9. Create viral construct and assess viral phenotype:
   if too attenuated, prepare subclone construct and goto 9;
   if insufficiently attenuated, goto 2.

Attenuation of viruses by reducing codon pair bias is disclosed in WO 2008/121992 and WO 2011/044561, which are incorporated by reference.

### Attenuated Influenza Viruses

According to the invention, viral attenuation is accomplished by reducing expression of HA and NA coding sequences. One way to reduce expression of the coding sequences is by a reduction in codon pair bias, but other methods can also be used, alone or in combination. While codon bias may be changed, adjusting codon pair bias is particularly advantageous. For example, attenuating a virus through codon bias generally requires elimination of common codons, and so the complexity of the nucleotide sequence is reduced. In contrast, codon pair bias reduction or minimization can be accomplished while maintaining far greater sequence diversity, and consequently greater control over nucleic acid secondary structure, annealing temperature, and other physical and biochemical properties.

Codon pair bias of a protein-encoding sequence (i.e., an open reading frame) is calculated as set forth above and described in Coleman et al., 2000 (ref. 12).

Viral attenuation and induction or protective immune responses can be confirmed in ways that are well known to one of ordinary skill in the art, including but not limited to, the methods and assays disclosed herein. Non-limiting examples include plaque assays, growth measurements, reduced lethality in test animals, and protection against subsequent infection with a wild type virus.

In preferred embodiments, the invention provides viruses that are highly attenuated, and induce immunity against a plurality of influenza types and/or subtypes. Such flu varieties include viruses bearing all possible HA-NA combinations. Currently, there are 16 recognized hemagglutinins and nine neuraminidases, each of which has mutational variants. Examples of type A subtypes include, but are not limited to, H10N7, H10N1, H10N2, H10N3, H10N4, H10N5, H10N6, H10N7, H10N8, H10N9, H11N1, H 11N2, H11N3, H11N4, H11N6, H11N8, H11N9, H12N1, H12N2, H12N4, H12N5, H12N6, H12N8, H12N9, H13N2, H13N3, H13N6, H13N9, H14N5, H14N6, H15N2, H15N8, H15N9, H16N3, H1N1, H1N2, H1N3, H1N5, H1N6, H1N8, H1N9, H2N1, H2N2, H2N3, H2N4, H2N5, H2N6, H2N7, H2N8, H2N9, H3N1, H3N2, H3N3, H3N4, H3N5, H3N6, H3N8, H3N9, H4N1, H4N2, H4N3, H4N4, H4N5, H4N6, H4N7, H4N8, H4N9, H5N1, H5N2, H5N3, H5N4, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N7, H7N8, H7N9, H8N2, H8N4, H8N5, H9N1, H9N2, H9N3, H9N4, H9N5, H9N6, H9N7, H9N8, H9N9. Some subtypes of interest include, but are not limited to, H1N1 (one variant of which caused Spanish flu in 1918, another of which is pandemic in 2009), H2N2 (a variant of which caused Asian Flu in 1957), H3N2 (a variant of which caused Hong Kong Flu in 1968, H5N1 (a current pandemic threat), H7N7 (which has unusual zoonotic potential), and H1N2 (endemic in humans and pigs). Examples of attenuated influenza protein coding sequences are provided below.

TABLE 4
Reduced-Expression Influenza A Virus Genes

| | WT Coding Sequence | | | Recoded Coding Sequence | | |
|---|---|---|---|---|---|---|
| | SEQ ID | | | SEQ ID | Recoded | |
| Gene | NO: | CDS | CPB | NO | Codons | CPB |

H10N7 (A/northern shoveler/California/HKWF392sm/2007)(Avian)

| | | | | | | |
|---|---|---|---|---|---|---|
| HA | 1 | 1-1683 | 0.018 | 2 | 1-561 | −0.441 |
| NA | 3 | 1-1494 | 0.009 | 4 | 1-498 | −0.449 |

H1N1 (A/New York/3568/2009)(Human)

| | | | | | | |
|---|---|---|---|---|---|---|
| HA | 5 | 1-1698 | 0.043 | 6 | 1-566 | −0.410 |
| NA | 7 | 1-1407 | 0.005 | 8 | 1-469 | −0.456 |

H1N2 (A/New York/211/2003)(Human)

| | | | | | | |
|---|---|---|---|---|---|---|
| HA | 9 | 1-1695 | 0.036 | 10 | 1-565 | −0.421 |
| NA | 11 | 1-1407 | 0.034 | 12 | 1-469 | −0.476 |

H2N2 (A/Albany/22/1957)(Human)

| | | | | | | |
|---|---|---|---|---|---|---|
| HA | 13 | 1-1686 | 0.040 | 14 | 1-562 | −0.422 |
| NA | 15 | 1-1407 | 0.008 | 16 | 1-469 | −0.453 |

H3N2 (A/New York/933/2006)(Human)

| | | | | | | |
|---|---|---|---|---|---|---|
| HA | 17 | 1-1698 | 0.027 | 18 | 1-566 | −0.447 |
| NA | 19 | 1-1407 | 0.041 | 20 | 1-469 | −0.463 |

H5N1 (A/Jiangsu/1/2007)(Human)

| | | | | | | |
|---|---|---|---|---|---|---|
| HA | 21 | 1-1701 | 0.017 | 22 | 1-567 | −0.435 |
| NA | 23 | 1-1347 | 0.009 | 24 | 1-449 | −0.407 |

H7N2 (A/chicken/NJ/294508-12/2004)(Avian)

| | | | | | | |
|---|---|---|---|---|---|---|
| HA | 25 | 1-1656 | 0.036 | 26 | 1-552 | −0.377 |
| NA | 27 | 1-1359 | 0.013 | 28 | 1-453 | −0.491 |

H7N3 (A/Canada/rv504/2004)(Human)

| | | | | | | |
|---|---|---|---|---|---|---|
| HA | 29 | 1-1701 | 0.029 | 30 | 1-567 | −0.405 |
| NA | 31 | 1-1407 | 0.042 | 32 | 1-469 | −0.413 |

H7N7 (A/Netherlands/219/03)(Human)

| | | | | | | |
|---|---|---|---|---|---|---|
| HA | 33 | 1-1707 | 0.008 | 34 | 1-569 | −0.447 |
| NA | 35 | 1-1413 | −0.009 | 36 | 1-471 | −0.423 |

H9N2 (A/Hong Kong/1073/99)(Human)

| | | | | | | |
|---|---|---|---|---|---|---|
| HA | 37 | 1-1680 | 0.021 | 38 | 1-560 | −0.440 |
| NA | 39 | 1-1401 | 0.020 | 40 | 1-467 | −0.453 |

### Vaccine Compositions

The present invention provides a vaccine composition for inducing a protective immune response in a subject comprising any of the attenuated viruses described herein and a pharmaceutically acceptable carrier.

It should be understood that an attenuated virus of the invention, where used to elicit a protective immune response in a subject or to prevent a subject from becoming afflicted with a virus-associated disease, is administered to the subject in the form of a composition additionally comprising a pharmaceutically acceptable carrier. Pharmaceutically acceptable carriers are well known to those skilled in the art and include, but are not limited to, one or more of 0.01-0. IM and preferably 0.05M phosphate buffer, phosphate-buffered saline (PBS), or 0.900 saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate and magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Pharmaceutically acceptable carriers can further comprise minor amounts of auxiliary substances such as wetting or emulsifying agents, preservatives and other additives, such as, for example, antimicrobials, antioxidants and chelating agents, which enhance the shelf life and/or effectiveness of the active ingredients. The instant compositions can, as is well known in the art, be formulated so as to provide quick, sustained or delayed release of the active ingredient after administration to a subject.

In various embodiments of the instant vaccine composition, the attenuated virus (i) does not substantially alter the synthesis and processing of viral proteins in an infected cell; (ii) produces similar amounts of virions per infected cell as wt virus; and/or (iii) exhibits substantially lower virion-specific infectivity than wt virus. In further embodiments, the attenuated virus induces a substantially similar immune response in a host animal as the corresponding wt virus.

This invention also provides a modified host cell line specially isolated or engineered to be permissive for an attenuated virus that is inviable in a wild type host cell. Since the attenuated virus cannot grow in normal (wild type) host cells, it is absolutely dependent on the specific helper cell line for growth. This provides a very high level of safety for the generation of virus for vaccine production. Various embodiments of the instant modified cell line permit the growth of an attenuated virus, wherein the genome of said cell line has been altered to increase the number of genes encoding rare tRNAs.

In addition, the present invention provides a method for eliciting a protective immune response in a subject comprising administering to the subject a prophylactically or therapeutically effective dose of any of the vaccine compositions described herein. This invention also provides a method for preventing a subject from becoming afflicted with a virus-associated disease comprising administering to the subject a prophylactically effective dose of any of the instant vaccine compositions. In embodiments of the above methods, the subject has been exposed to a pathogenic virus. “Exposed” to a pathogenic virus means contact with the virus such that infection could result.

The invention further provides a method for delaying the onset, or slowing the rate of progression, of a virus-associated disease in a virus-infected subject comprising administering to the subject a therapeutically effective dose of any of the instant vaccine compositions.

As used herein, “administering” means delivering using any of the various methods and delivery systems known to those skilled in the art. Administering can be performed, for example, intranasally, intraperitoneally, intracerebrally, intravenously, orally, transmucosally, subcutaneously, transdermally, intradermally, intramuscularly, topically, parenterally, via implant, intrathecally, intralymphatically, intralesionally, pericardially, or epidurally. An agent or composition may also be administered in an aerosol, such as for pulmonary and/or intranasal delivery. Administering may be performed, for example, once, a plurality of times, and/or over one or more extended periods.

Eliciting a protective immune response in a subject can be accomplished, for example, by administering a primary dose of a vaccine to a subject, followed after a suitable period of time by one or more subsequent administrations of the vaccine. A suitable period of time between administrations of the vaccine may readily be determined by one skilled in the art, and is usually on the order of several weeks to months. The present invention is not limited, however, to any particular method, route or frequency of administration.

A “subject” means any animal or artificially modified animal. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems, and CD155tg transgenic mice expressing the human poliovirus receptor CD155. In a preferred embodiment, the subject is a human. Preferred embodiments of birds are domesticated poultry species, including, but not limited to, chickens, turkeys, ducks, and geese.

A “prophylactically effective dose” is any amount of a vaccine that, when administered to a subject prone to viral infection or prone to affliction with a virus-associated disorder, induces in the subject an immune response that protects the subject from becoming infected by the virus or afflicted with the disorder. “Protecting” the subject means either reducing the likelihood of the subject's becoming infected with the virus, or lessening the likelihood of the disorder's onset in the subject, by at least two-fold, preferably at least ten-fold. For example, if a subject has a 1% chance of becoming infected with a virus, a two-fold reduction in the likelihood of the subject becoming infected with the virus would result in the subject having a 0.5% chance of becoming infected with the virus. Most preferably, a “prophylactically effective dose” induces in the subject an immune response that completely prevents the subject from becoming infected by the virus or prevents the onset of the disorder in the subject entirely.

As used herein, a “therapeutically effective dose” is any amount of a vaccine that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

Certain embodiments of any of the instant immunization and therapeutic methods further comprise administering to the subject at least one adjuvant. An “adjuvant” shall mean any agent suitable for enhancing the immunogenicity of an antigen and boosting an immune response in a subject. Numerous adjuvants, including particulate adjuvants, suitable for use with both protein- and nucleic acid-based vaccines, and methods of combining adjuvants with antigens, are well known to those skilled in the art. Suitable adjuvants for nucleic acid based vaccines include, but are not limited to, Quil A, imiquimod, resiquimod, and interleukin-12 delivered in purified protein or nucleic acid form. Adjuvants suitable for use with protein immunization include, but are not limited to, alum, Freund's incomplete adjuvant (FIA), saponin, Quil A, and QS-21.

The invention also provides a kit for immunization of a subject with an attenuated virus of the invention. The kit comprises the attenuated virus, a pharmaceutically acceptable carrier, an applicator, and an instructional material for the use thereof. In further embodiments, the attenuated virus may be one or more poliovirus, one or more rhinovirus, one or more influenza virus, etc. More than one virus may be preferred where it is desirable to immunize a host against a number of different isolates of a particular virus. The invention includes other embodiments of kits that are known to those skilled in the art. The instructions can provide any information that is useful for directing the administration of the attenuated viruses.

Throughout this application, various publications, reference texts, textbooks, technical manuals, patents, and patent applications have been referred to. The teachings and disclosures of these publications, patents, patent applications and other documents in their entireties are hereby incorporated by reference into this application to more fully describe the state of the art to which the present invention pertains. However, the citation of a reference herein should not be construed as an acknowledgement that such reference is prior art to the present invention.

It is to be understood and expected that variations in the principles of invention herein disclosed can be made by one skilled in the art and it is intended that such modifications are to be included within the scope of the present invention. The following Examples further illustrate the invention, but should not be construed to limit the scope of the invention in any way. Detailed descriptions of conventional methods, such as those employed in the construction of recombinant plasmids, transfection of host cells with viral constructs, polymerase chain reaction (PCR), and immunological techniques can be obtained from numerous publications, including Sambrook et al. (1989) and Coligan et al. (1994). All references mentioned herein are incorporated in their entirety by reference into this application. The contents of WO 2008/121992 and WO 2011/044561 are incorporated by reference.

### EXAMPLES

### Example 1—Construction and Characterization of an HA and NA Codon Pair-Bias Reduced Influenza Virus in Tissue Culture

To achieve attenuation of influenza virus PR8, codon pair bias was reduced (introducing underrepresented codon pairs) in viral genes HA and NA according to computer algorithms (12, 13) and chemical synthesis (14), in order to reduce the expression level of the targeted viral genes.

Cells and viruses. MDCK, A549 and HEK293 T cell lines were maintained in DMEM supplemented with 10% FBS at 37° C. Influenza A/PR/8/34 (PR8) was cultured in MDCK cells.

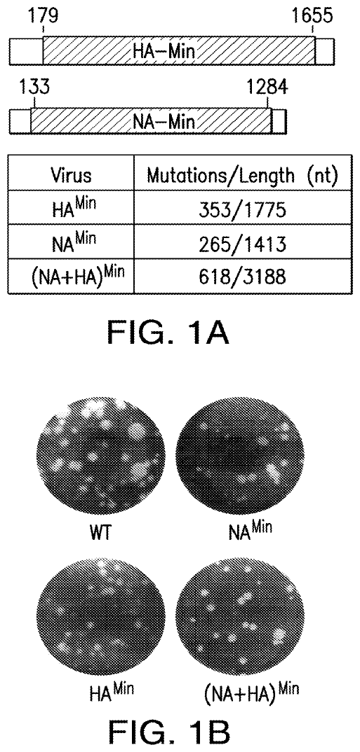

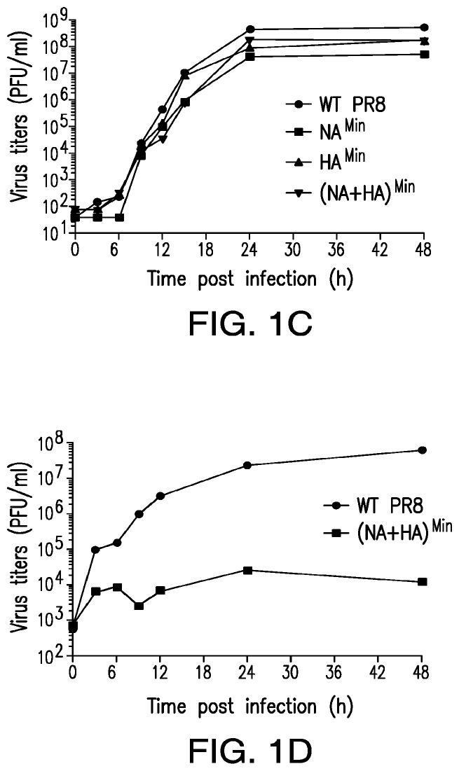

Variant (NA+HA)^{Min} (618/3188 nt changes), combining the HA^{Min} (SEQ ID NO:53) and NA^{Min} (SEQ ID NO:60) genes, expressed growth and plaque phenotypes in MDCK cells comparable to those of the individual HA^{Min} and NA^{Min} variants (FIG. 1B, C). Similarly, a variant with a codon-pair bias reduced NA gene (NA^{Min}, 265/1413 synonymous mutations; FIG. 1A) also replicated well in MDCK cells (FIG. 1C) and expressed an only slightly smaller plaque size phenotype (FIG. 1B) than wt PR8. In A549 cells the (NA+HA)^{Min} variant was highly attenuated (FIG. 1D), growing to a final titer three to four orders of magnitudes lower than wt PR8. A549 cells retain a complex signaling network that is related to the innate host response (15, 16).

### Example 2—Levels of NA mRNA and HA Protein are Reduced in (NA+HA)^{Min}-Infected Cells

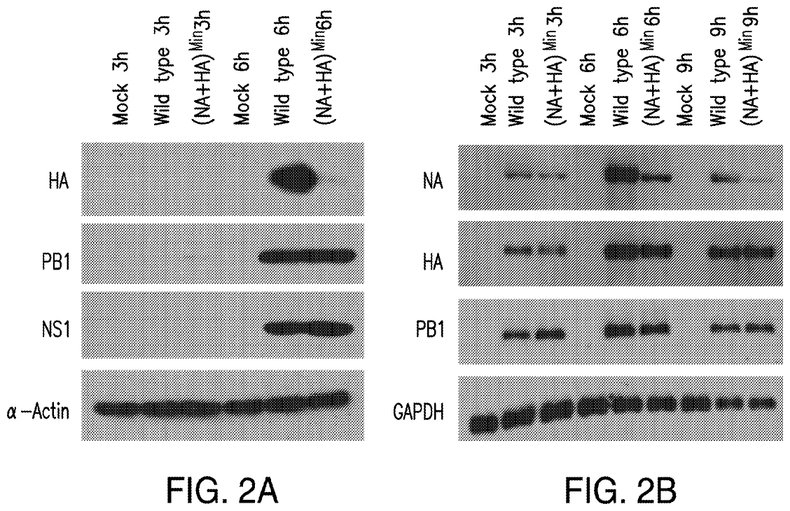

The apparent yield of HA polypeptide was examined by western blotting in MDCK cells at 3 h and 6 h post infection (p.i.) with 5 MOI of wt virus or (NA+HA)^{Min} Remarkably, at 6 h p.i., expression of HA protein was significantly reduced in (NA+HA)^{Min}-infected cells when compared to PR8-infected cells whereas PB1 and NS1 were synthesized to equal levels by viruses (FIG. 2A). Using the levels of PB1 and GAPDH mRNAs as control, the Northern blot analysis of mRNA levels in (NA+HA)^{Min}-infected cells indicated only a slight reduction of HA^{Min} mRNA at 3h and 6h (FIG. 2B).

In contrast, Northern blot analyses indicated an extensive reduction of the recoded NA^{Min} mRNA after 6h and particularly after 9 h p.i. (FIG. 2B). Early in infection (3h), the level of NA^{Min} mRNA was slightly reduced.

### Example 3—Characterization of the Reduced Codon-Pair Bias Variants as Vaccine Candidates in Mice

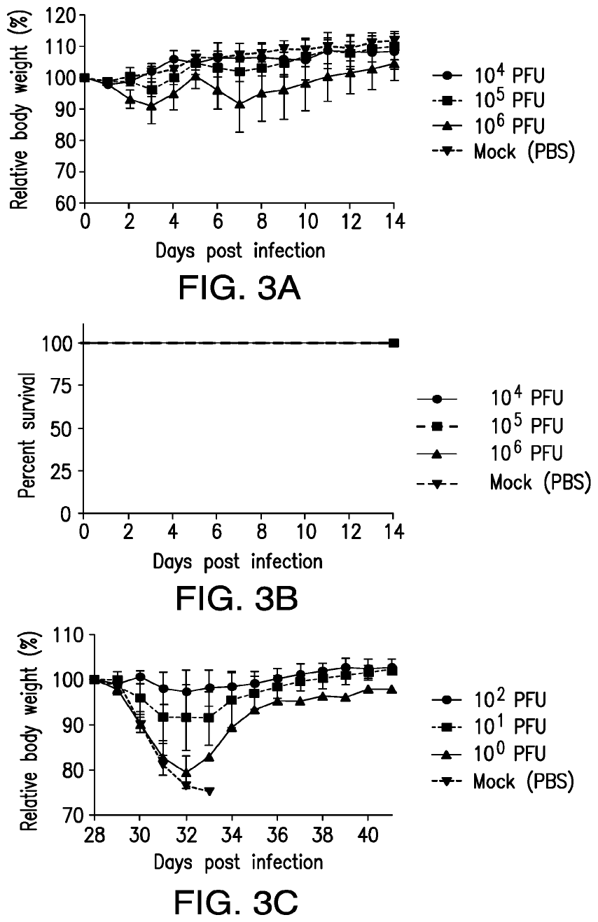

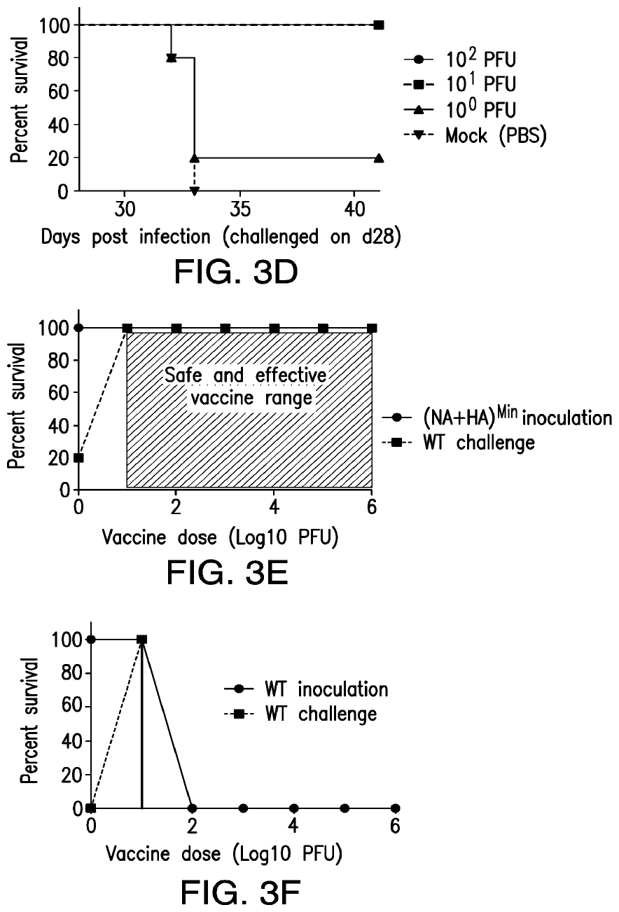

The growth phenotype and pathogenesis of the (NA+HA)^{Min} variant was examined in an animal model. Groups of five BALB/c mice received (NA+HA)^{Min} at doses of 10⁴, 10⁵ or 10⁶ PFU intra-nasally, and body weight and survival of the animals was monitored continuously for 14 days p.i. (FIG. 3A, B). Morbidity and mortality (weight loss, reduced activity, death) was monitored. The Lethal Dose 50 (LD₅₀) of the wildtype virus and the vaccine candidates was calculated by the method of Reed and Muench (Reed, L. J.; Muench, H., 1938, The American Journal of Hygiene 27: 493-497). Remarkably, the (NA+HA)^{Min} variant did not induce apparent disease after a dose up to 10⁵ PFU. Even at 10⁶ PFU, mice only suffered transient weight loss, but all animals survived. Therefore, the theoretical LD₅₀ of the (NA+HA)Min variant was calculated to be equal or greater than 3.16×10⁶ PFU, which exceeds that of wt PR8 by a factor of at least 100,000 (Table 1).

Whereas the (NA+HA)^{Min}, HA^{Min}, and NA^{Min} variants replicated with nearly equal efficiency and similar kinetics as wt PR8 in MDCK cells (FIG. 1C), the LD₅₀ of the variants were by orders of magnitude different: PR8=32 PFU, HA^{Min} =1.7×10³PFU (13), NA^{Min}=2.4×10⁵ PFU (FIG. 7, Table 5), and (NA+HA)^{Min} >3.3×10⁶. By itself, the NA^{Min} gene is about 100-fold more attenuated than the HA^{Min} gene, but reducing expression of NA and NP in the same virus significantly increases attenuation of the virus.

TABLE 5
LD₅₀ and PD₅₀ of Attenuated Virus

| | LD₅₀ | PD₅₀ |
|---|---|---|
| WT PR8 | 3.2 × 10¹ | ~1 |
| NA^{Min} | 2.4 × 10⁵ | <32 |
| HA^{Min} | 1.7 × 10³ | n.d. |
| (NA + HA)^{Min} | >3.3 × 10⁶ | 2.4 |

Vaccine candidates should be capable of providing, at low dose, long-term protection from challenge with a lethal dose of wt virus. The dose of (NA+HA)^{Min} required to protect 50% of vaccinated animals from subsequent lethal wild type challenge (defined as “protective dose 50”, PD₅₀) was determined. Groups of five Balb/c mice were vaccinated with a single dose of 10⁰, 10¹, or 10² PFU of (NA+HA)^{Min}. 28 days after vaccination, the animals were challenged with 10⁵ PFU (3000×LD₅₀) of wt PR8 virus. As with the original infections, we monitored body weight and survival of the animals 14 days after challenge. Remarkably, although (NA+HA)^{Min} was highly attenuated in mice, it was also highly proficient at protecting against lethal challenge with wt virus. As little as 10 PFU of (NA+HA)^{Min} protected all five mice from lethal challenge (FIG. 3C, 3D). The PD₅₀ value calculated by the method of Reed-Muench was only 2.4 PFU. (Table 5) To our knowledge this is the lowest reported protective dose of an experimental vaccine in a mouse model.

Vaccine safety and protective range was evaluated with various doses of either (NA+HA)^{Min} variant or wt PR8. As shown in FIG. 3E, a zone of five orders of magnitude (from 10 PFU to 10⁶ PFU) can be considered the “region of safety” of (NA+HA)^{Min} vaccination since all mice receiving increasing doses of “vaccine” within this region were protected from lethal challenge with wt virus. In contrast, the safe and effective region for wt PR8 was extremely limited (FIG. 3F).

### Example 4—the Growth of (NA+HA)Min is Greatly Reduced in the Lungs of Vaccinated Mice

To determine parameters of the (NA+HA)^{Min} pathogenicity in vivo, groups of BALB/c mice were infected with 10⁴ PFU of wt PR8 or (NA+HA)^{Min}. On day 1, 3, 5, 7, 9 and 11, three mice each from the wt and (NA+HA)^{Min} groups were euthanized, their lungs were homogenized, and virus titers in the homogenates were determined by plaque assays. As expected, wt PR8 replicated well, but even (NA+HA)^{Min} replicated noticeably in lungs of the vaccinated animals. Both PR8 and variant achieved maximum titers around day 3 (FIG. 4A) although there was a ˜100 fold difference in the titers between the two viruses. All wt PR8-infected mice died on day 5, whereas all (NA+HA)^{Min}-infected mice remained healthy. (NA+HA)^{Min} was eventually cleared at 8 to 9 days p.i. (FIG. 4A). When mice were inoculated at different doses, the (NA+HA)^{Min} titers were always 100-1000 fold lower in lungs when compare to those of wt PR8 on day 3 p.i. (FIG. 4B). Strikingly, at a vaccination dose of 10 PFU when (NA+HA)^{Min} barely replicated in the lungs of the animals, it nevertheless provided complete protection against wt PR8 challenge (FIGS. 4B and 3D). Interestingly, the attenuation of (NA+HA)Min in mice correlates with the attenuation of (NA+HA)^{Min} in A549 cells (FIG. 1D).

### Example 5—Cross Protection and Long Term Protection Induced by the (NA+HA)^{Min} Variant

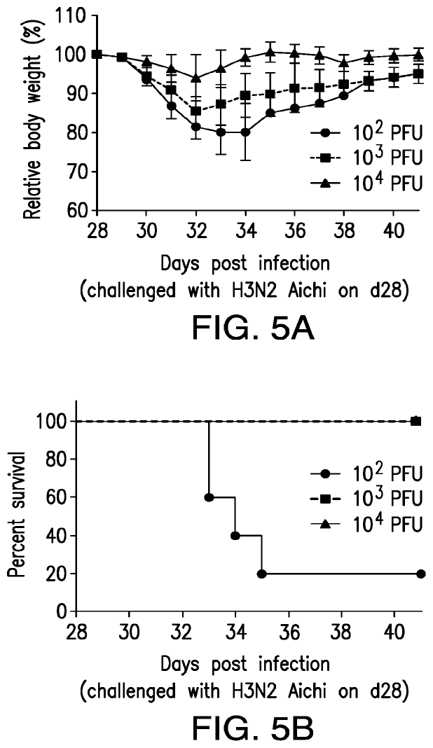

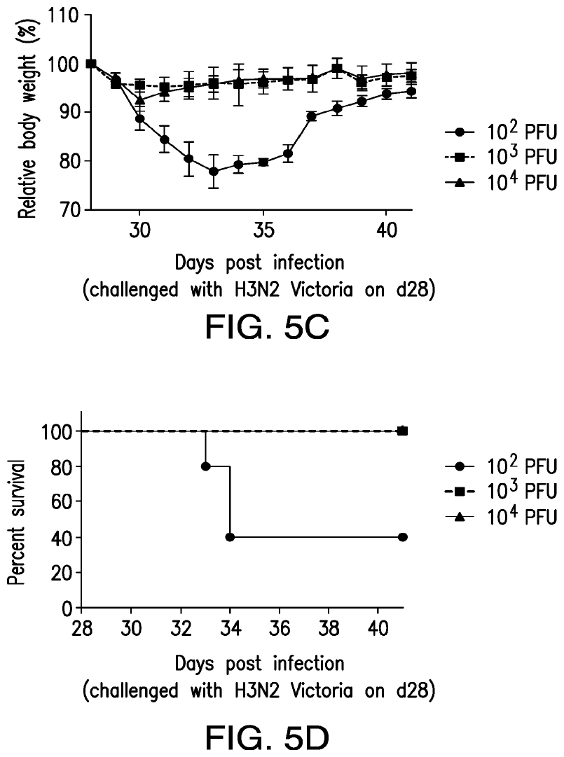

The (NA+HA)^{Min} variant of PR8, which belongs to the influenza H1N1 subtype, was further tested for its capacity to cross protect animals against infections with a heterologous influenza virus strain, such as a mouse adapted H3N2 strain (A/Aichi/2/1968) (21). Groups of five BALB/c mice were vaccinated with (NA+HA)^{Min} virus at doses ranging from 10² to 10⁴ PFU and challenged 28 days post vaccination with 100×LD₅₀ doses of A/Aichi/2/1968 (H3N2) virus (1.5×10⁴ PFU). Remarkably, 1000 PFU of (NA+HA)^{Min} were sufficient to protect mice from the heterologous lethal challenge, corresponding to a PD₅₀ value of only 237 PFU (FIG. 5A, 5B). A similar result was obtained when the vaccinated (NA+HA)^{Min} mice were challenged with a different strain of mouse adapted H3N2, A/Victoria/3/75. Again, as little as 1000 PFU of the H1N1 PR8-(NA+HA)^{Min} variant protected all mice from lethal challenge with 100×LD₅₀ dose (3.2×10⁴ PFU) of A/Victoria/3/75. The PD₅₀ of (NA+HA)^{Min} protecting against A/Victoria/3/75 (H3N2) was only 147 PFU (FIG. 5C, 5D). Both results indicate that (NA+HA)^{Min} of H1N1 PR8 can induce a robust cross protective immune response in mice against H3N2 subtypes.

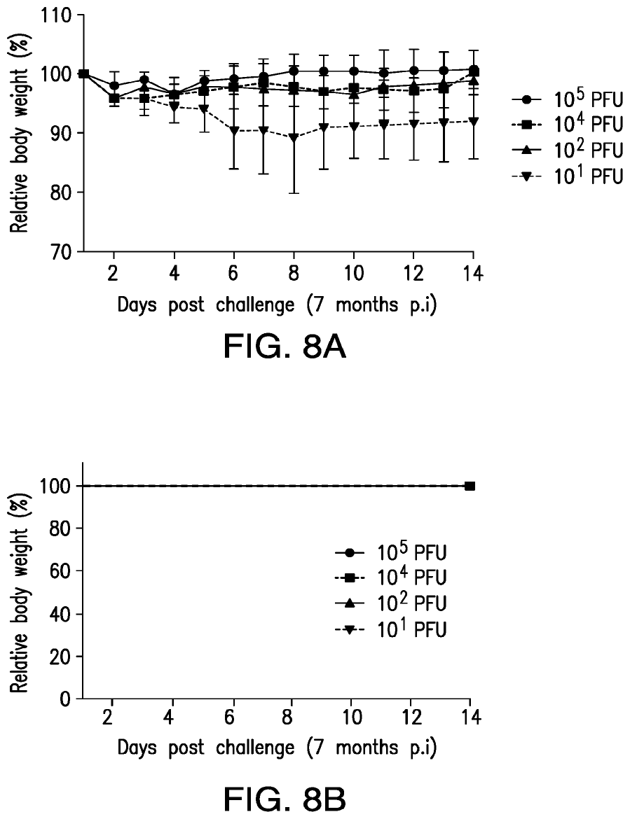

(NA+HA)^{Min}-vaccinated animals were tested to determine whether they were protected against challenge after an extended period of time. Groups of five mice were vaccinated with different doses (10¹ to 10⁵ PFU) of (NA+HA)^{Min} and the animals were challenged seven months later with 10⁵ PFU of wt PR8. All vaccinated animals were completely protected without signs of disease (FIG. 8).

### Example 6—the (NA+HA)^{Min} Variant Induces a Robust Antibody Response

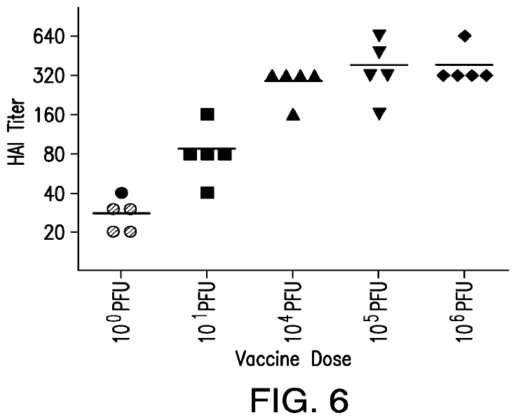

The host response to (NA+HA)^{Min} inoculation suggested a strong host response, including adaptive immunity. Groups of five Balb/c mice were vaccinated with varying doses of (NA+HA)^{Min} or wt PR8 (see FIG. 6). Sera were collected on day 28 p.i., and antibody responses were determined by hemagglutination inhibition (HAI) assays performed according to the protocol in the WHO Manual on Animal Influenza Diagnosis and Surveillance (23). The mice were challenged with a lethal dose of PR8 (10⁵ PFU). An HAI titer of 40 or more in the serum is generally considered to be protective (22). This level was reached with just 10¹ PFU of (NA+HA)^{Min} (FIG. 6) and protected vaccinated mice from challenge with 10⁵ PFU wt PR8 virus (FIG. 6).

### Example 7—Virus Composition

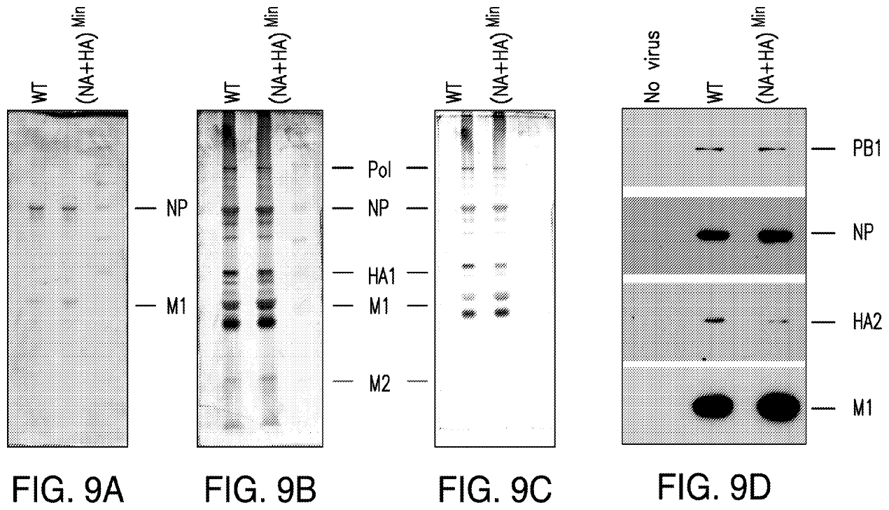

Both WT and (NA+HA)^{Min} virus were purified by sucrose gradient. 5×10⁷ PFU of both viruses were loaded onto SDS gels followed by Commassie blue stain (0.1% Coomassie blue R250 for 45 min.) (FIG. 9A) or silver stain (Bio-Rad silver stain kit) (FIGS. 9B and C) to detect virion protein composition. At the same PFU, WT virions contain more HA1 molecules than the (NA+HA)^{Min} virus, while the latter contains more M1 proteins.

WT and (NA+HA)^{Min} virus were also analyzed by Western blot. 2×10⁷ PFU of WT and (NA+HA)^{Min} viruses were loaded onto SDS-PAGE gels and analyzed for content of PB1, NP, HA2, and M1. At the same PFU of purified virions, the virus preparations have similar amounts of NP and PB1 protein. Purified WT virions, however, have more HA2 protein, while purified (NA+HA)^{Min} virions have more M1 protein. (FIG. 9D).

### Example 8—Expression of Virus Proteins and mRNAs in Infected MDCK Cells

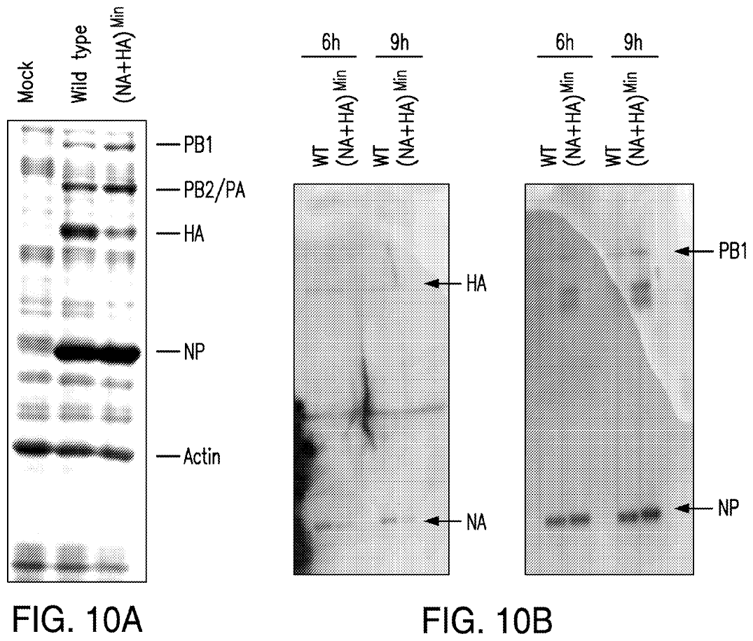

HA protein expression was measured by ³⁵S methionine incorporation. MDCK cells were infected with 10 MOI wild type PR8, or (NA+HA)^{Min} virus. At 3 h post infection, cells were starved for 45 min, and then labeled for 30 min. Following cell lysis, equal amounts of cell lysates were resolved by SDS PAGE and labeled proteins were visualized by autoradiography. Expression of the HA protein is notably reduced relative to other viral proteins in (NA+HA)^{Min} virus-infected cells. (FIG. 10A).

Viral mRNA in virus infected MDCK cells nucleus was analyzed by Northern blot. MDCK cells were infected with both WT and (NA+HA)^{Min} viruses at an MOI of 1. At 6h, and 9h post infection, cells were lysed using Life Technologies PARIS Kit. Nucleus and cytoplasmic portions were separated and mRNA were extracted from both portions. Northern blotting was performed using isolated mRNAs. The nuclear NP mRNA signals were relatively similar between WT and (NA+HA)^{Min} virus infected cells at all time points. Yet, WT virus infected cells, compared to (NA+HA)^{Min} viruses infected cells, contained more nuclear HA and NA mRNA, and less nuclear PB1 mRNA. (FIG. 10B)

### Example 9—Passive Immunization by Serum Transfer from PR8-(NA+HA)^{Min} Vaccinated Mice Protects Naïve Mice from Homologous WT PR8 Challenge

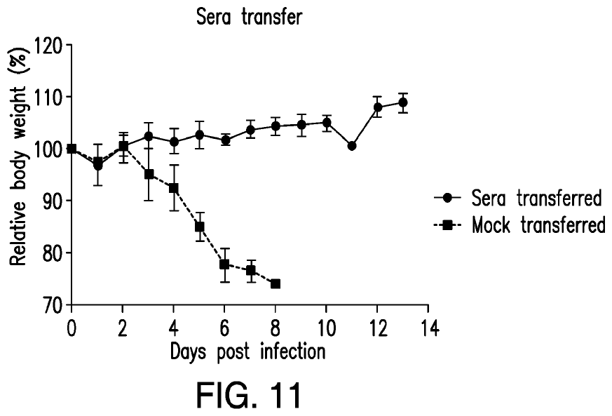

Groups of five Balb/C mice were vaccinated with 10⁴ PFU (NA+HA)^{Min} virus or PBS. 28 days after vaccination mouse sera were collected, and transferred to five naïve Balb/C mice in a volume of 250 ul. 24 h post transfer, mice were challenged with 10⁵ PFU of WT PR8, corresponding to 3000× LD₅₀. All passively immunized mice survived and remained healthy upon challenge, while mock transferred mice died in 8 days. These results suggest that antibodies are the major mediator of immune protection induced by (NA+HA)^{Min} virus vaccination. (FIG. 11).

### Example 10—Passive Immunization by Serum Transfer from PR8-(NA+HA)^{Min} Vaccinated Mice Protects Naïve Mice from Heterologous H3N2 Challenge

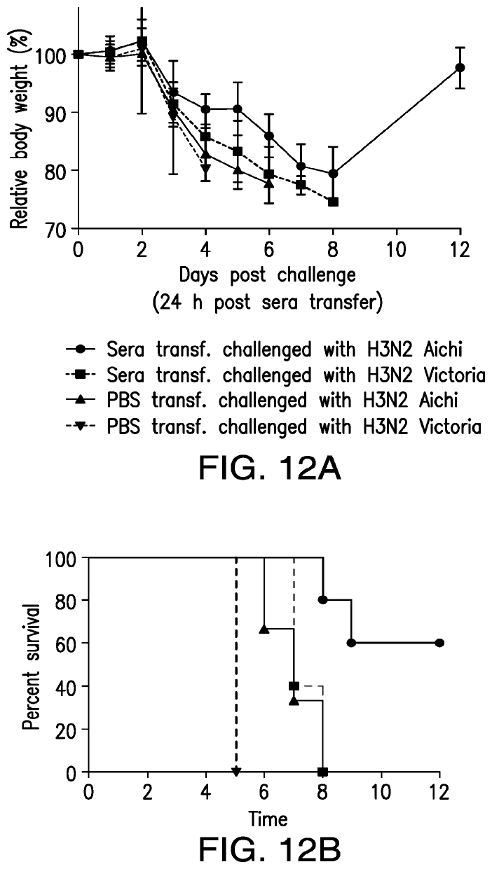

Groups of five Balb/C mice were infected with 3×10⁵ PFU H1N1-(NA+HA)^{Min} virus or PBS. On day 28, all mice were euthanized and their blood was collected. Sera were prepared on the same day and immediately transferred to groups of five naïve Balb/c mice (i.p injection with 250 μl of sera). 24 h post transfer, mice were challenged with 10×LD₅₀ of H1N1—WT PR8, H3N2 Aichi or H3N2 Victoria viruses. Their body weights (FIG. 12A) and survival rates (FIG. 12B) were monitored for 14 days post infection. 60% of sera transferred mice were protected from lethal H3N2 Aichi challenge, and survival times upon challenge of lethal H3N2 Victoria virus were extended.

### Example 11—Cross Protection

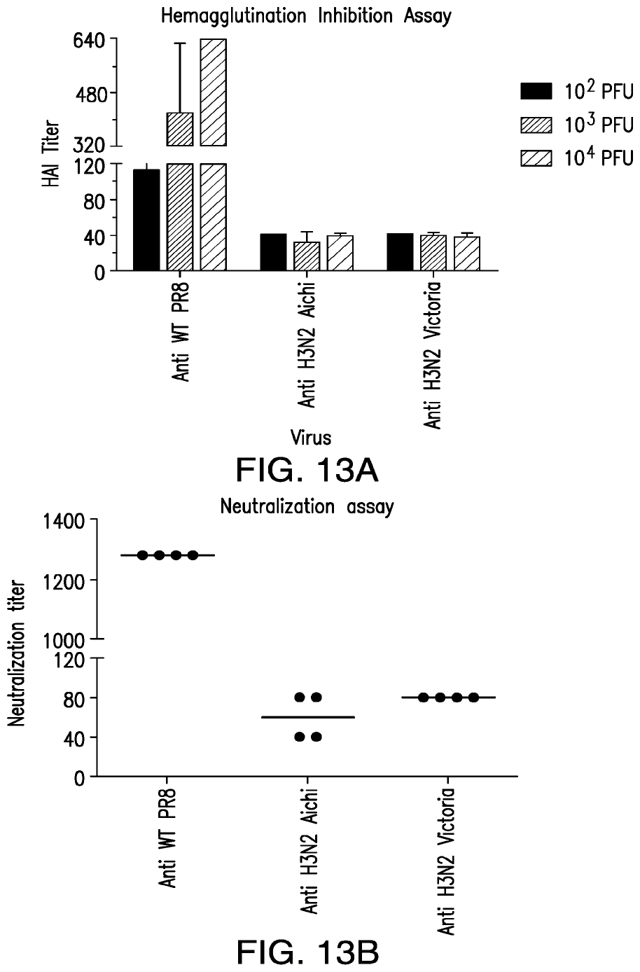

Cross protection was investigated by assay of hemagglutination inhibition and neutralization. To determine inhibition of hemagglutination, groups of five Balb/C mice were vaccinated with 10²—10⁴ PFU of H1N1-(NA+HA)^{Min} virus. Sera were collected on day 28 p.i. Hemagglutination inhibition assays were performed by incubating the serum with H1N1 PR8, H3N2 Aichi or H3N2 Victoria virues. (FIG. 13A). H1N1-(NA+HA)^{Min} virus infected mice contain abundant anti-H1N1 HA antibodies with a HAI titer from 100-640. The sera, however, do not contain much of the anti-H3N2 HA antibodies, since the HAI titer are 40 regardless of the vaccine dose. This data suggests that survival of (NA+HA)^{Min} virus-vaccinated mice from heterologous challenge (as illustrated in Example 5) is mainly due to immunity not correlated with antibodies, such as cellular immunity.

To test neutralization, MDCK cells were seeded onto 96 well plate on day 0. 2 fold dilutions of sera from vaccinated mice were incubated with 100 TCID₅₀ viruses for 1h and then added to pre-seeded MDCK cells on day 1. Cells were stained with crystal violet on day 4 to determine neutralization titers.

Groups of five Balb/C mice were vaccinated with 10⁵ PFU of H1N1-(NA+HA)^{Min} virus. Sera were collected on day 28 p.i. Neutralization assays were performed by incubating the sera with H1N1 PR8, H3N2 Aichi or H3N2 Victoria viruses. H1N1-(NA+HA)^{Min} virus infected mice were capable of neutralizing H1N1 PR8 with a neutralization titers above 1200. The sera, interestingly, were also able to neutralize H3N2 viruses. (FIG. 13B).

### Example 12—Neuraminidase Encoded by (NA+HA)^{Min}

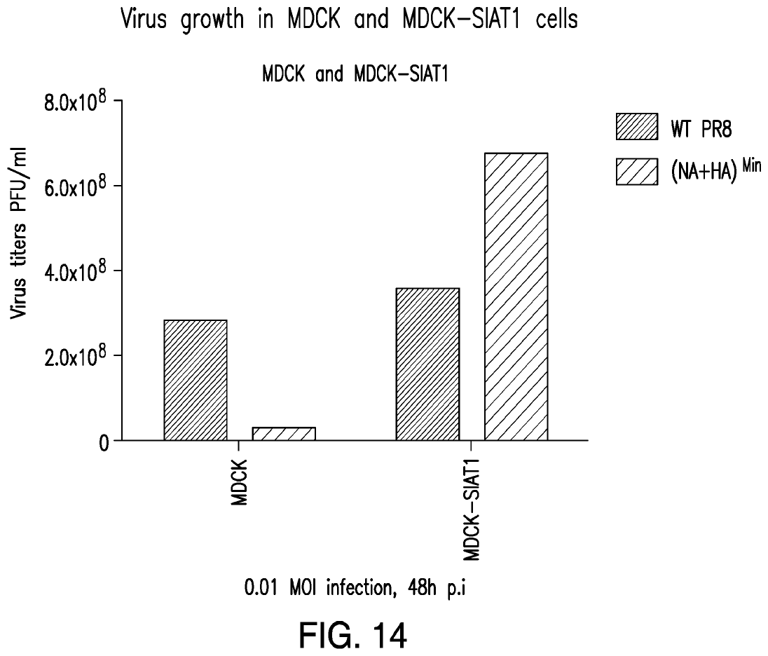

Viral neuraminidase cleaves terminal sialic acid residues from glycan structures on the surface of an infected cell, which promotes the release of progeny viruses. MDCK cells and MDCK-SIAT1 cells which overexpress overexpressing the α-2,6-Sialyltransferase, were infected with WT or (NA+HA)^{Min} viruses at MOI of 0.01. Virus titers were examined at 48 h p.i. In MDCK-SIAT1 cells, which overexpressed influenza receptor sialic acid, both WT and (NA+HA)^{Min} viruses grew better than MDCK cell lines. (FIG. 14). This indicates that (NA+HA)^{Min} virus comprises neuraminidase molecules encoded by NA^{Min} that cleave sialic acid residues normally.

### Example 13—T and B Cell Responses in Lungs and Spleen

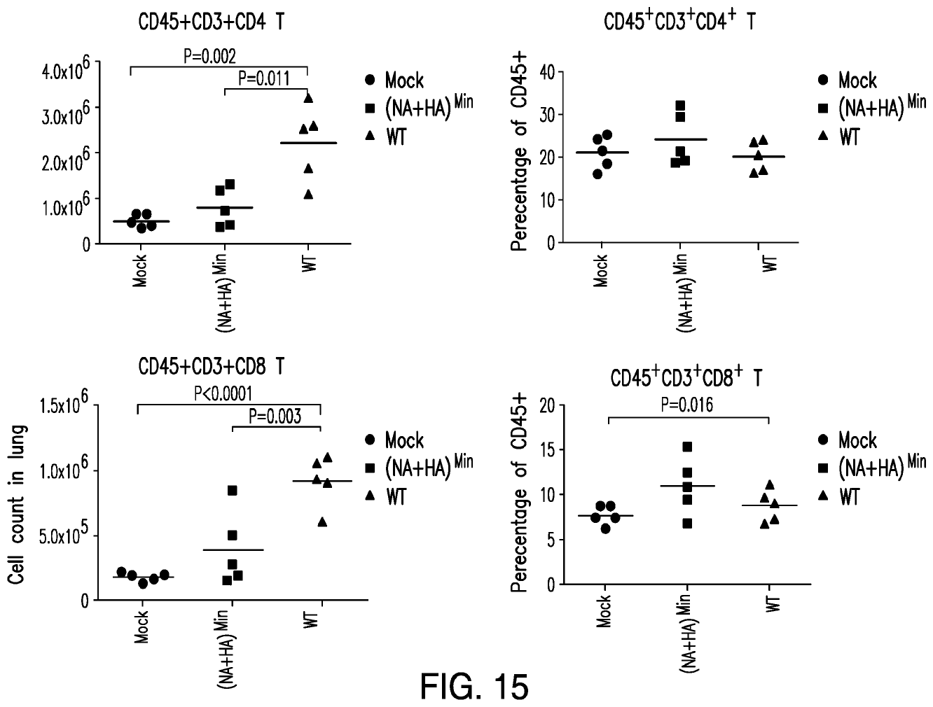

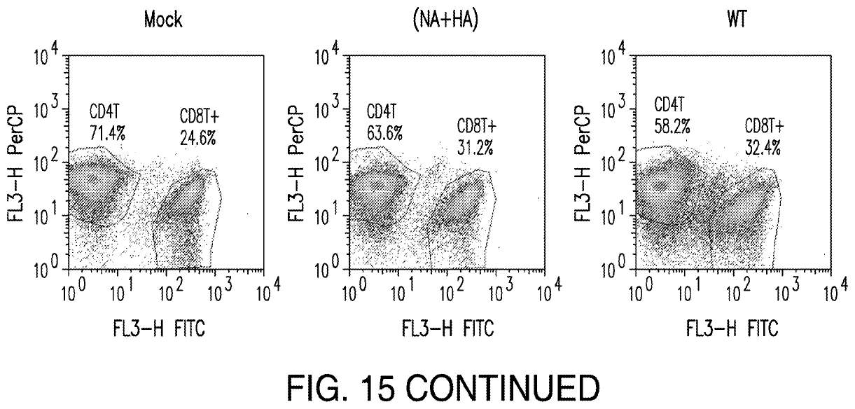

T cell responses in lungs. Groups of five Balb/C mice were with 10 PFU of WT (a dose close the LD₅₀ of this virus) or 10 PFU (NA+HA)^{Min} (a dose over 300,000-fold below the LD₅₀ for this virus). On day 7 post infection, mice were euthanized and their lungs were collected for flow cytometry. (NA+HA)^{Min} infected mice showed lower numbers of CD4⁺ T and CD8⁺ T cells than WT-infected mice, since (NA+HA)^{Min} infection is cleared by 7 days, while WT infection is still ongoing. (FIG. 15).

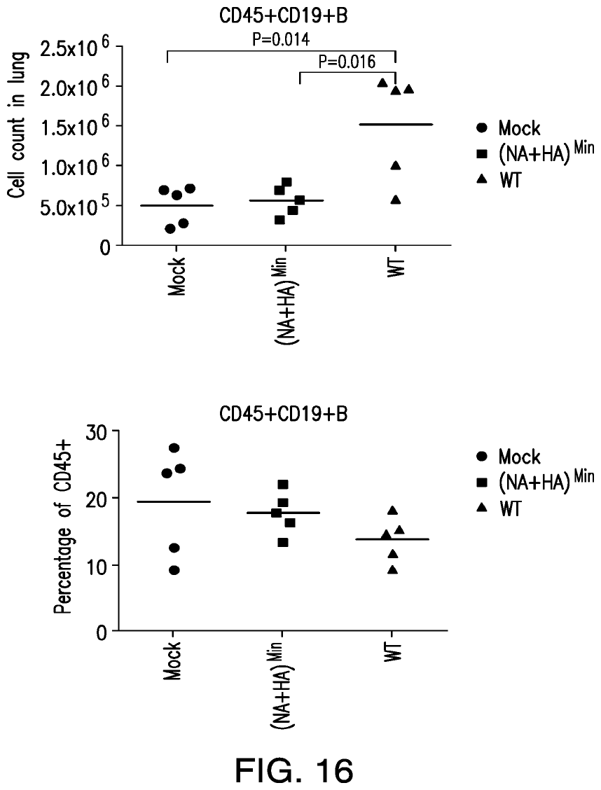

B cell responses in lungs. Groups of five Balb/C mice were infected with 10 PFU of WT or (NA+HA)^{Min} viruses. On day 7 post infection, mice were euthanized and their lungs were collected for flow cytometry. WT infected mice showed higher numbers of B cells than both the (NA+HA)^{Min} viruses and the mock group, indicating the WT viruses were much harder to clear than the other two. Yet, the percentage of CD45⁺ B cells in (NA+HA)^{Min} virus infected mice were similar, or slightly higher, than the WT PR8 infected mice, which indicates they share similar ability in inducing long term protective antibodies. (FIG. 16)

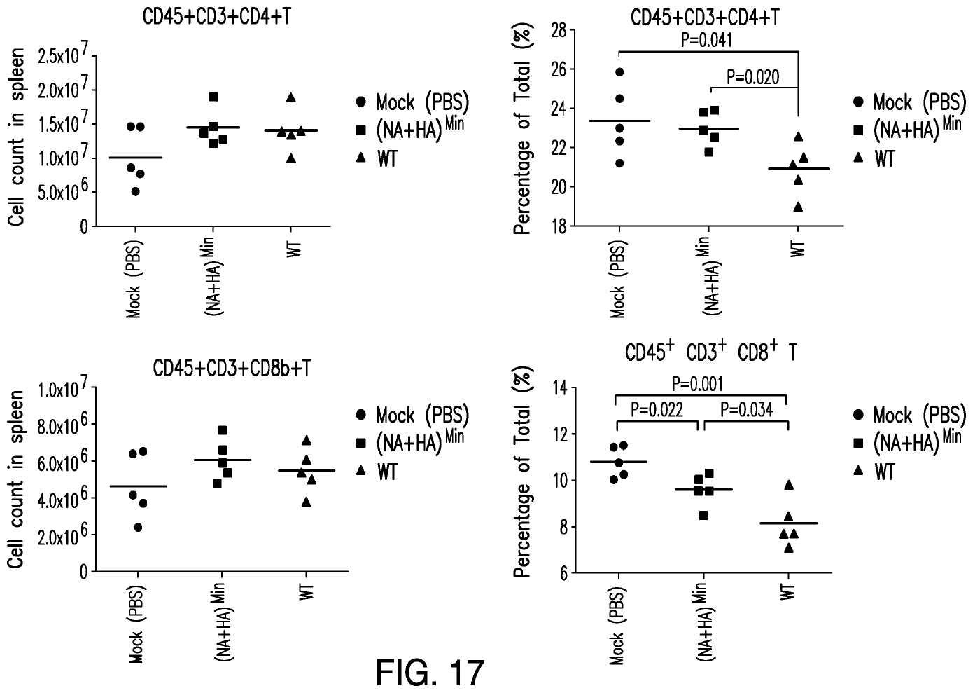

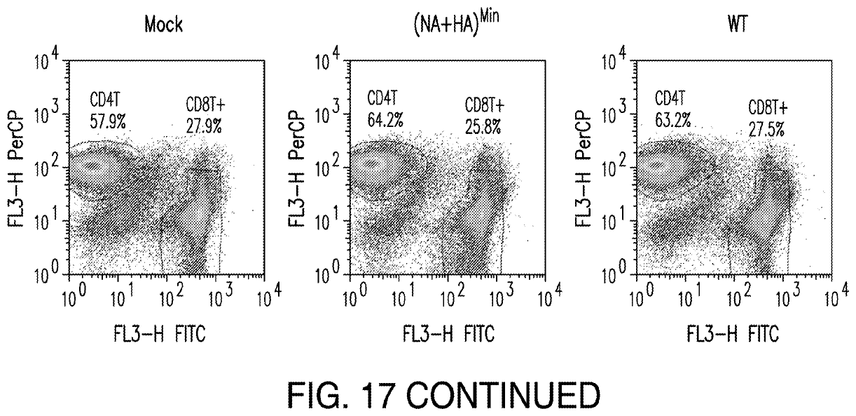

T cell responses in spleen. Groups of five Balb/C mice were infected with 10 PFU of WT or (NA+HA)^{Min} viruses. On day 7 post infection, mice were euthanized and their spleens were collected for flow cytometry. Both WT and (NA+HA)^{Min} virus infected mice showed higher number of CD4⁺ T and CD8⁺ T cells than the mock group, indicating a strong adaptive immune responses triggered by both viruses. (FIG. 17).

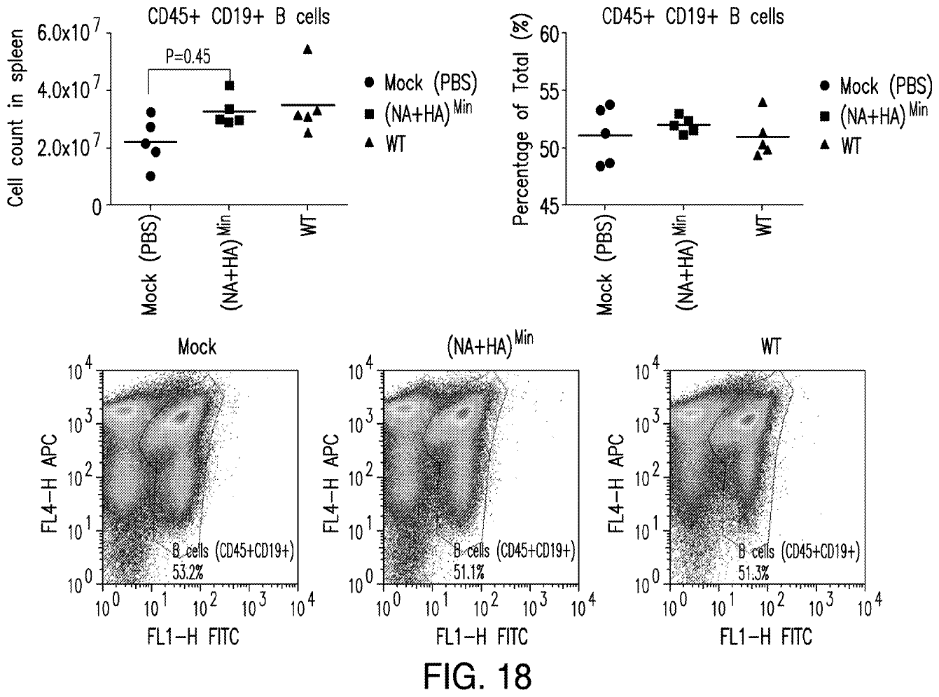

B cell responses in spleen. Groups of five Balb/C mice were infected with 10 PFU of WT or (NA+HA)^{Min} viruses. On day 7 post infection, mice were euthanized and their spleens were collected for flow cytometry. (NA+HA)^{Min} infected mice showed significantly higher numbers of B cells than mock group, indicating (NA+HA)^{Min} virus is highly efficient in inducing protective antibodies. (FIG. 18).

In summary, at 7 days post infection, the response to WT virus in lung tissue involved greater numbers of CD4⁺ T, CD8⁺ T and B cells to clear the viruses than the response to (NA+HA)^{Min}. In spleen, (NA+HA)^{Min} and WT infected mice both showed elevated T and B cells, indicating strong adaptive immune responses. Also, the T cell proportion of cells in spleen responding to infection by (NA+HA)^{Min} was higher than the proportion responding to infection by WT virus. (FIG. 17).

### Example 14—Flow Cytometry Analyses of Immune Cells Infiltrating Lung Tissue

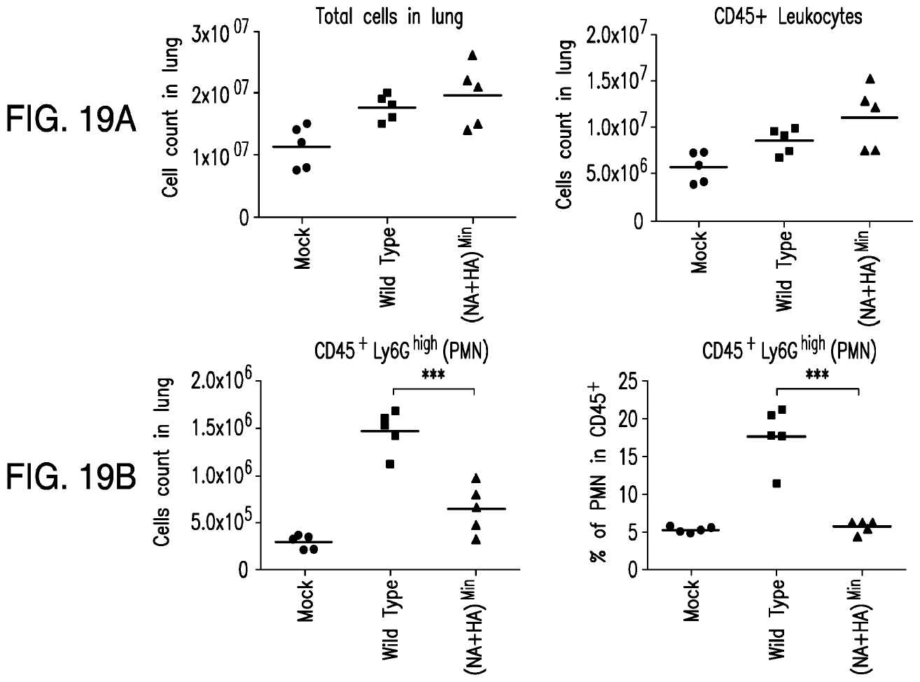

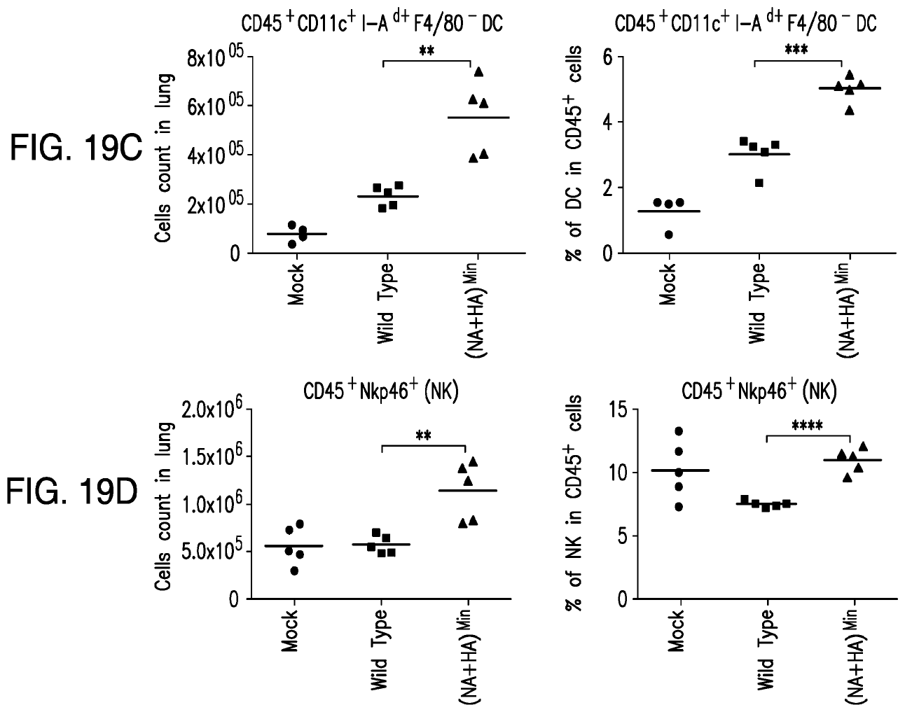

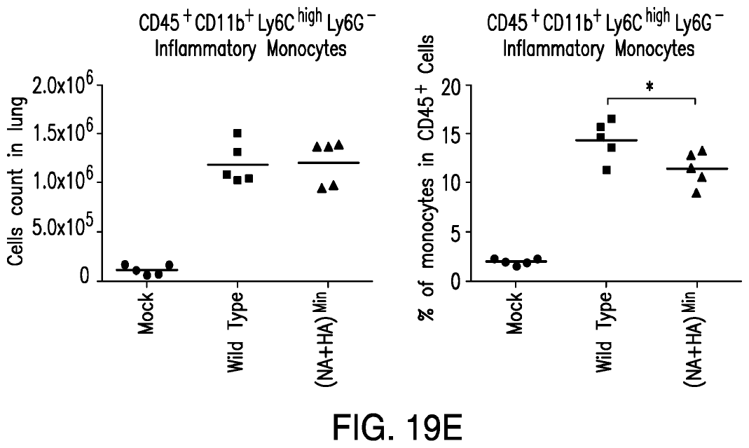

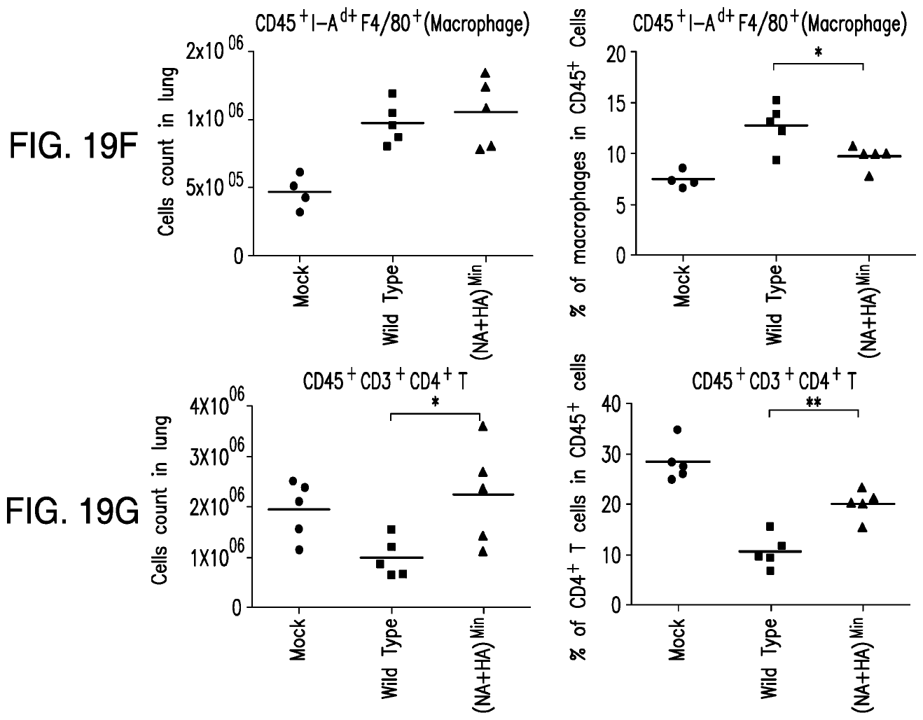

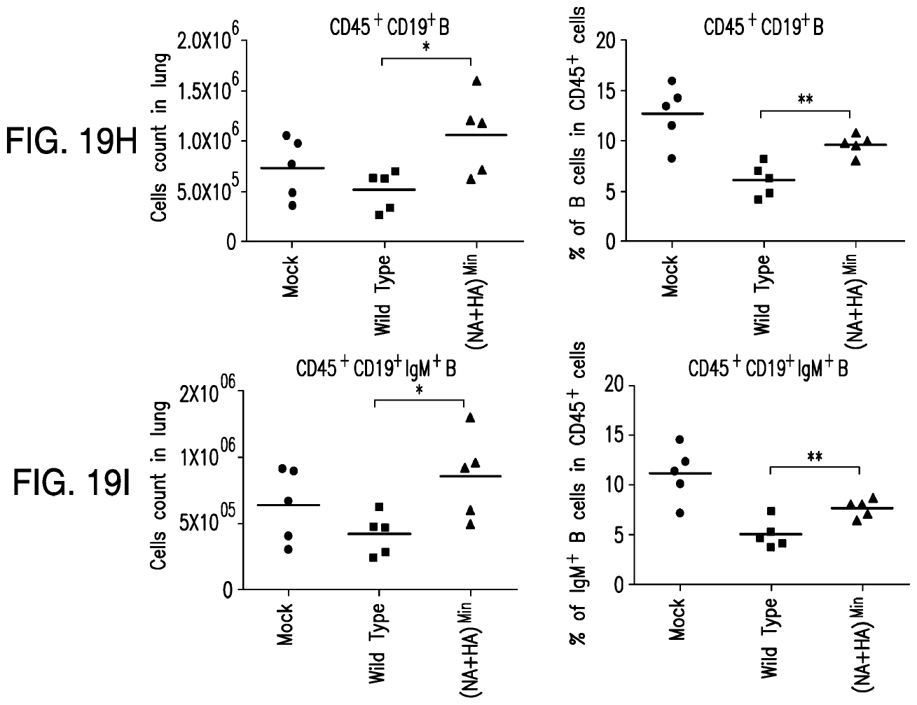

Groups of five male Balb/C mice received 10⁴ PFU wild type PR8 (a lethal dose equal to 300-fold the LD₅₀ for this virus), 10⁴ PFU (NA+HA)^{Min} (a safe dose at least 300-fold below the LD₅₀ for this virus), or PBS. Note: A the chosen dose of 10⁴ PFU wild type PR8-infected mice invariably succumb to the infection between 4 and 9 days. On day 3 post infection, lungs were collected and flow cytometry analyses were performed. FIG. 19 shows the results for various immune cells as follows: (A) CD45⁺ leukocytes, (B) CD45⁺ Ly6G^{high} polymorphonuclear leukocytes (PMN), (C) CD45⁺ CD11c⁺ I-A^{d+} F4/80⁻ dendritic cells, (D) CD45⁺ NKp46⁺ natural killer cells, (E) CD45⁺ CD11b⁺ Ly6C^{high}Ly6G⁻ inflammatory monocytes, (F) CD45⁺ I-A^{d+} F4/80⁺ macrophages, (G) CD45⁺ CD3⁺ CD4⁺ T helper cells, (H) CD45⁺ CD19⁺ B cells, and (I) CD45⁺ CD19⁺ IgM⁺ B cells were monitored. Most notably (NA+HA)^{Min} infection induced a significantly higher amount of natural killer cells, implicated in viral clearance, as well as a reduced infiltration of PMN, which are known to be associated with immune induced lung damage following natural influenza virus infection. Thus the marked lack of PMN infiltration during (NA+HA)^{Min} infection may explain the high degree of attenuation (i.e the absence of virus induced disease and pathology) of (NA+HA)^{Min}

### REFERENCES

1. Thompson, W. W., Comanor, L., & Shay, D. K. (2006) Epidemiology of seasonal influenza: use of surveillance data and statistical models to estimate the burden of disease. J. Infect. Dis. 194 Suppl 2:S82-91.
2. Smith, D. J., et al. (2004) Mapping the antigenic and genetic evolution of influenza virus. Science 305(5682):371-376.
3. Bouvier, N. M. & Palese, P. (2008) The biology of influenza viruses. Vaccine 26 Suppl. 4:D49-53.
4. Simonsen, L., et al. (2005) Impact of influenza vaccination on seasonal mortality in the U.S. elderly population. Arch. Intern. Med. 165(3):265-272.
5. Osterholm, M. T., Kelley, N. S., Sommer, A., & Belongia, E. A. (2012) Efficacy and effectiveness of influenza vaccines: a systematic review and meta-analysis. Lancet Infect. Dis. 12(1):36-44.
6. Belshe, R. B., et al. (2007) Live attenuated versus inactivated influenza vaccine in infants and young children. N. Engl. J Med. 356(7):685-696.
7. Hussain, A. I., Cordeiro, M., Sevilla, E., & Liu, J. (2010) Comparison of egg and high yielding MDCK cell-derived live attenuated influenza virus for commercial production of trivalent influenza vaccine: in vitro cell susceptibility and influenza virus replication kinetics in permissive and semi-permissive cells. Vaccine 28(22):3848-3855
8. Wang, Z., Tobler, S., Roayaei, J., & Eick, A. (2009) Live attenuated or inactivated influenza vaccines and medical encounters for respiratory illnesses among US military personnel. JAMA 301(9):945-953.
9. Gutman, G. A. & Hatfield, G. W. (1989) Nonrandom utilization of codon pairs in Escherichia coli. Proc. Natl. Acad. Sci U.S.A 86(10):3699-3703.
10. Moura, G., et al. (2007) Large scale comparative codon-pair context analysis unveils general rules that fine-tune evolution of mRNA primary structure. PLoS One 2(9):e847.
11. Wang, F. P. & Li, H. (2009) Codon-pair usage and genome evolution. Gene 433(1-2):8-15.
12. Coleman, J. R., et al. (2008) Virus attenuation by genome-scale changes in codon pair bias. Science 320(5884):1784-1787.
13. Mueller, S., et al. (2010) Live attenuated influenza virus vaccines by computer-aided rational design. Nat Biotechnol 28(7):723-726.
14. Cello, J., Paul, A. V., & Wimmer, E. (2002) Chemical synthesis of poliovirus cDNA: generation of infectious virus in the absence of natural template. Science 297(5583):1016-1018.
15. Sutejo, R., et al. (2012) Activation of type I and III interferon signalling pathways occurs in lung epithelial cells infected with low pathogenic avian influenza viruses. PLoS One 7(3):e33732.
16. Dove, B. K., et al. (2012) A quantitative proteomic analysis of lung epithelial (A549) cells infected with 2009 pandemic influenza A virus using stable isotope labelling with amino acids in cell culture. Proteomics 12(9):1431-1436.
17. Doma, M. K. & Parker, R. (2006) Endonucleolytic cleavage of eukaryotic mRNAs with stalls in translation elongation. Nature 440(7083):561-564.
18. Liu, C., Eichelberger, M. C., Compans, R. W., & Air, G. M. (1995) Influenza type A virus neuraminidase does not play a role in viral entry, replication, assembly, or budding. J Virol. 69(2):1099-1106.
19. Palese, P., Tobita, K., Ueda, M., & Compans, R. W. (1974) Characterization of temperature sensitive influenza virus mutants defective in neuraminidase. Virology 61(2):397-410.
20. Muster, T., et al. (1995) Mucosal model of immunization against human immunodeficiency virus type 1 with a chimeric influenza virus. J. Virol. 69(11):6678-6686.
21. Koutsonanos, D. G., et al. (2009) Transdermal influenza immunization with vaccine-coated microneedle arrays. PLoS One 4(3):e4773.
22. de Jong, J. C., et al. (2003) Haemagglutination-inhibiting antibody to influenza virus. Dev Biol (Basel) 115:63-73.
23. WHO (2002) WHO Manual on Animal Influenza Diagnosis and Surveillance. www.who.int vaccine research diseases influenza WHO manual on animaldiagnosis and surveillance 2002 5.pdf
24. Reed, L., Muench, M. (1938) A simple method for estimating fifty percent endpoints. Am J. Hyg 27(3):493-497.

1-11. (canceled)

12. An influenza virus genome having a hemagglutinin (HA) protein-encoding sequence and a neuraminidase (NA) protein-encoding sequence, wherein the HA protein-encoding sequence, the NA protein-encoding sequence, or both have a codon pair bias less than −0.1; and the codon pair bias is calculated relative to an influenza host.

13. The influenza virus genome of claim 12, wherein the HA protein-encoding sequence, the NA protein-encoding sequence, or both have a codon pair bias less than −0.2.

14. The influenza virus genome of claim 12, wherein the HA protein-encoding sequence, the NA protein-encoding sequence, or both have a codon pair bias less than −0.3.

15. The influenza virus genome of claim 12, wherein the HA protein-encoding sequence, the NA protein-encoding sequence, or both have a codon pair bias less than −0.4.

16. The influenza virus genome of claim 12, wherein the influenza host is a human, bird, or pig host.

17. The influenza virus genome of claim 16, wherein the influenza host is a human host.

18. An influenza virus, comprising the influenza virus genome of claim 12.

19. An vaccine composition for inducing a protective immune response in a subject, the vaccine composition comprising the influenza virus of claim 18.

20. A method of eliciting a protective immune response in a subject, the method comprising administering to the subject a prophylactically or therapeutically effective dose of a vaccine composition comprising influenza virus of claim 18.

21. The method of claim 20, the method further comprising administering to the subject at least one adjuvant.

22. The method of claim 20, wherein the immune response is cross-protective against a heterologous influenza virus.

23. A method of making an influenza virus genome having a HA protein-encoding sequence and a NA protein-encoding sequence, the method comprising:
(a) obtaining the nucleotide sequence encoding the HA protein of an influenza virus and the nucleotide sequence encoding the NA protein of an influenza virus;
(b) recoding the HA protein-encoding sequence, the NA protein-encoding sequence, or both, so that the HA protein-encoding sequence, the NA protein-encoding sequence, or both have a codon pair bias less than −0.1, wherein the codon pair bias is calculated relative to an influenza host.

24. The method of claim 23, wherein the HA protein-encoding sequence, the NA protein-encoding sequence, or both have a codon pair bias less than −0.2.

25. The method of claim 23, wherein the HA protein-encoding sequence, the NA protein-encoding sequence, or both have a codon pair bias less than −0.3.

26. The method of claim 23, wherein the HA protein-encoding sequence, the NA protein-encoding sequence, or both have a codon pair bias less than −0.4.

27. The method of claim 23, wherein the influenza host is a human, bird, or pig host.

28. The method of claim 27, wherein the influenza host is a human host.
